SEQUENCE LISTING (53 sequences)

SEQ ID NO: 1 - protein, 476 aa, Homo sapiens [sequence omitted]

SEQ ID NO: 2 - protein, 473 aa, Mus musculus
MAKLLLVTGLALLLNAQLGSAYNLICYFTNWAQYRPGLGSFKPDDINPCLCTHLIYAFAGMQNNEITTIEWNDVTLYKAFNDLKNRNSKLKTLLAIGGWNFGTAPFTTMVSTSQNRQTFITSVIKFLRQYGFDGLDLDWEYPGSRGSPPQDKHLFTVLVKEMREAFEQEAIESNRPRLMVTAAVAGGISNIQAGYEIPELSKYLDFIHVMTYDLHGSWEGYTGENSPLYKYPTETGSNAYLNVDYVMNYWKNNGAPAEKLIVGFPEYGHTFILRNPSDNGIGAPTSGDGPAGPYTRQAGFWAYYEICTFLRSGATEVWDASQEVPYAYKANEWLGYDNIKSFSVKAQWLKQNNFGGAMIWAIDLDDFTGSFCDQGKFPLTSTLNKALGISTEGCTAPDVPSEPVTTPPGSGSGGGSSGGSSGGSGFCADKADGLYPVADDRNAFWQCINGITYQQHCQAGLVFDTSCNCCNWP

SEQ ID NO: 3 - protein, 466 aa, Homo sapiens
MVRSVAWAGFMVLLMIPWGSAAKLVCYFTNWAQYRQGEARFLPKDLDPSLCTHLIYAFAGMTNHQLSTTEWNDETLYQEFNGLKKMNPKLKTLLAIGGWNFGTQKFTDMVATANNRQTFVNSAIRFLRKYSFDGLDLDWEYPGSQGSPAVDKERFTTLVQDLANAFQQEAQTSGKERLLLSAAVPAGQTYVDAGYEVDKIAQNLDFVNLMAYDFHGSWEKVTGHNSPLYKRQEESGAAASLNVDAAVQQWLQKGTPASKLILGMPTYGRSFTLASSSDTRVGAPATGSGTPGPFTKEGGMLAYYEVCSWKGATKQRIQDQKVPYIFRDNQWVGFDDVESFKTKVSYLKQKGLGGAMVWALDLDDFAGFSCNQGRYPLIQTLRQELSLPYLPSGTPELEVPKPGQPSEPEHGPSPGQDTFCQGKADGLYPNPRERSSFYSCAAGRLFQQSCPTGLVFSNSCKCCTWN

SEQ ID NO: 4 - protein, 464 aa, Mus musculus
MVQSLAWAGVMTLLMVQWGSAAKLVCYLTNWSQYRTEAVRFFPRDVDPNLCTHVIFAFAGMDNHQLSTVEHNDELLYQELNSLKTKNPKLKTLLAVGGWTFGTQKFTDMVATASNRQTFVKSALSFLRTQGFDGLDLDWEFPGGRGSPTVDKERFTALIQDLAKAFQEEAQSSGKERLLLTAAVPSDRGLVDAGYEVDKIAQSLDFINLMAYDFHSSLEKTTGHNSPLYKRQGESGAAAEQNVDAAVTLWLQKGTPASKLILGMPTYGRSFTLASSSDNGVGAPATGPGAPGPYTKDKGVLAYYEACSWKERHRIEDQKVPYAFQDNQWVSFDDVESFKAKAAYLKQKGLGGAMVWVLDLDDFKGSFCNQGPYPLIRTLRQELNLPSETPRSPEQIIPEPRPSSMPEQGPSPGLDNFCQGKADGVYPNPGDESTYYNCGGGRLFQQSCPPGLVFRASCKCCTWS

SEQ ID NO: 5 - protein, 383 aa, Homo sapiens
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRFSKIASNTQSRRTFIKSVPPFLRTHGFDGLDLAWLYPGRRDKQHFTTLIKEMKAEFIKEAQPGKKQLLLSAALSAGKVTIDSSYDIAKISQHLDFISIMTYDFHGAWRGTTGHHSPLFRGQEDASPDRFSNTDYAVGYMLRLGAPASKLVMGIPTFGRSFTLASSETGVGAPISGPGIPGRFTKEAGTLAYYEICDFLRGATVHRTLGQQVPYATKGNQWVGYDDQESVKSKVQYLKDRQLAGAMVWALDLDDFQGSFCGQDLRFPLTNAIKDALAAT

SEQ ID NO: 6 - protein, 381 aa, Mus musculus
MGMRAALTGFAVLMLLQSCSAYKLVCYFTSWSQYREGVGSFLPDAIQPFLCTHIIYSFANISSDNMLSTWEWNDESNYDKLNKLKTRNTNLKTLLSVGGWKFGEKRFSEIASNTERRTAFVRSVAPFLRSYGFDGLDLAWLYPRLRDKQYFSTLIKELNAEFTKEVQPGREKLLLSAALSAGKVAIDTGYDIAQIAQHLDFINLMTYDFHGVWRQITGHHSPLFQGQKDTRFDRYSNVNYAVQYMIRLGAQASKLLMGIPTFGKSFTLASSENQLGAPISGEGLPGRFTKEAGTLAYYEICDFLKGAEVHRLSNEKVPFATKGNQWVGYEHKESVKNKVGFLKEKKLAGAMVWALDLDDFQGTCQPKEFFPLTNAIKDALA

SEQ ID NO: 7 - protein, 678 aa, Homo sapiens
MWKLLLWVGLVLVLKHHDGAAHKLVCYFTNWAHSRPGPASILPHDLDPFLCTHLIFAFASMNNNQIVAKDLQDEKILYPEFNKLKERNRELKTLLSIGGWNFGTSRFTTMLSTFANREKFIASVISLLRTHDFDGLDLFFLYPGLRGSPMHDRWTFLFLIEELLFAFRKEALLTMRPRLLLSAAVSGVPHIVQTSYDVRFLGRLLDFINVLSYDLHGSWERFTGHNSPLFSLPEDPKSSAYAMNYWRKLGAPSEKLIMGIPTYGRTFRLLKASKNGLQARAIGPASPGKYTKQEGFLAYFEICSFVWGAKKHWIDYQYVPYANKGKEWVGYDNAISFSYKAWFIRREHFGGAMVWTLDMDDVRGTFCGTGPFPLVYVLNDILVRAEFSSTSLPQFWLSSAVNSSSTDPERLAVTTAWTTDSKILPPGGEAGVTEIHGKCENMTITPRGTTVTPTKETVSLGKHTVALGEKTEITGAMTMTSVGHQSMTPGEKALTPVGHQSVTTGQKTLTSVGYQSVTPGEKTLTPVGHQSVTPVSHQSVSPGGTTMTPVHFQTETLRQNTVAPRRKAVAREKVTVPSRNISVTPEGQTMPLRGENLTSEVGTHPRMGNLGLQMEAENRMMLSSSPVIQLPEQTPLAFDNRFVPIYGNHSSVNSVTPQTSPLSLKKEIPENSAVDEEA

SEQ ID NO: 8 - protein, 721 aa, Mus musculus
MGRLLLLAGLVLLMKHSDGTAYKLVCYFTNWAHSRPGPASIMPHDLDPFLCTHLIFAFASMSNNQIVAKNLQDENVLYPEFNKLKERNRELKTLLSIGGWNFGTSRFTAMLSTLANREKFIDSVISFLRIHGFDGLDLFFLYPGLRGSPPHDRWNFLFLIEELQFAFEREALLTQHPRLLLSAAVSGIPSIIHTSYDALLLGRRLDFINVLSYDLHGSWEKFTGHNSPLFSLPEDSKSSAYAMNYWRKLGTPADKLIMGFPTYGRNFYLLKESKNGLQTASMGPASPGKYTKQAGFLAYYEVCSFVQRAKKHWIDYQYVPYAFKGKEWLGYDDTISFSYKAMYVKREHFGGAMVWTLDMDDVRGTFCGNGPFPLVHILNELLVQTESNSTPLPQFWFTSSVNASGPGSENTALTEVLTTDTIKILPPGGEAMTTEVHRRYENMTTVPSDGSVTPGGTASPRKHAVTPENNTMAAEAKTMSTLDFFSKTTTGVSKTTTGISKTTTGVSKTTTGVSKATAGISKTIPEISKATAGVSKTTTGVSKTTTGISKTITGVSKTTTGISKTTTGISKTTTGVSKITTGVSKTTTGISKTTTGISQTTTGISKTTTDISKTTTGISKTTPGISKTTPGMTVIVQTQANEAETTATMDHQSVTPTEMDTTLFYLKTMTPSEKETSRKKTMVLEKATVSPREMSATPNGQSKTLKWASLITEVETYSQDG

SEQ ID NO: 9 - protein, 368 aa, Homo sapiens
MVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNAYLNVDYVMNYWKDNGAPAEKLIVGFPTYGHNFILSNPSNTGIGAPTSGAGPAGPYAKESGIWAYYEICTFLKNGATQGWDAPQEVPYAYQGNVWVGYDNVKSFDIKAQWLKHNKFGGAMVWAIDLDDFTGTFCNQGKFPLISTLKKALGLQSASCTAPAQPIEPITAAPSGSGNGSGSSSSGGSSGGSGFCAGRANGLYPVANNRNAFWHCVNGVTYQQNCQAGLVFDTSCDCCNWA

SEQ ID NO: 10 - protein, 315 aa, Homo sapiens
MREAFEQEAKQINKPRLMVTAAVAAGISNIQSGYEIPQLSQYLDYIHVMTYDLHGSWEGYTGENSPLYKYPTDTGSNAYLNVDYVMNYWKDNGAPAEKLIVGFPTYGHNFILSNPSNTGIGAPTSGAGPAGPYAKESGIWAYYEICTFLKNGATQGWDAPQEVPYAYQGNVWVGYDNVKSFDIKAQWLKHNKFGGAMVWAIDLDDFTGTFCNQGKFPLISTLKKALGLQSASCTAPAQPIEPITAAPSGSGNGSGSSSSGGSSGGSGFCAGRANGLYPVANNRNAFWHCVNGVTYQQNCQAGLVFDTSCDCCNWA

SEQ ID NO: 11 - protein, 398 aa, Mus musculus
MAKLILVTGLAILLNVQLGSSYQLMCYYTSWAKDRPIEGSFKPGNIDPCLCTHLIYAFAGMQNNEITYTHEQDLRDYEALNGLKDKNTELKTLLAIGGWKFGPAPFSAMVSTPQNRQIFIQSVIRFLRQYNFDGLNLDWQYPGSRGSPPKDKHLFSVLVKEMRKAFEEESVEKDIPRLLLTSTGAGIIDVIKSGYKIPELSQSLDYIQVMTYDLHDPKDGYTGENSPLYKSPYDIGKSADLNVDSIISYWKDHGAASEKLIVGFPAYGHTFILSDPSKTGIGAPTISTGPPGKYTDESGLLAYYEVCTFLNEGATEVWDAPQEVPYAYQGNEWVGYDNVRSFKLKAQWLKDNNLGGAVVWPLDMDDFSGSFCHQRHFPLTSTLKGDLNIHSASCKGPY

SEQ ID NO: 12 - protein, 402 aa, Mus musculus
MAKLILVTGLAILLNVQLGSSYQLMCYYTSWAKDRPTEGSFKPGNIDPCLCTHLIYAFAGMKNNEITYLSEQDLRDYEALNGLKDRNTELKTLLAIGGWKFGPAPFSSMVSTPQNRQTFIKSVIRFLRQYNFDGLNLDWQYPGSRGSPPKDKHLFSVLVQEMRKAFEEESTLNHIPRLLLTSTGAGFIDVIKSGYKIPELSQSLDYIQVMTYDLHDPKNGYTGENSPLYKSPYDIGKSADLNVDSIITYWKDHGAASEKLIVGFPAYGHTFILSDPSKNGIGDPTVSAGPPGKYTNEQGLLAYFEICTFLNEGATEIFDATQEVPYAYLGNEWVGYDNVRSFKLKAQWLKDNNLGGAVVWPLDMDDFSGSFCHQGRFPLTTTLKRDLNVHSASCKASYRGEL

SEQ ID NO: 13 - protein, 390 aa, Homo sapiens
MGATTMDQKSLWAGVVVLLLLQGGSAYKLVCYFTNWSQDRQEPGKFTPENIDPFLCSHLIYSFASIENNKVIIKDKSEVMLYQTINSLKTKNPKLKILLSIGGYLFGSKGFHPMVDSSTSRLEFINSIILFLRNHNFDGLDVSWIYPDQKENTHFTVLIHELAEAFQKDFTKSTKERLLLTAGVSAGRQMIDNSYQVEKLAKDLDFINLLSFDFHGSWEKPLITGHNSPLSKGWQDRGPSSYYNVEYAVGYWIHKGMPSEKVVMGIPTYGHSFTLASAETTVGAPASGPGAAGPITESSGFLAYYEICQFLKGAKITRLQDQQVPYAVKGNQWVGYDDVKSMETKVQFLKNLNLGGAMIWSIDMDDFTGKSCNQGPYPLVQAVKRSLGSL

SEQ ID NO: 14 - protein, 393 aa, Homo sapiens
MRTLFNLLWLALACSPVHTTLSKSDAKKAASKTLLEKSQFSDKPVQDRGLVVTDLKAESVVLEHRSYCSAKARDRHFAGDVLGYVTPWNSHGYDVTKVFGSKFTQISPVWLQLKRRGREMFEVTGLHDVDQGWMRAVRKHAKGLHIVPRLLFEDWTYDDFRNVLDSEDEIEELSKTVVQVAKNQHFDGFVVEVWNQLLSQKRVGLIHMLTHLAEALHQARLLALLVIPPAITPGTDQLGMFTHKEFEQLAPVLDGFSLMTYDYSTAHQPGPNAPLSWVRACVQVLDPKSKWRSKILLGLNFYGMDYATSKDAREPVVGARYIQTLKDHRPRMVWDSQASEHFFEYKKSRSGRHVVFYPTLKSLQVRLELARELGVGVSIWELGQGLDYFYDLL

SEQ ID NO: 15 - DNA, 321 bp, Homo sapiens
gacatccagatgacccagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtatggtagctcactctggacgttcggccaagggaccaaggtggaaatcaaa

SEQ ID NO: 16 - protein, 107 aa, Homo sapiens
DIQMTQSPATLSVSPGERATLSCRASQSVSSNLAWYQQKPGQAPRLLIYGASTRATGIPARFSGSGSGTEFTLTISSLQSEDFAVYYCQQYGSSLWTFGQGTKVEIK

SEQ ID NO: 17 - DNA, 366 bp, Homo sapiens
gaagttcaattgttagagtctggtggcggtcttgttcagcctggtggttctttacgtctttcttgcgctgcttccggattcactttctctacttacggtatggattgggttcgccaagctcctggtaaaggtttggagtgggtttcttatatctctccttctggtggctatactatgtatgctgactccgttaaaggtcgcttcactatctctagagacaactctaagaatactctctacttgcagatgaacagcttaagggctgaggacacggctgtgtattactgtgcgcgacattccccccggagcgcgggagccaatgcttttgatatctggggccaagggacaatggtcaccgtctcaagc

SEQ ID NO: 18 - protein, 122 aa, Homo sapiens
EVQLLESGGGLVQPGGSLRLSCAASGFTFSTYGMDWVRQAPGKGLEWVSYISPSGGYTMYADSVKGRFTISRDNSKNTLYLQMNSLRAEDTAVYYCARHSPRSAGANAFDIWGQGTMVTVSS

SEQ ID NO: 19 - DNA, 336 bp, Homo sapiens
gacatccagatgacccagtctccactctccctgtccgtcagcgttggacagtcggcctccatctcctgcaggtccagtcacagcctcctatacagtgatgggaatacctacataaattggtttcaccagaggccaggccaatctcccaggcgcctaattcatatggtttctaagcgggactctggggtcccagacagattcagcggcagtgggtctggcactgatttcacactgaaaatcagcagggcggaggctgaggatgttggggtttattattgcatgcaagctacatacttcccgctcactttcggcggagggaccagggtggagatcaaa

SEQ ID NO: 20 - protein, 112 aa, Homo sapiens
DIQMTQSPLSLSVSVGQSASISCRSSHSLLYSDGNTYINWFHQRPGQSPRRLIHMVSKRDSGVPDRFSGSGSGTDFTLKISRAEAEDVGVYYCMQATYFPLTFGGGTRVEIK

SEQ ID NO: 21 - DNA, 354 bp, Homo sapiens
gaagttcaattgttagagtctggtggcggtcttgttcagcctggtggttctttacgtctttcttgcgctgcttccggattcactttctctaagtaccttatgcagtgggttcgccaagctcctggtaaaggtttggagtgggtttcttctatcgttccttctggtggcgagactgtttatgctgactccgttaaaggtcgcttcactatctctagagacaactctaagaatactctctacttgcagatgaacagcttaagggctgaggacacggccgtgtattactgtgcgagaagtgggacctatggggcttcgcggtactggggccagggaaccctggtcaccgtctcaagc

SEQ ID NO: 22 - protein, 118 aa, Homo sapiens
EVQLLESGGGLVQPGGSLRLSCAASGFTFSKYLMQWVRQAPGKGLEWVSSIVPSGGETVYADSVKGRFTISRDNSKNTLYLQMNSLRAEDTAVYYCARSGTYGASRYWGQGTLVTVSS

SEQ ID NO: 23 - DNA, 318 bp, Homo sapiens
gacatccagatgacccagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactcccccactttcggcggagggaccaaggtggagatcaaa

SEQ ID NO: 24 - protein, 106 aa, Homo sapiens
DIQMTQSPATLSLSPGERATLSCRASQSVSSYLAWYQQKPGQAPRLLIYDASNRATGIPARFSGSGSGTDFTLTISSLEPEDFAVYYCQQRSNSPTFGGGTKVEIK

SEQ ID NO: 25 - DNA, 360 bp, Homo sapiens
gaagttcaattgttagagtctggtggcggtcttgttcagcctggtggttctttacgtctttcttgcgctgcttccggattcactttctctttttaccgtatggcttgggttcgccaagctcctggtaaaggtttggagtgggtttctcgtatctcttcttctggtggccctactatgtatgctgactccgttaaaggtcgcttcactatctctagagacaactctaagaatactctctacttgcagatgaacagcttaagggctgaggacacagccgtgtattactgtaccacagaaaaacaatacgccggtgtcacttttgagtcctggggccagggaaccctggtcaccgtctcaagc

SEQ ID NO: 26 - protein, 120 aa, Homo sapiens
EVQLLESGGGLVQPGGSLRLSCAASGFTFSFYRMAWVRQAPGKGLEWVSRISSSGGPTMYADSVKGRFTISRDNSKNTLYLQMNSLRAEDTAVYYCTTEKQYAGVTFESWGQGTLVTVSS

SEQ ID NO: 27 - DNA, 330 bp, Homo sapiens
cagagcgctttgactcagccaccctcagcgtctgggacccccggacagagggtcaccatctcttgttctggaagcggctccaacatcggaagtaatactgtaaactggtaccagcagctcccaggaacggcccccaaactcctcatctatagtagtaatcagcggccctcaggggtctctgaccgattctctggctccaagtctgtcgcctcagcctccctggccatcaatggactccagtctgaggatgaggctgattattactgtgcagggtgggatgacagcctgaatggtccggtgttcggcggagggaccaaactgaccgtcctg

SEQ ID NO: 28 - protein, 110 aa, Homo sapiens
QSALTQPPSASGTPGQRVTISCSGSGSNIGSNTVNWYQQLPGTAPKLLIYSSNQRPSGVSDRFSGSKSVASASLAINGLQSEDEADYYCAGWDDSLNGPVFGGGTKLTVL

SEQ ID NO: 29 - DNA, 369 bp, Homo sapiens
gaagttcaattgttagagtctggtggcggtcttgttcagcctggtggttctttacgtctttcttgcgctgcttccggattcactttctctccttaccttatgtggtgggttcgccaagctcctggtaaaggtttggagtgggtttcttatatctcttcttctggtggctatacttcttatgctgactccgttaaaggtcgcttcactatctctagagacaactctaagaatactctctacttgcagatgaacagcttaagggctgaggacacagccatgtattactgtgcacggatgtattactatgatagtagtggttacccctcatttgactactggggccagggaaccctggtcaccgtctcaagc

SEQ ID NO: 30 - protein, 123 aa, Homo sapiens
EVQLLESGGGLVQPGGSLRLSCAASGFTFSPYLMWWVRQAPGKGLEWVSYISSSGGYTSYADSVKGRFTISRDNSKNTLYLQMNSLRAEDTAMYYCARMYYYDSSGYPSFDYWGQGTLVTVSS

SEQ ID NO: 31 - DNA, 31 bp, Artificial
  synthetic primer
ggatcccaacatggccaagctcattcttgtc

SEQ ID NO: 32 - DNA, 44 bp, Artificial
  synthetic primer
gaattctaatggtgatggtgatggtgataagggcccttgcaact

SEQ ID NO: 33 - DNA, 31 bp, Artificial; identical to SEQ ID NO: 31
  synthetic primer

SEQ ID NO: 34 - DNA, 47 bp, Artificial
  synthetic primer
gaattctagtgatggtgatggtgatgaagctcccctcgataagaggc

SEQ ID NO: 35 - DNA, 31 bp, Artificial
  synthetic primer
ggatcccaccatggccaagctacttctcgtc

SEQ ID NO: 36 - DNA, 44 bp, Artificial
  synthetic primer
gaattctagtgatggtgatggtgatgtggccagttgcagcaatt

SEQ ID NO: 37 - DNA, 31 bp, Artificial
  synthetic primer
agatctcaccatggtgcagtccctggcctgg

SEQ ID NO: 38 - DNA, 44 bp, Artificial
  synthetic primer
gaattctagtgatggtgatggtgatggactggagttggatgggg

SEQ ID NO: 39 - DNA, 30 bp, Artificial
  synthetic primer
ggatccaccatgggcatgagggcggcactg

SEQ ID NO: 40 - DNA, 45 bp, Artificial
  synthetic primer
tctagactagtgatggtgatggtgatgagccagggcatccttgat

SEQ ID NO: 41 - DNA, 33 bp, Artificial
  synthetic primer
ggatcccaccatgacaaagcttattctcctcac

SEQ ID NO: 42 - DNA, 45 bp, Artificial
  synthetic primer
gaattcttagtgatggtgatggtgatgtgcccagttgcagcaatc

SEQ ID NO: 43 - DNA, 33 bp, Artificial
  synthetic primer
aagcttcaccatggtgcggtctgtggcctgggc

SEQ ID NO: 44 - DNA, 45 bp, Artificial
  synthetic primer
ggatcctagtgatggtgatggtgatgattccaggtgcagcatttg

SEQ ID NO: 45 - DNA, 31 bp, Artificial
  synthetic primer
aagcttcaccatgggtgtgaaggcgtctcaa

SEQ ID NO: 46 - DNA, 44 bp, Artificial
  synthetic primer
ggatcctaatggtgatggtgatggtgcgttgcagcgagtgcatc

SEQ ID NO: 47 - DNA, 34 bp, Artificial
  synthetic primer
aaggatccaccatggtttctactcctgagaaccg

SEQ ID NO: 48 - DNA, 45 bp, Artificial; identical to SEQ ID NO: 42
  synthetic primer

SEQ ID NO: 49 - DNA, 31 bp, Artificial
  synthetic primer
aagcttcaccatgggagcaaccaccatggac

SEQ ID NO: 50 - DNA, 44 bp, Artificial; identical to SEQ ID NO: 46
  synthetic primer

SEQ ID NO: 51 - DNA, 18 bp, Artificial
  synthetic primer
tgggagctccaatctcag

SEQ ID NO: 52 - DNA, 29 bp, Artificial
  synthetic primer
aaaagcttctaagccagggcatccttgat

SEQ ID NO: 53 - DNA, 8 bp, Artificial
  synthetic primer
ggccagct

(19) United States
(12) Patent Application Publication
(10) Pub. No.: US 2011/0059100 A1
(43) Pub. Date: Mar. 10, 2011

(54) C/CLP Antagonists And Methods Of Use Thereof

(75) Inventors: Jennifer L. Reed, Clarksburg, MD (US); Wendy I. White, Germantown, MD (US); Anthony Coyle, Washington, DC (US); Alexander Kozhich, Rockville, MD (US); Jack Elias, Woodbridge, CT (US); Nanci Donacki, Thurmont, MD (US); Changshou Gao, Potomac, MD (US); Herren Wu, Boyds, MD (US)

(73) Assignees: MEDIMMUNE, LLC, Gaithersburg, MD (US); YALE UNIVERSITY, New Haven, CT (US)

(21) Appl. No.: 12/351,145

(22) Filed: Jan. 9, 2009

Related U.S. Application Data
(63) Continuation of application No. 11/512,267, filed on Aug. 30, 2006, now abandoned.
(60) Provisional application No. 60/801,379, filed on May 19, 2006.
(60) Provisional application No. 60/712,391, filed on Aug. 31, 2005.

(51) Int. Cl. A61K 39/395; C07K 16/00; A61P 11/00

(52) U.S. Cl. 424/146.1; 530/388.1; 530/387.3

(57) ABSTRACT

The invention relates to the novel discovery that antagonizing a C/CLP can be useful for the treatment of diseases associated with the upregulation of one or more C/CLP such as Th2-driven and/or IL-13 mediated inflammatory diseases. Accordingly the present invention provides C/CLP antagonists and also provides compositions and methods for the prevention, management, treatment or amelioration of an inflammatory condition associated with the upregulation of a C/CLP or one or more symptoms thereof and/or the inhibition of IL-13 mediated inflammation.

### 1. CROSS REFERENCE TO RELATED APPLICATIONS

This application claims the benefit under 35 U.S.C. §119(e) of the following U.S. Provisional Application No. 60/712,391, filed Aug. 31, 2005 and 60/801,379, filed May 19, 2006. The priority applications are hereby incorporated by reference herein in their entirety for all purposes.

### 2. BACKGROUND OF THE INVENTION

The prevalence of asthma has been steadily increasing for the past two decades, with an estimated 17 million cases in the United States alone. Asthma is characterized as a complex inflammatory disease attributed to the inappropriate stimulation of the immune system. In some cases, the inflammation is triggered by airborne antigens. In others, exogenous triggers cannot be defined (intrinsic asthma). Immune-mediated inflammation is thought to lead to airway remodeling, or structural modifications, in the asthmatic airway. The end result of remodeling is believed to contribute to both the symptoms and physiological dysregulation of asthma. Remodeling is often characterized by airway thickening, mucus metaplasia, epithelial hypertrophy and airway fibrosis. Extensive fibrosis is widely considered to increase disease severity, airway hyperresponsiveness (AHR) and contribute to the generation of incompletely reversible airway obstruction (Elias et al., 1999, J. Clin. Invest. 104:1001-1006). Therefore, the successful design of therapeutics for the treatment of asthma requires an understanding of both the mechanisms of inflammation and the processes of injury and wound healing in the respiratory system.

Chronic obstructive pulmonary disease (COPD, clinically defined as chronic bronchitis, emphysema, and chronic obstructive lung disease) has long been thought of as a disease distinct from asthma. However, the similarities between the two diseases have been noted and have resulted in the formulation of the “Dutch Hypothesis”, that was first proposed in 1961. The most recent revision of the Dutch Hypothesis proposes that asthma and COPD, in some individuals, are not distinct processes, and that common pathogenic mechanism underlie these disorders. The hypothesis further states that a genetic predisposition to develop atopy, asthma, AHR and/or increased levels of IgE predispose cigarette smokers to develop COPD (Vestbo and Prescott, 1998, Thorax 53:515-519).

The immune cells and mediators implicated in asthmatic inflammation include IgE, mast cells, eosinophils, T cells, interleukin-4 (IL-4), IL-5, IL-9, IL-13 and other cytokines (see, e.g., Bedding et al., 1994, Am. J. Respir. Cell Mol. Biol. 10:471-480; Bradding et al., 1997, Airway Wall Remodeling in Asthma, CRC Press, Boca Raton, Fla.; Nicolaides et al., 1997, Proc. Natl. Acad. Sci. USA 94:13175-13180; Wills-Karp, 1998, Science 282:2258-2260; 20 Hamid et al., 1991, J. Clin. Invest. 87:1541-1546; Kotsimbos et al., 1996, Proc. Assoc. Am. Physicians 108:368-373). Of these immune cells and mediators, the role of T-helper type 2 (Th2) cells and cytokines, are proving to be increasingly important, as they are believed to be responsible for initiation and maintenance of airway inflammation, as well as vital to B cell regulation, eosinophil function, mucus responses, and stimulation of airway remodeling (Elias et al., 1999, J. Clin. Invest. 104:1001-1006; Ray et al., 1999, J. Clin. Invest. 104:985 993). The critical role of IL-13 in many of the symptoms of asthma has been demonstrated (Wills-Karp (1998) Science 282:2258-2260, Grunig et al. (1998) Science 282:2261-2263, Zheng et al. (1999) J. Clin. Invest. 103:779-788) and polymorphisms in both the IL-13 promoter and the coding region have been associated with the asthmatic phenotype (Heinzmann et al. (2000) Hum. Mol. Genet. 9:549-559). These results suggest that abnormal IL-13 production is a critical component of asthmatic inflammation and airway remodeling.

Another family of proteins recently implicated in the physiology of asthma and other inflammatory diseases is the family of chitinase and chitinase-like proteins (see US 20030049261; Zhu et al., 2004, Science 304:1678-1682). Chitin, a polysaccharide made up of chains of N-acetyl-D-glucosamine (GlcNAc), is second only to cellulose in biomass and is an important component of cell walls. Several families of enzymes of distinctly different structures can hydrolyze chitin, including the glycohydrolase family 18 chitinases. This family has representatives among bacteria, fungi, and higher plants (Robertus and Monzingo (1999) EXS. 87: 125-35). Chitinases have also recently been identified in mammals, including chitotriosidase, which is expressed in phagocytes, and AMCase (acidic mammalian chitinase), which is abundant in the gastrointestinal tract (Boot et al. (2001) J. Biol. Chem. 276(9): 6770-6778).

Mammals also express genes coding for proteins that show significant amino acid sequence similarity to chitinases of family 18 glycosyl hydrolases that have been dubbed “chitinase-like” proteins. These proteins have no chitinase activity due to changes in critical amino acid residues in the putative active center, and are thought to have a general function in morphogenesis (Bleau et al. (1999) EXS. 87: 211-21). One of these chitinase-like proteins is oviductin, which is most likely involved in fertilization and protection of the tubal epithelium (Malette et al. (1995) Mol. Reprod. Dev. 41(3): 384-97). Another is YKL-40 (HCgp39), which is produced in association with tissue remodeling (Volck et al. (1998) Proc. Assoc. Am. Physicians. 110(4): 351-60) and other diseases including meningitis (Ostergaard et al (2002) Clin Diagn Lab Immunol 9:598-604), joint injury/arthritis (Johansen et al. (1996) Br J Rheumatol 35:553-9) and cancers (Johansen et al (2004) Lung Cancer 46:333-40).

In US 20030049261, the inventors reported that both chitinase and chitinase-like proteins, as well as the mRNAs encoding them, are present in increased levels in inflammatory disease tissue as compared with the level of such molecules in normal tissue. Further, the inventors found that administration of chitinase protein inhibitors in a model of inflammatory disease associated with, or mediated by, expression of IL-13, treated the disease. Since the filing of US 20030049261, another group has reported an association of the chitinase-like protein Ym-1 with allergic lung inflammation (Webb et al. (2001) J. Biol. Chem. 276(45): 41969-76), while another has observed upregulation of gene expression for several members of the chitinase/chitinase-like protein family in IL-13 deficient, Th2 polarized mice (Sandler et al. (2003) J. Immunol. 171: 1655-67). Accordingly, it has been suggested that the chitinases may play a role in the innate immune response to microbial pathogens.

Chitinase/chitinase-like protein gene expression has also been linked to other types of inflammatory diseases. For instance, rheumatoid arthritis patients demonstrating a remission of active disease showed a significant decrease in serum YKL-40, while patients who changed from inactive to active disease showed an increase in serum YKL-40 (Johansen et al. (1999) Rheumatology (Oxford) 38(7): 618-26). In other studies, serum and synovial fluid levels of YKL 40 were correlated to the severity of the disease for rheumatoid arthritis, osteoarthritis and ankylosing spondylitis (Morgante et al. (1999) Minerva Med. 90(11-12): 437-41; Morgante et al. (2001) Minerva Med. 92(3): 151-3; D'Amore et al. (2000) Minerva Med. 91(3-4): 59-68). Still, however, the role of chitinase and chitinase-like proteins in inflammation is not completely understood, with one group suggesting a physiological role for YKL-40 in limiting the catabolic effects of inflammatory cytokines in patients with inflammatory arthritis (Ling and Recklies (2004) Biochem. J. 380(Pt 3): 651-9).

Despite progress in illuminating the underlying mechanisms and causes of asthma, COPD and related pulmonary inflammatory disorders, asthma remains, along with tuberculosis and AIDS, the only chronic disease with an increasing death rate. In addition, by 2020, COPD is expected to be the fourth leading cause of death in the world. The identification of chitinase/chitinase-like proteins as therapeutic targets for the development of new anti-inflammatory therapies, particularly those Th2-driven diseases such as asthma and allergic diseases, including atopic dermatitis and allergic rhinitis has resulted in a need in the art to develop such therapies. Given the fact that antibodies that bind to chitinase/chitinase-like proteins have been shown modulate Th2-driven diseases such as asthma, and the fact that modulating Th2-mediated immune responses in humans may have profound therapeutic uses for many diseases and disorders, there is a particular need in the art to identify antibodies that can modulate the activity of the chitinase/chitinase-like proteins in the development of Th2-driven diseases. Specifically, antibodies that bind and modulate the activities of chitinase/chitinase-like proteins and polypeptide fragments thereof, that have high affinity for chitinase/chitinase-like proteins and low immunogenicity.

All publications and patent applications herein are incorporated by reference to the same extent as if each individual publication or patent application was specifically and individually indicated to be incorporated by reference.

The publications discussed herein are provided solely for their disclosure prior to the filing date of the present application. Nothing herein is to be construed as an admission that the present invention is not entitled to antedate such publication by virtue of prior invention. Citation or discussion of a reference herein shall not be construed as an admission that such is prior art to the present invention.

### 3. SUMMARY OF THE INVENTION

The present invention provides novel molecules, compounds and methods of use the same for the treatment of inflammatory diseases and disorders. The novel compounds antagonize one or more activity of a least one chitinase/chitinase-like protein (C/CLP) of the invention including, but are not limited to, acidic mammalian chitinase (also referred to as ECF-L), Ym1, Ym2, cartilage glycoprotein 1 (also referred to as BRP-39, chitinase 3-like 1, GP-39, YKL-40), chitotriosidase, oviductal glycoprotein 1 (also referred to as mucin 9, oviductin), cartilage glycoprotein-39 (also known as chitinase 3-like 1, GP-39, YKL 40), chondrocyte protein 39 (also known as chitinase 3-like 2, YKL-39), TSA1902-L and TSA1902-S.

The novel C/CLP antagonists (referred to herein as “C/CLP antagonists of the invention” or simply as “C/CLP antagonists”) include any suitable molecule that disrupts one or more activity of a C/CLP of the invention, including antibodies and antibody fragments having specificity for a C/CLP of the invention, and inactive receptor-binding fragments of C/CLPs, i.e., C/CLP fragments that bind a C/CLP-receptor but do not stimulate C/CLP activation, as well as inactive chitinase-binding fragments of a C/CLP receptor, i.e., a receptor fragment that binds a C/CLP but is not activated and soluble C/CLP receptors, i.e., an Fc fusion protein comprising the extracellular domain of a C/CLP receptor.

The C/CLP antagonists employed in the methods of the present invention are, in one embodiment, those that modulate, decrease or inhibit the ability of a C/CLP to mediated inflammatory responses including, but not limited to, those mediated by IL-13, such as the production of MCP-1, MCP-2, MIP-1β, eotaxin, eotaxin-2 and ENA-78, and those mediated directly by a C/CLP, such as production of MCP-1 and eotaxin. In other embodiments, the C/CLP antagonists employed in the methods of the present invention are those that modulate one or more activity of a C/CLP including, but not limited to, chitinolytic activity, saccharide (e.g., GlcNAc) binding activity, galectin binding and/or other protein-protein interactions, modulation of galectin function, chemotatic activity, receptor binding activity, and signal transduction activity.

In one embodiment, a C/CLP antagonist of the invention antagonizes a single C/CLP. In another embodiment, a C/CLP antagonist of the invention antagonizes multiple C/CLPs. In still another embodiment, a C/CLP antagonist of the invention antagonizes a single activity of at least one C/CLP. In yet another embodiment, a C/CLP antagonist of the invention antagonizes more then one activity of at least one C/CLP.

In a specific embodiment, a C/CLP antagonist binds the signal sequence of a C/CLP. In another specific embodiment, a C/CLP antagonist binds the catalytic domain of a C/CLP. In still another specific embodiment, a C/CLP antagonist binds the chitin binding domain of a C/CLP.

In one embodiment, a C/CLP antagonist is a chemical compound. In a specific embodiment, a C/CLP antagonist is a chemical compound known compound including, but not limited to, allosamidin and its derivatives, glucoallosamidin A, glucoallosamidin B, methyl-N demethylallosamidin, demethylallosamidin, and didemthylallosamidin (Zhou et al., 1993, J. Antibiotics 46:1582-1588). Further contemplated chemical C/CLP antagonists include stylogaunidine and its derivatives, dipeptide cyclo-(L-Arg-D-Pro), divalent cations (e.g., Cu 2+, Zn2+′ and Hg2+), and riboflavin and; flavin derivatives.

In another embodiment, a C/CLP antagonist is a nucleic acid molecule. In a specific embodiment a C/CLP antagonist is a nucleic acid molecule that inhibits or reduces the level of messenger RNA encoding a C/CLP including, but not limited to, an aptamer, a ribozyme, an antisense molecule, an siRNA.

In one embodiment, a C/CLP antagonist of the invention is a polypeptide molecule (including, but not limited to, proteins, post-translationally modified proteins, antibodies etc.). In a specific embodiment, a C/CLP antagonist of the invention is an antibody. In another specific embodiment, antibody C/CLP antagonists specifically bind a C/CLP of the invention, for example, AMCase. Also contemplated are antibody C/CLP antagonists that bind more then one C/CLP.

The present invention also provides methods of inhibiting inflammatory responses including, but not limited to, those mediated by IL-13 using the C/CLP antagonists of the invention. In one embodiment, the methods of the invention comprise the administration of an effective amount of one or more C/CLP antagonist alone or in combination with other prophylactic or therapeutic agents. The present invention also provides methods for the prevention, management, treatment and/or amelioration of a disease or disorder associated with aberrant (i.e., increased, decreased or inappropriate) C/CLP expression and/or activity. Such diseases and disorders diseases include, but are not limited to, interstitial lung disease (ILD), pulmonary fibrosis, bronchitis, chronic obstructive pulmonary disease (COPD), pneumonia, pneumonitis, acute respiratory distress syndrome (ARDS), severe acute respiratory syndrome (SARS), sarcoidosis, cystic fibrosis (CF), emphysema, asthma, smoker's cough, allergy, allergic rhinitis, sinusitis, Paget's disease, abnormal bone remodeling, osteoporosis, Gorham-Stout syndrome, osteoarthritis, rheumatoid arthritis, psoriatic arthritis and brittle bone disease.

### 4. BRIEF DESCRIPTION OF THE FIGURES

FIG. 1. Structural Features of the Chitinase, Chitinase-Like and Galectin Proteins. The major structural features of the C/CLPs are shown in Panel A. The specked box indicates the signal sequences, designated “SS”. The catalytic domain is represented by the shaded box, the point mutations present in the chitinase-like proteins are indicated by black lines. The serine/glycine rich “hinge” and chitin binding domain (designated CBD) regions present only in the chitinase proteins are indicated by the white and dotted boxes, respectively. See FIG. 2 for analysis of the amino acid sequence homology of several C/CLPs. The conserved primary structures within the CRD in galectin family members are shown in Panel B. Italicized letters indicate those highly conserved residues known to make contacts with carbohydrate ligands. Nonconserved amino acids are indicated by -x- (adapted from, Essentials of Glycobiology, Ajit Varki, et al., eds., Cold Spring Harbor Laboratory Press (NY, 1999) chapter 27).

FIG. 2. Amino Acid Analysis of the human C/CLP Family Members. Panels A-B are an alignment of AMCase with other members of the human C/CLP family, TSA1902 (a splice variant of AMCase), chitotriosidase, YKL-39 and YKL-40 starting with the first residue of the initial translation product. Boxed amino acids are those which are identical to the consensus sequence of the aligned proteins. The amino acids marked with an asterisk are the required catalytic residues of the chitinase DXDXE consensus. The solid line indicated the signal sequence, the dotted line indicates the hinge region of AMCase, the double line indicates the chitin-binding domain.

FIG. 3. Alteration of Key Amino Acids In Catalytic Domain Destroys Enzymatic Activity. The activity of several recombinant AMCase proteins was measured using the 4-Mu-Chitotrioside assay. Both the full length AMCase and the AMCase protein lacking the CBD (designated AMCase-TR) have high activity. The AMCase-TR routinely had slightly higher activity then the full length protein. The AMCase full length proteins containing either the D136A or E140A mutation had no activity.

FIG. 4. mYKL-40 Up Regulates Cytokine Release From Peritoneal Macrophages And Is Inhibited by anti-mYKL-40 Antibodies. Panel A) Peritoneal macrophages treated with increasing amounts of recombinant mouseYKL-40 show a dose sensitive increase in the secretion of both IL-6 (left panel) and KC (right panel). Panel B) Three different monoclonal anti-mYKL-40 antibodies (1903, 102 and 204) inhibited the YKL-40 stimulated release of: KC by ˜56% (top left); TNF-α by ˜70% (top right); RANTES by ˜75% (bottom left); and IL-10 by ˜75% (bottom right).

FIG. 5. Neutralizing Activity and Specificity of Rabbit Polyclonal anti-AMCase and anti-ChT antibodies. The activity of recombinant AMCase and ChT in the presence of either anti-AMCase or anti-ChT antibodies was examined using the 4-Mu-Chitotrioside assay. AMCase activity was inhibited only by the anti-AMCase antibody (#206). Likewise, ChT activity was inhibited only by the anti-ChT antibody (#208).

FIG. 6. Fully Human anti-AMCase Antibodies. The nucleotide and corresponding amino acid sequences (SEQ ID NOS. indicated and listed below) of the variable domain of several fully human anti-AMCase antibodies are show. Panel A shows the variable region of the light chain (top, SEQ ID NOS: 15 and 16) and heavy chain (bottom, SEQ ID NOS: 17 and 18) of M1. Panel B shows the variable region of the light chain (top, SEQ ID NOS: 19 and 20) and heavy chain (bottom, SEQ ID NOS: 21 and 22) of M5. Panel C shows the variable region of the light chain (top, SEQ ID NOS: 23 and 24) and heavy chain (bottom, SEQ ID NOS: 25 and 26) of Z1. Panel D shows the variable region of the light chain (top, SEQ ID NOS: 27 and 28) and heavy chain (bottom, SEQ ID NOS: 29 and 30) of Z8.

FIG. 7. Competition Map of anti-AMCase Antibodies. The antibodies listed in Table 2 were tested for their ability to compete with one another for binding to either human AMCase or mouse AMCase by BIAcore analysis. Those antibodies that compete with each other are indicated by overlapping circles. The brackets indicate those antibodies which do NOT bind to the truncated form of AMCase (Z8, M1, 171.204 and Z4). As indicated in Table 2 several antibodies bind to both mouse and human AMCase. Five distinct binding sites have been identified on mouse AMCase and four on human AMCase. NOTE: only the inter-relatedness of the different epitopes is indicated, NOT the physical location.

FIG. 8 Inhibitory Activity of the Monoclonal Antibodies Assayed Using the Chitin Azure Assay. Panel A shows the activity of mouse AMCase in the presence of several anti-AMCase antibodies (171.204, 4F8, Z8, M1 and M5) using the Chitin Azure Assay. 4F8 was shown to be a potent inhibitor, M1 and 171.204, were shown to have some inhibitory activity while 171.204 did not inhibit in this assay. Panel B shows activity of mouse AMCase in the presence of either the 4F8 antibody or a molar excess of the small molecule inhibitor, allosamidin; VNR140-116 is a negative control antibody. 4F8 was able to inhibit the enzymatic activity of mouse AMCase to the same degree seen for allosamidin.

FIG. 9. Anti-AMCase Staining of Human Asthmatic Lung. Sections of human asthmatic lung were stained with the anti-AMCase antibody Z8, right panel and a control antibody, left panel. Macrophages and Bronchiolar epithelium are indicated by arrows.

FIG. 10. Increased Chitinolytic Activity Detected in Lung Homogenate After Antigen Challenge. Mice were sensitized to the antigen ovalbumin were subsequently challenged intranasally with the same antigen. Lung homogenates obtained 4 hrs post antigen challenge were assayed using the 4-Mu-Chitotrioside assay. The chitinolytic activity in the homogenates increases upon subsequent challenges with the highest level seen after three antigen challenges.

FIG. 11. Chitinolytic Activity in Lung Homogenates is AMCase not ChT. The chitinolytic activity of lung homogenates from antigen challenged mice (see FIG. 10) was assayed in the presence of either anti-AMCase or anti-ChT antibodies using the 4-Mu-Chitotrioside assay. The anti-AMCase antibody inhibited the activity of the homogenates by an average of about 85%, indicated that the vast majority of the chitinolytic activity in the lung homogenates is due to AMCase.

FIG. 12. Inhibition of Cellular Infiltration in Ovalbumin-Induced Asthma by Monoclonal Anti-AMCase Antibodies. Several anti-AMCase antibodies were tested for their ability to inhibit the cellular infiltration of the lung seen in the ovalbumin-induced asthma model in mice. 171.208 and, to a slightly less extent, Z8 were both able to reduce cellular infiltrate, compared to a control antibody (1A7).

FIG. 13. Inhibition of Cellular Infiltration in Cockroach Antigen-Induced Asthma by Monoclonal Anti-AMCase Antibodies. The 171.204 and Z8 antibodies were shown to reduce cellular infiltration in another antigen-induced asthma model, using cockroach antigen which contains chitin. 171.204 was slightly more inhibitory then Z8.

FIG. 14. Anti-AMCase antibodies Inhibit IL-13 Enhanced AHR. The 171.204 and Z8 antibodies were shown to reduce IL-13 enhanced airway hyperresponsiveness upon methacholine challenge as measured by Penh scores.

FIG. 15. Anti-AMCase antibodies, Which Do Not Inhibit IL-13 Enhanced Chitinolytic Activity. The chitinolytic activity, measured as mAMCase equivalents, in lung homogenates (ng/g) and serum (ng/ml) is similar from animals dosed with IL-13 in combination with an anti-AMCase antibody (Z8, 171.204 or 4F8), BSA/PBS or a control antibody (1A7).

FIG. 16. IL-13 Treatment Increases AMCase and YM1 Transcripts in Whole Lung Tissues. The expression levels of AMCase (panel A) and YM-1 (panel B), as determined by RT-PCR rise one to two logs in mice treated with IL-13 as compared to mock treated mice. Four animals in each treatment group were examined.

FIG. 17. AMCase In Alveolar Macrophages. Immunohistochemistry of AMCase in lung tissue show staining of alveolar macrophages in mice sensitized with cockroach antigen (CrAg) and challenged with CR Ag (top middle) and of mice treated with local IL-13 administration (bottom left). Little staining is seen in lungs from animals treated with local BSA instillation (bottom middle) and animals sensitized with CrAg and not challenged or challenged with PBS (top right). Little background staining with an irrelevant primary antibody is seen (bottom right).

FIG. 18. Protein-Protein Interaction Array Profiling. Depicted is the scheme used for the protein interaction array used to identify Galectin 3 as a binding partner of AMCase.

FIG. 19. Human AMCase Binds Human Galectin 3 and Human Galectin 4. ELISA assay showing that both full length (panel A) and C-terminal truncated (panel B) human AMCase binds human galectin 3 and 4 but not galectin 1, 2, 7 or 8 under the conditions used.

FIG. 20. Lactose Inhibits the Binding of Human AMCase to Both Galectin 3 (solid lines) and Galectin 4 (dashed lines). ELISA assay showing that the binding of Human AMCase to both galectin 3 and 4 is inhibited by lactose (circles) but not allosamindin (squares).

FIG. 21. IL-13 Increases Galectin 3 Transcripts in Whole Lung Tissues. The expression levels of galectin 3 increases about three to four logs in mice treated with IL-13×3 doses as compared to mock treated (NT) mice. Four animals in each treatment group were examined.

FIG. 22. Galectin 3 is Present in Asthmatic Lung. Immunohistochemistry of lung tissue shows galectin 3 staining in mice sensitized with cockroach antigen (CrAg) and challenged with CR Ag (top right) and of mice treated with local IL-13 administration (bottom left). Little staining is seen in lungs from animals sensitized with CrAg and challenged with PBS (top left). Little background staining with an irrelevant primary antibody is seen (bottom right).

FIG. 23. Human Galectin-3 Induces Apoptosis in Jurkat T cells. Left Panel: Jurkat T cells treated with 5, 10 and 30 μg of recombinant human Galectin 3 (rhuGal-3) show increased apoptosis. Little to no apoptosis is seen for untreated cells or those treated with 1 μg of rhuGal-3. Right Panel: The addition of lactose reduced the levels of apoptosis seen to nearly background levels.

FIG. 24. AMCase Inhibits Apoptosis Induced by Galectin 3. Galectin-3 alone induced apoptosis in memory T cells isolated from human blood (solid diamonds). A significant inhibition was seen at the 10 μg dose of galectin-3 by the addition of 50 μg or more of AMCase.

FIG. 25. AMCase and Chitotriosidase (Cht) Inhibit Calcium Flux Induced by Galectin-3. Galectin treatment of Jurkat cells results in calcium flux (line 1). AMCase and Cht at 100 μg/ml inhibit this flux by about 30-40% (lines 2 and 3, respectively). A further inhibition was seen for AMCase at 200 μg/ml (line 4) but not for Cht (line 5).

FIG. 26. AMCase Immunostaining In Controls And Asthmatics. Illustrative immunostaining of AMCase in bronchial biopsies (panels A through E) collected from a control healthy individual (panel A), intermittent asthmatic (panels B, C), moderate asthmatic (panel D) and from severe asthmatic (panel E). AMCase was detected in occasional subepithelial monocyte/macrophages in intermittent asthma and moderate asthma, and strongly present in hyperproliferative mucosal epithelium in severe asthma, with almost no detection in normal tissue.

FIG. 27. ChT Immunostaining In Controls And Asthmatics. Illustrative immunostaining of ChT in bronchial biopsies (panels A through D) collected from a control healthy individual (panel A), an intermittent asthmatic (panel B), and from severe asthmatic (panels C, D). ChTRase was detected in occasional subepithelial monocyte/macrophages in intermittent asthma and moderate asthma, and strongly present in hyperproliferative mucosal epithelium in severe asthma, with almost no detection in normal tissue.

FIG. 28. YKL-40 Immunostaining In Controls And Asthmatics. Illustrative immunostaining of YKL-40 in bronchial biopsies (panels A through E) and in a cytospin preparation from bronchial lavage cells (panel F) collected from a control healthy individual (panel A), a mild asthmatic (panel B), a moderate asthmatic (panel C) and from three distinct severe asthmatics (panels D, E and F). Faint or no expression of YKL-40 is observed in controls (arrow), whereas detectable YKL-40-positive cells are found in subepithelial areas of bronchial biopsies from mild and moderate asthmatics. In severe asthmatics, elevated numbers of YKL-40-bearing cells infiltrating the epithelium are noted (panel D). Some of these patients also exhibit YKL-40 immunostaining on the luminal surface of the bronchial epithelium (panel E). Immunostaining of bronchial lavage cells from severe asthmatics showing the expression of YKL-40 in association with alveolar macrophages and neutrophils (PN, arrows). Scale bars=250 μm (panels A, B and C), 500 μm (panels D and E) and 1,000 μm (panel F).

FIG. 29. Scatter Plot Analysis Of YKL-40-Positive Cells In Bronchial Biopsies From Controls And Asthmatics. There was a statistically significant difference across the four patient groups with the use of Kruskal-Wallis test (p=0.0031). The number of YKL-40-bearing cells was significantly higher in severe, as compared to mild and to moderate asthmatics and to control healthy subjects. Results are expressed as numbers of YKL-40-expressing cells per mm² of bronchial tissue sections. Comparisons among groups were made by the non-parametric Mann-Whitney U tests. The horizontal bars represent median values.

FIG. 30. Inhibition of IL-13 Mediated Airway Responsiveness By Administration of anti-C/CLP antibodies. Pre-treatment with one dose of polyclonal Anti-mYKL-40/BRP-39 (filled squares), Anti AMCase (filled triangles) or Anti YM-1 (filled diamonds) antibody reduced the penh scores by about 40% to 56% (from ˜6.5 to ˜3.2 to 4.2) relative to the control IgG (open diamonds) at the highest methacholine challenge dose of 300 mg/ml. Reduction in penh values were seen for methacholine challenge doses of 30 mg/ml and higher. Pretreatment with a cocktail of all three antibodies (filled circle) did not show any additional decrease in penh score over the administration of a single antibody in these studies.

FIG. 31. Expression of C/CLPs In Mice Infected with Recombinant Adenovirus. Panel A) Relative expression level AMCase increased by 4 to 13 fold in 3 out of 4 mice infected with mouse AMCase expressing adenovirus (Ad-mAMCase, grey bars). No increase in AMCase expression was seen for animals infected Null Adenovirus (Ad-null, white bars) or mYKL-40/BRP-39 expressing adenovirus (Ad-BRP-39, black bars). Panel B) Relative expression level mYKL-40/BRP-39 increased by 2 to 4 fold in 4 mice infected with mYKL-40/BRP-39 expressing adenovirus (Ad-BRP-39, black bars). No increase in mYKL-40/BRP-39 expression was seen for animals infected Null Adenovirus (Ad-null, white bars) or mouse AMCase expressing adenovirus (Ad-mAMCase, grey bars).

FIG. 32. Local Administration of Recombinant Adenovectors, Expressing mYKL-40 Results In A Stronger Lung Phenotype Then Seen For Null or AMCase Adenovectors. Histological analysis of lung tissue from animals treated with either the Null adenovector (right panels) or the AMCase expressing adenovector (left panels) shows mild subacute intra-alveolar mononuclear infiltrate accompanied by occasional neutrophils and a histological score of 1.5, while tissue from 2 out of 4 animals treated with mYKL-40/BRP-39 expressing adenovector (middle panels) shows diffuse alveolitis plus acute to chronic respiratory bronchiolitis with occlusion (serum plus coagulated protein) that extends to larger bronchioles and a histological score of 3.0.

### 5. DETAILED DESCRIPTION

The present inventors have found that antagonists of chitinases and chitinase-like proteins (referred to herein jointly as C/CLP(s)) can be useful for the treatment of diseases associated with the up regulation of one or more C/CLPs such Th2-driven inflammatory diseases of the lung and inflammatory arthritis. The present invention in based in part on the discovery that certain C/CLPs can stimulate the release of cytokines from macrophages in vitro. Furthermore, local administration of certain C/CLPs (e.g., YKL40) in the lung results in alveolitis and chronic respiratory brochiolitis. In addition, the present inventors have also found that certain C/CLPs (e.g., AMCase) can interact with galectins. Furthermore the interaction of C/CLPs with galectins can modulate galectin function.

As described above and further disclosed herein, several members of the C/CLP family are upregulated in a number of inflammatory conditions, in particular Th2 mediated inflammation (see, e.g., Section 6.5, infra). Studies have shown that compounds that antagonize at least one C/CLP ameliorated Th2 inflammation, in part, by inhibiting IL-13 pathway activation and chemokine induction (Zhu et al., 2004, Science 304:1678). The studies disclosed herein, further demonstrated that antagonists of several other C/CLPs reduced IL-13 induced airway hyperresponsiveness (see, e.g., Sections 6.3 and 6.6, infra). Without wishing to be bound by any particular theory, it is likely that C/CLPs do not directly induce Th2 cytokine (e.g., IL-13) production, rather, they function downstream by mediating the effector response of IL-13. This modulation of IL-13 effector signaling enhances Th2-mediated inflammatory diseases and lung diseases. Accordingly, when the ability of the C/CLPs to modulate IL-13 signaling is inhibited, the IL-13 effector responses (e.g., production of MCP-1, MCP-2, MIP-1β, eotaxin, eotaxin-2 and ENA-78) are reduced thereby quelling inflammation associated with Th2-driven IL-13 activity. Furthermore, C/CLPs have been implicated in the pathogenesis of tissue remodeling which may contribute to the development and/or progression of inflammatory and lung diseases.

In addition, as demonstrated herein, at least one C/CLP, acidic mammalian chitinase (AMCase), can interact directly with galectin-3 and galectin-4. The galectins are associated with a variety of extracellular functions including cell motility, metastasis, apoptosis, degranulation, cell adhesion, reduction in IL-5 production by eosinophils and dectin signaling to name a few. In particular, galectin-3 can induce apoptosis and calcium flux in immune cells. AMCase can inhibit both of these galectin-3 medicated cellular responses (see, e.g., Section 6.4, infra). Without wishing to be bound by any particular theory, the interaction of C/CLPs and Galectins may cluster monomeric chitin and/or similar carbohydrate moieties facilitating signaling through cell surface carbohydrate receptors, such as dectin, by inducing cross-linking and activation of the receptors. Alternatively, C/CLPs inhibit the apoptotic activity of galectins which can prolong the inflammatory response in tissues where C/CLPs bind galectins.

Thus, the present invention encompasses methods of inhibiting inflammation associated with Th2-driven IL-13 activity comprising exposing a subject in need thereof, an effective amount of an antagonist of a C/CLP activity such that IL-13 effector responses are inhibited. The present invention also includes methods of decreasing inflammation associated with an increase in the level of one or more C/CLP, particularly lung inflammation, in a patient in need thereof comprising administering an effective amount of an antagonist of a C/CLP. The present invention also includes methods of inhibiting tissue remodeling associated with an inflammatory disease, particularly lung inflammation, in a patient in need thereof comprising administering an effective amount of an antagonist of a C/CLP. Furthermore, the invention also includes methods of preventing and/or reducing the binding of a C/CLP to a galectin and for preventing and/or reducing the inhibitory action of C/CLP on galectin mediated cellular responses.

The inflammatory cells targeted by the present invention are preferably those that are responsive or currently responding to IL-13, wherein a decrease in IL-13 levels via administration of the C/CLP antagonists of the invention leads to decreased inflammation. Alternatively, or in addition, the inflammatory cells targeted by the present invention may contribute to, or be involved in, tissue remodeling. Such cells may be readily identified by the skilled artisan using the methods of the present invention disclosed herein. Such cells may include, among others, lung inflammatory cells involved in diseases including, but not limited to, interstitial lung disease (ILD), pulmonary fibrosis, bronchitis, chronic obstructive pulmonary disease (COPD), pneumonia, pneumonitis, acute respiratory distress syndrome (ARDS), severe acute respiratory syndrome (SARS), sarcoidosis, cystic fibrosis (CF), emphysema, asthma, smoker's cough, allergy, allergic rhinitis and sinusitis. Other cells may include, among others, inflammatory cells involved other chronic inflammatory diseases involving cellular infiltration, tissue damage and remodeling including, but not limited to Paget's disease, abnormal bone remodeling, osteoporosis, Gorham-Stout syndrome, osteoarthritis, rheumatoid arthritis, psoriatic arthritis and brittle bone disease.

The present invention provides C/CLP antagonists, referred to herein as “C/CLP antagonists of the invention” or simply as “C/CLP antagonists.” C/CLP antagonists are employed in the methods of the present invention to effect the inhibition of inflammatory responses including, but not limited to, those mediated by IL-13 or by a C/CLP. IL-13 and/or C/CLP may mediate inflammatory responses directly or indirectly.

Antagonists can include any suitable molecule that disrupts one or more activity of a C/CLP, including antibodies and antibody fragments having specificity for a C/CLP of the invention, and inactive receptor-binding fragments of C/CLPs, i.e., C/CLP fragments that bind a C/CLP-receptor but do not stimulate C/CLP activation, as well as inactive chitinase-binding fragments of a C/CLP receptor, i.e., a receptor fragment that binds a C/CLP but is not activated. Such antagonists may be readily identified by the skilled artisan using the methods of the invention disclosed herein.

The present invention also provides methods to inhibit inflammatory responses including, but not limited to, those mediated by IL-13 or by a C/CLP using the C/CLP antagonists of the invention. In one embodiment, the methods of the invention comprise the administration of an effective amount of one or more C/CLP antagonist alone or in combination with other prophylactic or therapeutic agents. The present invention also provides methods for the prevention, management, treatment and/or amelioration of a disease or disorder associated with aberrant (i.e., increased, decreased or inappropriate) C/CLP expression and/or activity. Such diseases and disorders diseases include, but are not limited to, interstitial lung disease (ILD), pulmonary fibrosis, bronchitis, chronic obstructive pulmonary disease (COPD), pneumonia, pneumonitis, acute respiratory distress syndrome (ARDS), severe acute respiratory syndrome (SARS), sarcoidosis, cystic fibrosis (CF), emphysema, asthma, smoker's cough, allergy, allergic rhinitis, sinusitis, Paget's disease, abnormal bone remodeling, osteoporosis, Gorham-Stout syndrome, osteoarthritis, rheumatoid arthritis, psoriatic arthritis and brittle bone disease.

The C/CLP antagonists employed in the methods of the present invention are, in one embodiment, those that modulate, decrease or inhibit the ability of a C/CLP to mediated inflammatory responses including, but not limited to, those mediated by IL-13, such as the production of MCP-1, MCP-2, MIP-1β, eotaxin, eotaxin-2 and ENA-78, and those mediated directly by a C/CLP, such as production of MCP-1 and eotaxin. In other embodiments, the C/CLP antagonist employed in the methods of the present invention modulate, decrease or inhibit the ability of a C/CLP to mediate tissue remodeling. In other embodiments, the C/CLP antagonists employed in the methods of the present invention are those that modulate one or more activity of a C/CLP including, but not limited to, chitinolytic activity, saccharide (e.g., GlcNAc) binding activity, galectin binding, modulation of galectin-mediated cellular responses, chemotatic activity, receptor binding activity, and signal transduction activity. In a specific embodiment, the C/CLP antagonists employed in the methods of the present invention are those that when administered to a subject in need thereof result in an improvement (e.g., a reduction in severity) in one or more of the changes associated with inflammatory disease including, but not limited to, tissue inflammation, tissue remodeling, increased lung volume, increased eosinophils in bronchioalveolar ravage (BAL) fluid, increased lymphocytes in BAL fluid, increased total cells in BAL fluid, increased alveolus size, increased deposition of crystals comprising chitinase-like molecules in lung tissue, increased airway resistance, increased mucus metaplasia, increased mucin expression, increased parenchymal fibrosis, increased airway remodeling (e.g., airway thickening, mucus metaplasia, epithelial hypertrophy and airway fibrosis), increased subepithelial fibrosis, increased collagen deposition in airway tissue, epithelial hypertrophy in the lung tissue, focal organization of crystalline material into Masson body-like fibrotic foci, airway hyperresponsiveness (AHR), and the like.

The present invention further encompasses isolated antagonists identified according to the disclosed methods and medicaments and therapeutic compositions comprising the same. Isolated antibodies or antigen binding fragments thereof that specifically bind a mammalian chitinase and antagonize the ability of a C/CLP to mediated inflammatory responses, particularly lung inflammation, are included. The antagonists of the present invention may also be administered in combined therapeutic compositions comprising other suitable compounds, particularly one or more inhibitors of the IL-4/IL-13 signal transduction pathway and/or other anti-inflammatory agents.

### 5.1 Galectins

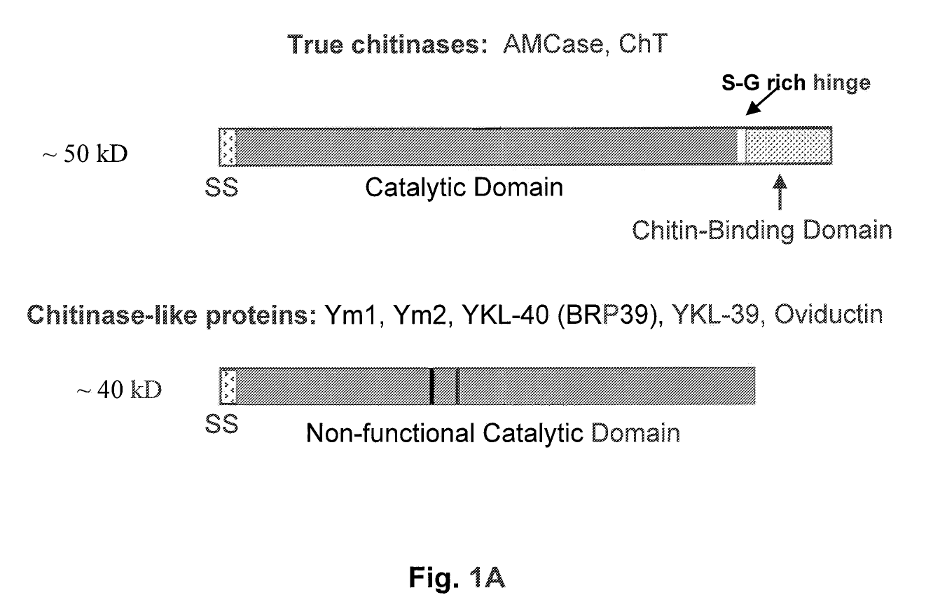

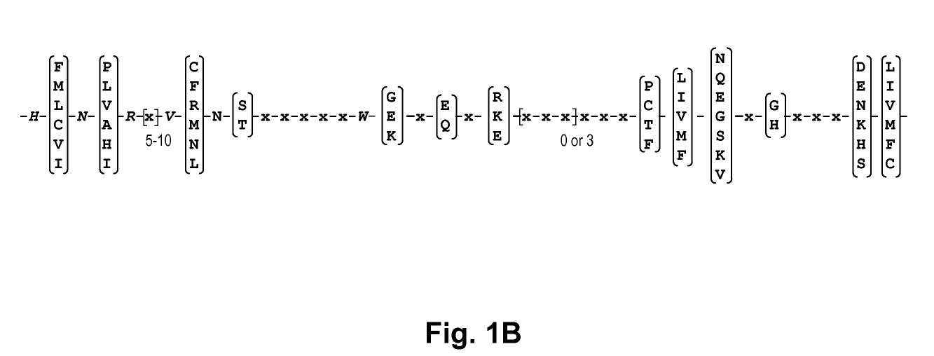

The galectins are a group of proteins that bind β-galactosyl-containing glycoconjugates and share primary structural homology in their carbohydrate recognition domains (CRDs). The CRD is approximately 130 amino acids in length, however a number of studies have indicated that that eight invariant residues are involved in carbohydrate binding. The common sequence motif used to identify galectins, shown in FIG. 1B, is well characterized and described in, for example, Essentials of Glycobiology, Ajit Varki, et al., eds., Cold Spring Harbor Laboratory Press (NY, 1999) chapter 27.

Most galectins are soluble proteins that lack classical signal sequences or membrane-anchoring domains. The galectins appear to be synthesized on free polysomes in the cytoplasm and accumulate there prior to secretion. Many appear to display a requirement for reducing conditions to maintain activity in the absence of ligands. Certain members of the galectin family can promote cell-cell and/or cell-extracellular matrix adhesion and some have potent biological activities, such as the ability to induce apoptosis, or programmed cell death, and to induce metabolic changes, such as cellular activation and mitosis.

The galectins can be divided into two general subgroups, based on sequence homologies. The galectin-1 subfamily, which includes galectins 1 and 2, and the galectin-3 subfamily, which includes all others. At least fourteen galectins (generally referred to as galectin-1 through galectin-14) have now been identified based on the conserved galectin CDR and all appear to recognize simple β-galactosides. Exemplary galectins of the present invention, among others, include galectin-3 (also referred to herein as “gal-3”) and galectin-4 (also referred to herein as “gal-4”) and any other galectin described herein.

Galectins include, but are not limited to galectin-1 (also referred to as Lectin galactoside-binding soluble 1, Beta-galactoside-binding lectin L-14-I, Lactose-binding lectin 1, S-Lac lectin 1, Galactin, 14 kDa lectin, HPL, Putative MAPK-activating protein MP12 and exemplified by GenBank Acc. No. P09382); galectin-2 (also referred to as Beta-galactoside-binding lectin L-14-II, Lactose-binding lectin 2, S-Lac lectin 2, HL14 and exemplified by GenBank Acc. No. P05162); galectin-3 (also referred to as Galactose-specific lectin 3, Mac-2 antigen, IgE-binding protein, 35 kDa lectin, Carbohydrate-binding protein 35, CBP 35, Laminin-binding protein, Lectin L-29 and exemplified by GenBank Acc. No. NP_{—}002297); galectin-4 (also referred to as Lactose-binding lectin 4, L-36 lactose-binding protein, L36LBP, Antigen NY-CO-27 and exemplified by GenBank Acc. No. NP 006140); galectin-5 (exemplified by GenBank Acc. No. AAA65445); galectin-6 (exemplified by GenBank Acc. No. AAC27244); galectin-7 (also referred to as lectin galactoside-binding soluble 7, p53-induced gene 1 and exemplified by GenBank Acc. No. NP_{—}002298); galectin-8 (also referred to as Gal-8, Prostate carcinoma tumor antigen 1, PCTA-1, Po66 carbohydrate-binding protein and exemplified by GenBank Acc. Nos. O00214, NP_{—}963839 and NP_{—}963838); galectin-9 (exemplified by GenBank Acc. Nos. NP_{—}002299 and NP_{—}033665); galectin-10 (exemplified by GenBank Acc. No. AAB04967); galectin-11 (exemplified by GenBank Acc. Nos. AAK68382 and AAO91722P); galectin-12 (also referred to as galactoside-binding, soluble, 12 and exemplified by GenBank Acc. No. NP_{—}149092); galectin-13 (also referred to as Galactoside-binding soluble lectin 13, Placental tissue protein 13, Placenta protein 13, PP13 and exemplified by GenBank Acc. No. NP_{—}002296); galectin-14 (exemplified by GenBank Acc. No. AAL37895).

Specifically included in the invention are the full-length galectin polypeptides, galectin polynucleotides which encode a galectin, and fragments thereof In addition, galectin polynucleotides include polynucleotides sharing at least 30% identity, or at least 40% identity, or at least 50% identity, or at least 60% identity, or at least 70% identity, or at least 80% identity, or at least 90% identity, or at least 95% identity, or at least 98% identity, or at least 99% identity to a polynucleotide which encodes a galectin (or the complement thereof). Methods for determining the percent identity between two sequences are known to one of skill in the art and specific methods are disclosed herein (see, Section 5.2, infra).

Specifically included in the invention are galectin fragments. The term “galectin fragment” and similar terms, include polypeptides comprising, or alternatively consisting of (or consisting essentially of) an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, or at least contiguous 80 amino acid residues of a galectin polypeptide. In certain embodiments the galectin fragment comprises the CRD or a fragment thereof. In other embodiments the galectin fragment comprises that portion of galectin which interacts directly with a C/CLP. One of skill in the art can determine which portion(s) of a galectin interact with C/CLP using one of many known methods in the art. See, e.g., Section 6.4 infra.

### 5.2 Chitinase and Chitinase-Like Molecules of the Invention

“Chitinase,” as used herein, refers to any of the glycohydrolase family 18 chitinases comprising microbial, plant and mammalian chitinases. A “chitinase” of the present invention demonstrates detectable chitinase activity, in that it specifically cleaves chitin, a polysaccharide made up of chains of N-acetyl-D-glucosamine (GlcNAc), in a chitinolytic manner. Preferred chitinases of the present invention, among others, include acidic mammalian chitinase (AMCase) and chitotriosidase and any other chitinase described herein.

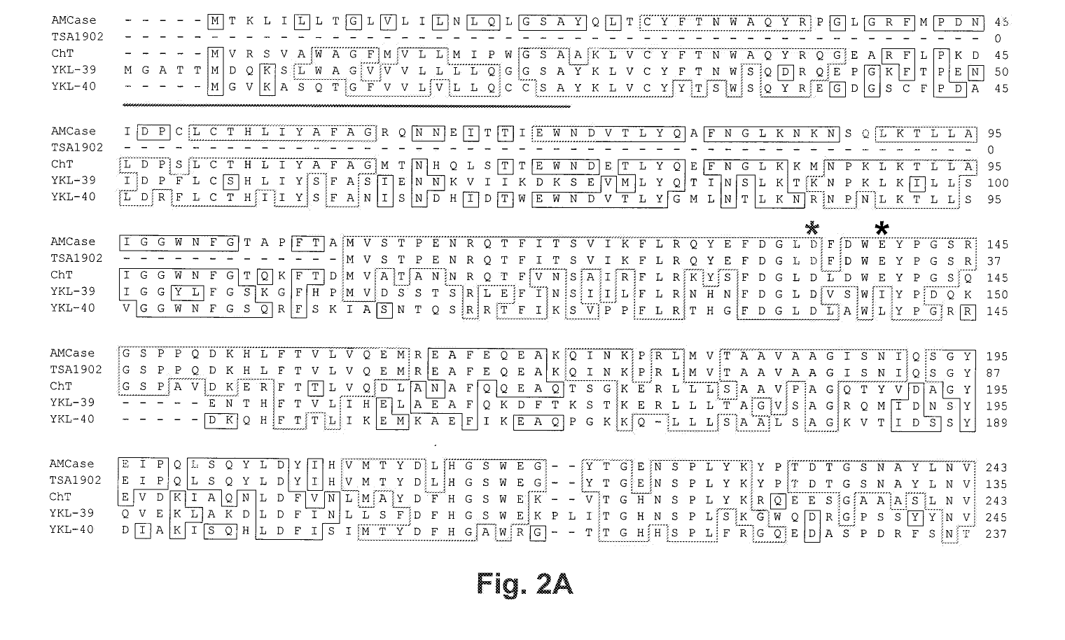

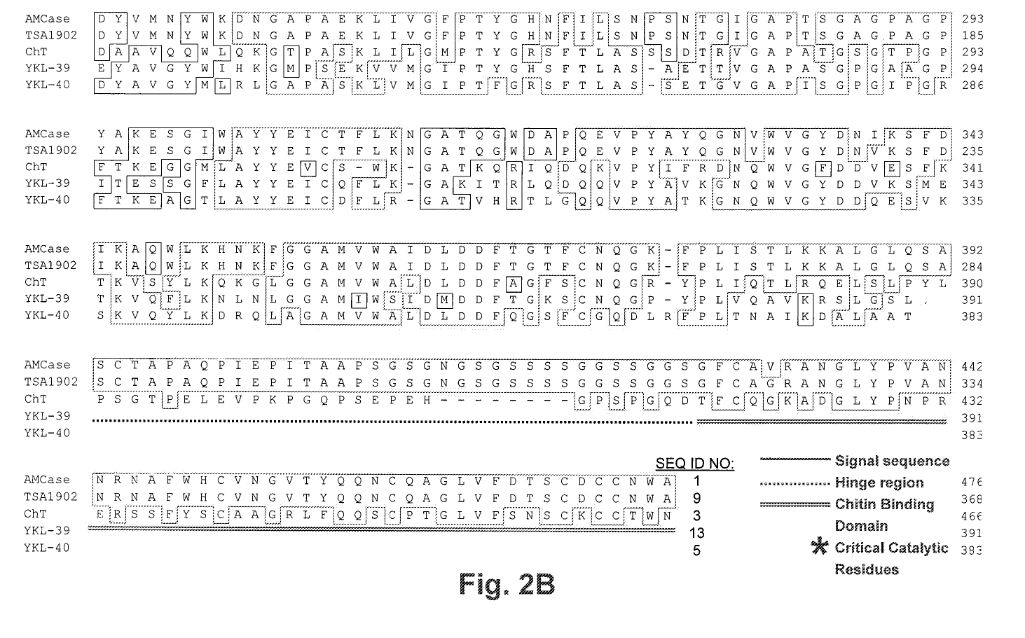

“Chitinase-like molecule” is a term recognized in the art to encompass a family of proteins that show a high degree of homology to known chitinases, but that do not demonstrate detectable chitinase activity due to one or more amino acid changes in the “catalytic” domain (see, e.g., FIGS. 1 and 2). More particularly, a chitinase-like molecule falling within the scope of the present invention will demonstrate at least about 40% identity at the amino acid level to the “catalytic” domain of at least one chitinase or chitinase-like molecule disclosed herein, and more preferably at least about 50% identity at the amino acid level. Preferred chitinase-like molecules of the present invention, among others, include oviductin, YKL-40 (in human; BRP-39 in mice), YKL-39, YM1 (also known as eosinophil chemokine ECF-L), YM2 and YM3 and any other chitinase-like molecule described herein. A more detailed description of several known chitinases and chitinase-like proteins is included below.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87: 2264-2268, modified as in Karlin and Altschul, 1993, Proc. Natl. Acad. Sci. U.S.A. 90: 5873-5877. Such an algorithm is incorporated into the NBLAST and XBLAST programs of Altschul et al., 1990, J. Mol. Biol. 215: 403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25: 3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4: 11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

Chitin/chitinase-like proteins (C/CLPs) include, but are not limited to acidic mammalian chitinase (also referred to as eosinophil chemotactic cytokine and exemplified by GenBank Acc. No. AF290003 and No. AF290004), Ym1 (also known as chitinase 3-like 3, ECF-L precursor, as exemplified by GenBank Acc. No. M94584), Ym2 as exemplified by GenBank Acc. No. AF461142, cartilage glycoprotein 1 (also referred to as BRP-39, chitinase 3-like 1, GP-39, YKL-40 and exemplified by GenBank Acc. No. X93035 and No. NM_{—}001276), chitotriosidase (also referred to as ChT, ChTRase and exemplified by GenBank Acc. No. NM_{—}003465), oviductal glycoprotein 1 (also referred to as mucin 9, oviductin and as exemplified by GenBank Acc. No. NM_{—}002557 and NP_{—}031722), cartilage glycoprotein-39 (also known as chitinase 3-like 1, GP-39, YKL 40, as exemplified by GenBank Acc. No. NM_{—}001276), HC gp-39L (as disclosed in U.S. Patent 7,067,280), chondrocyte protein 39 (also known as chitinase 3-like 2, YKL-39, as exemplified by GenBank Acc. No. NM_{—}004000), TSA1902-L as exemplified by GenBank Acc. No. AB025008, TSA1902-S as exemplified by GenBank Acc. No. AB025009; interacting chitinase-like protein (also referred to as SI-CLP gene and exemplified by GenBank Acc. No. CAF32458) and ECF-L (see, Oba et al., J Bone Miner Res. 2003 7:1332-41). Specifically included in the invention are the full-length C/CLP polypeptides, C/CLP polynucleotides which encode a C/CLP, and fragments thereof. In addition, C/CLP polynucleotides include polynucleotides sharing at least 30% identity, or at least 40% identity, or at least 50% identity, or at least 60% identity, or at least 70% identity, or at least 80% identity, or at least 90% identity, or at least 95% identity, or at least 98% identity, or at least 99% identity to a polynucleotide which encodes a C/CLP (and the complement thereof).

The skilled artisan will further appreciate that a C/CLP is a molecule that exhibits a substantial degree of homology to known chitinases (see, e.g., supra), such that it has been or can be classified as a chitinase family molecule based upon, its amino acid sequence. Further, the skilled artisan would understand, based upon the disclosure provided herein, that while a C/CLP can exhibit homology to a known chitinase, a C/CLP molecule need not demonstrate detectable chitinase activity, in that they may not detectably cleave chitin an in assay known in the art. Such C/CLPs which do not demonstrate detectable chitinase activity, include, but are not limited to, Ym1 (chitinase 3-like 3, ECF-L), Ym2, oviductal glycoprotein 1, cartilage glycoprotein 1 (BRP-39, chitinase 3-like 1, GP-39, YKL-40), oviductal glycoprotein 1 (mucin 9, oviductin), cartilage glycoprotein-39 (chitinase 3-like 1, GP-39, YKL-40), TSA1902-L, TSA1902-S and chondrocyte protein 39 (chitinase 3-like 2, YKL-39). C/CLPs are not limited to the human polypeptides, but also include the mouse orthologs, several non-limiting examples of C/CLPs with both human and mouse orthologs are listed in Table 1.

TABLE 1
Chitinase/Chitinase-Like Proteins in Mouse and Human

| Name^{a} | Human | Mouse |
|---|---|---|
| Acidic Mammalian Chitinase^{b} (AMCase, ECF-L) | + | + |
| Chitotriosidase^{b} (ChT) | + | + |
| YKL-40 (BRP39, chitinase 3-like1) | + | + |
| YKL-39 (Chondrocyte protein 39, chitinase 3-like 2) | + | |
| Oviductin (Oviductal glycoprotein 1) | + | + |
| Ym1 (ECF-L, Chitinase 3-like 3) | | + |
| Ym2 (Chitinase 3-like 4) | | + |

^{a}other common names are listed in parenthesis.
^{b}true chitinases with catalytic activity

C/CLP molecules are known in the art to have certain characteristic domains including but not limited to a signal sequence, a catalytic domain that may or may not be functional, a hinge region and a chitin binding domain (CBD). These primary domains are generally depicted in FIG. 1 and specifically identified in FIG. 2. The signal sequence is generally understood to target the protein to the secretory pathway where the protein may then be secreted or targeted to a cellular membrane. The catalytic domain in generally understood to be responsible for the catalytic activity of the enzyme, within this domain is the DXDXE motif that correspond to the putative active site in bacterial chitinases, the E residue of the chitinase DXDXE consensus is required for catalytic activity. It is noted that the Chitinase-like protein members of the C/CLP family, which do not have chitinase activity, have not maintained one or more of the critical residues in the DXDXE consensus. As reported here (see Example 1) and elsewhere that the CBD is not required for the enzyme to hydrolyze the soluble substrate however, the CBD is required for the hydrolysis of insoluble chitin (see e.g., Tjoelker et al., 2000, JBC 275:514-20; Renkema et al., 1997, J Biochem 244:279-85). It has further been demonstrated that the CBD can confer chitin binding activity when fused to an unrelated protein. The hinge region which links the CBD to the catalytic domain may facilitate secretion from the cell and in some cases may stabilized the enzyme in the presence of proteolytic enzymes but does not appear to be required for chitinase activity (Arakane et al., 2003, Insect Biochem and Mol Biol 33:631-48).

The terms “chitinase fragment”, “chitinase-like protein fragments” and “C/CLP fragments” described herein include a chitinase and/or chitinase-like protein peptide or polypeptide comprising, or alternatively consisting of (or consisting essentially of) an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of a chitinase and/or chitinase-like protein polypeptide, preferably human chitinase and/or chitinase-like protein.

The term “chitinase fragment”, “chitinase-like protein fragments” and “C/CLP fragments” described herein also specifically include polypeptides comprising, or alternatively consisting of (or consisting essentially of) an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, or at least contiguous 80 amino acid residues of a C/CLP catalytic domain (active or inactive), or a C/CLP chitin binding domain, or a C/CLP hinge region.

### 5.3 C/CLP Antagonists

A “C/CLP antagonist” according to the present invention is preferably one that modulates, decreases or inhibits at least one activity of a C/CLP. In one embodiment, a C/CLP antagonist decreases at least one activity of a C/CLP. In another embodiment, a C/CLP antagonist inhibits at least one activity of a C/CLP.

Activities of a C/CLP include, but are not limited to, chitinase activity, saccaride (e.g., GlcNAc) binding activity, galectin binding, modulation of galectin-mediated cellular responses, chemotactic activity (e.g., eosinophil chemotactic activity), simulation of inflammatory responses (e.g., those mediated by IL-13, such as the production of MCP-1, MCP-2, MIP-1β, eotaxin, eotaxin-2 and ENA-78, and those mediated directly by a C/CLP, such as production of MCP-1 and eotaxin), cell surface receptor binding, signal transduction activity. Other activities of a C/CLP include those which mediate, directly and/or indirectly, any of the variety of changes associated with inflammatory disease including, but not limited to, apoptosis, tissue inflammation, increased lung volume, increased eosinophils in bronchioalveolar ravage (BAL) fluid, increased lymphocytes in BAL fluid, increased total cells in BAL fluid, increased alveolus size, increased deposition of crystals comprising chitinase-like molecules in lung tissue, increased airway resistance, increased mucus metaplasia, increased mucin expression, increased parenchymal fibrosis, increased airway remodeling (e.g., airway thickening, mucus metaplasia, epithelial hypertrophy and airway fibrosis), increased subepithelial fibrosis, increased collagen deposition in airway tissue, epithelial hypertrophy in the lung tissue, focal organization of crystalline material into Masson body-like fibrotic foci, airway hyperresponsiveness (AHR), and the like.

In one embodiment, a C/CLP antagonist of the invention antagonizes a single C/CLP. In another embodiment, a C/CLP antagonist of the invention antagonizes multiple C/CLPs. In still another embodiment, a C/CLP antagonist of the invention antagonizes a single activity of at least one C/CLP. In yet another embodiment, a C/CLP antagonist of the invention antagonizes more then one activity of at least one C/CLP.

In one embodiment, a C/CLP antagonist of the invention, antagonizes at least one activity of a C/CLP by at least about 10% or at least about 15%, or at least about 20%, or at least about 30%, or at least about 40%, or at least about 50%, or at least about 60%, or at least about 70%, or at least about 80%, or at least about 90%, or at least about 100%, or at least about 200%, or at least about 300%, or at least about 400%, or at least about 500%. In another embodiment, a C/CLP antagonist of the invention, antagonizes at least one activity of a C/CLP by at least 10% or at least 15%, or at least 20%, or at least 30%, or at least 40%, or at least 50%, or at least 60%, or at least 70%, or at least 80%, or at least 90%, or at least 100%, or at least 200%, or at least 300%, or at least 400%, or at least 500%.

In one embodiment, a C/CLP antagonist of the invention reduces at least one activity of a C/CLP by at least about 2 fold, or at least about 5 fold, or at least about 10 fold, or at least about 20 fold, or at least about 30 fold, or at least about 40 fold, or at least about 50 fold, or at least about 60 fold, or at least about 70 fold, or at least about 80 fold, or at least about 90 fold, or at least about 100 fold, or at least about 200 fold, or at least about 500 fold, or at least about 1000 fold. In another embodiment, a C/CLP antagonist of the invention reduces at least one activity of a C/CLP by at least 2 fold, or at least 5 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold, or at least 500 fold, or at least 1000 fold.

In one embodiment, as discussed above, the invention provides C/CLP antagonists which bind and antagonize a C/CLP activity. The present invention encompasses C/CLP antagonists thereof that bind to a C/CLP selected from the group consisting of: ECF-L; acidic mammalian chitinase (AMCase); Ym1; Ym2; YKL-39; BRP-39; YKL-40; oviductal glycoprotein 1; cartilage glycoprotein 1; chitotriosidase; oviductal glycoprotein 1; cartilage glycoprotein-39; TSA1902-L, TSA1902-S and chondrocyte protein 39, wherein said C/CLP antagonist antagonizes at least one activity of the C/CLP.

It is contemplated that one or more C/CLP protein domain is involved in modulating inflammatory responses. Accordingly, a C/CLP antagonist of the invention may bind to one or more specific C/CLP protein domain or fragment thereof. In a specific embodiment, a C/CLP antagonist binds the signal sequence of a C/CLP. In another specific embodiment, a C/CLP antagonist binds the catalytic domain (functional or non-functional) of a C/CLP. In still another specific embodiment, a C/CLP antagonist binds the chitin binding domain of a C/CLP.

In one embodiment, a C/CLP antagonist of the invention binds a chitinase (e.g., AMCase). In a specific embodiment, a C/CLP antagonist binds the signal sequence of a chitinase. In another specific embodiment, a C/CLP antagonist binds the catalytic domain of a chitinase. In still another specific embodiment, a C/CLP antagonist binds the chitin binding domain of a chitinase.

In a specific embodiment, a C/CLP antagonist of the invention binds AMCase. In a specific embodiment, a C/CLP antagonist binds the signal sequence of AMCase. In another specific embodiment, a C/CLP antagonist binds the catalytic domain of AMCase. In still another specific embodiment, a C/CLP antagonist binds the chitin binding domain of AMCase.

In another embodiment a C/CLP antagonist of the invention binds a chitinase-like protein. In a specific embodiment, a C/CLP antagonist binds the signal sequence of a chitinase like protein. In another specific embodiment, a C/CLP antagonist binds the catalytic domain of a chitinase like protein. It is also contemplated that a C/CLP antagonist may bind the chitin binding domain of a chitinase-like molecule yet to be identified comprising a chitin binding domain.

The C/CLP antagonists are, in one embodiment, those that decrease or inhibit the ability of a C/CLP to mediated inflammatory responses including, but not limited to, those mediated by IL-13, such as the production of MCP-1, MCP-2, MIP-1β, eotaxin, eotaxin-2 and ENA-78, and those mediated directly by a C/CLP, such as production of TNF-α, RANTES, IL-10, IL-6, MCP-1 and eotaxin. In other embodiments, the C/CLP antagonists employed in the methods of the present invention are those that modulate one or more activity of a C/CLP including, but not limited to, chitinolytic activity, saccharide (e.g., GlcNAc) binding activity, chemotatic activity, receptor binding activity, signal transduction activity, receptor binding activity.

In one embodiment, a C/CLP antagonist, when administered to a subject in need thereof, improves (e.g., reduces the severity and/or symptoms of) one or more clinical indicator, including but not limited to, cellular infiltration of the lung (e.g., increased eosinophils in bronchioalveolar ravage (BAL) fluid, increased lymphocytes in BAL fluid, increased total cells in BAL fluid), airway function, airway inflammation, airway remodeling (e.g., airway thickening, mucus metaplasia, epithelial hypertrophy and airway fibrosis), airway hyperresponsiveness (AHR), joint inflammation, connective tissue damage, bone damage, joint mobility.

In one embodiment, a C/CLP antagonist of the invention, when administered to a subject in need thereof, improves (e.g., reduces the severity and/or symptoms of) one or more clinical indicator by at least about 10% or at least about 15%, or at least about 20%, or at least about 30%, or at least about 40%, or at least about 50%, or at least about 60%, or at least about 70%, or at least about 80%, or at least about 90%, or at least about 100%, or at least about 200%, or at least about 300%, or at least about 400%, or at least about 500%. In another embodiment, a C/CLP antagonist of the invention, antagonizes at least one activity of a C/CLP by at least 10% or at least 15%, or at least 20%, or at least 30%, or at least 40%, or at least 50%, or at least 60%, or at least 70%, or at least 80%, or at least 90%, or at least 100%, or at least 200%, or at least 300%, or at least 400%, or at least 500% as compared to an untreated control.

In one embodiment, a C/CLP antagonist of the invention, when administered to a subject in need thereof, improves (e.g., reduces the severity and/or symptoms of) one or more clinical indicator by at least about 2 fold, or at least about 5 fold, or at least about 10 fold, or at least about 20 fold, or at least about 30 fold, or at least about 40 fold, or at least about 50 fold, or at least about 60 fold, or at least about 70 fold, or at least about 80 fold, or at least about 90 fold, or at least about 100 fold, or at least about 200 fold, or at least about 500 fold, or at least about 1000 fold. In another embodiment, a C/CLP antagonist of the invention reduces at least one activity of a C/CLP by at least 2 fold, or at least 5 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold, or at least 500 fold, or at least 1000 fold as compared to an untreated control.

In one embodiment, a C/CLP antagonist, when administered to a subject in need thereof, reduces inflammation, as compared to an untreated control. In a specific embodiment, a C/CLP antagonist, when administered to a subject in need thereof, reduces cellular infiltration of the lung (e.g., reduces the number of eosinophils in bronchioalveolar ravage (BAL) fluid, reduces the number lymphocytes in BAL fluid, increased total cells in BAL fluid) as compared to an untreated control. In another specific embodiment, a C/CLP antagonist, when administered to a subject in need thereof, improves airway function as compared to an untreated control. Airway function can be measured using any of a number of methods well known in the art, commonly used pulmonary function tests (PFT) include, for example, spirometry and plethysmography. In yet another specific embodiment, a C/CLP antagonist, when administered to a subject in need thereof, reduces airway inflammation. In still another specific embodiment, a C/CLP antagonist, when administered to a subject in need thereof, reduces airway remodeling (e.g., airway thickening, mucus metaplasia, epithelial hypertrophy and airway fibrosis) as compared to an untreated control. In yet another embodiment, a C/CLP antagonist, when administered to a subject in need thereof, reduces airway hyperresponsiveness (AHR) as compared to an untreated control. Also contemplated is a C/CLP antagonist which, when administered to a subject in need thereof, reduces IL-13 mediated pulmonary disease as compared to an untreated control.

In one specific embodiment, a C/CLP antagonist, when administered to a subject in need thereof, reduces cellular infiltration of affected joints. In another specific embodiment, a C/CLP antagonist, when administered to a subject in need thereof, reduces joint inflammation as compared to an untreated control. In still another specific embodiment, a C/CLP antagonist, when administered to a subject in need thereof, reduces connective tissue damage as compared to an untreated control. In yet another specific embodiment, a C/CLP antagonist, when administered to a subject in need thereof, reduces bone damage as compared to an untreated control. In another specific embodiment, a C/CLP antagonist, when administered to a subject in need thereof, improves joint mobility as compared to an untreated control.

In one embodiment, administration of the C/CLP antagonists of the invention leads to decreased cellular infiltration associated with a disease state including but not limited to, interstitial lung disease (ILD), pulmonary fibrosis, bronchitis, chronic obstructive pulmonary disease (COPD), pneumonia, pneumonitis, acute respiratory distress syndrome (ARDS), severe acute respiratory syndrome (SARS), sarcoidosis, cystic fibrosis (CF), emphysema, asthma, smoker's cough, allergy, allergic rhinitis and sinusitis, Paget's disease, abnormal bone remodeling, osteoporosis, Gorham-Stout syndrome, osteoarthritis, rheumatoid arthritis, psoriatic arthritis and brittle bone disease.

In another embodiment, administration of the C/CLP antagonists of the invention prevents or reduced tissue damage associated with a disease state including but not limited to, interstitial lung disease (ILD), pulmonary fibrosis, bronchitis, chronic obstructive pulmonary disease (COPD), pneumonia, pneumonitis, acute respiratory distress syndrome (ARDS), severe acute respiratory syndrome (SARS), sarcoidosis, cystic fibrosis (CF), emphysema, asthma, smoker's cough, allergy, allergic rhinitis and sinusitis, Paget's disease, abnormal bone remodeling, osteoporosis, Gorham-Stout syndrome, osteoarthritis, rheumatoid arthritis, psoriatic arthritis and brittle bone disease.

In another embodiment, administration of the C/CLP antagonists of the invention leads to an improvement in the reduced airway function associated with a disease state including but not limited to, interstitial lung disease (ILD), pulmonary fibrosis, bronchitis, chronic obstructive pulmonary disease (COPD), pneumonia, pneumonitis, acute respiratory distress syndrome (ARDS), severe acute respiratory syndrome (SARS), sarcoidosis, cystic fibrosis (CF), emphysema, asthma, smoker's cough, allergy, allergic rhinitis and sinusitis.

It is contemplated that a C/CLP antagonist of the invention may be administered prophylactically to prevent or reduce the severity of one or more adverse symptom of a disease or disorder. Symptoms which may be prevented including but are not limited to, cellular infiltration of the lung, reduction airway function, airway inflammation, airway remodeling (e.g., airway thickening, mucus metaplasia, epithelial hypertrophy and airway fibrosis), airway hyperresponsiveness (AHR), joint inflammation, connective tissue damage, bone damage, decreased joint mobility. Diseases and disordered which may be treated by the prophylactic administration of a C/CLP antagonist of the invention include, but are not limited to, interstitial lung disease (ILD), pulmonary fibrosis, bronchitis, chronic obstructive pulmonary disease (COPD), pneumonia, pneumonitis, acute respiratory distress syndrome (ARDS), severe acute respiratory syndrome (SARS), sarcoidosis, cystic fibrosis (CF), emphysema, asthma, smoker's cough, allergy, allergic rhinitis and sinusitis, Paget's disease, abnormal bone remodeling, osteoporosis, Gorham-Stout syndrome, osteoarthritis, rheumatoid arthritis, psoriatic arthritis and brittle bone disease.

A C/CLP antagonist can include, but should not be construed as being limited to a chemical compound, a protein, a peptidomemetic, an antibody, a ribozymes, an siRNA, an aptamer and an antisense nucleic acid molecule. Such antagonists include, among others, those selected from the group consisting of antibodies, Fab₂ fragments, Fab fragments, Fv fragments, scFv molecules, proteins, peptides, non-peptidic agents, small molecule inhibitors, inactive fragments of C/CLPs that function as negative regulators (i.e., that bind a C/CLP receptor but do not stimulate down stream signaling pathways), and inactive chitinase-binding fragments of a C/CLP receptor (i.e., that bind chitinase but cannot signal).

In one embodiment, a C/CLP antagonist is a chemical compound. C/CLP antagonists are well known in the art, and some of the key critical elements of one class of C/CLP antagonists have been defined (Spindler and Spindler-Barth, 1999, Chitin and Chitinases, Birkhauser Verlag Basel, Switzerland). It is contemplated that C/CLP antagonists of the invention encompass already known C/CLP antagonist such as, but not limited to, allosamidin (Carbohydrate Chemistry Industrial Research Limited, Lower Hutt, New Zealand, and Eli Lilly and Co., Greenfield, Ind.) and its derivatives (see, e.g., U.S. Pat. No. 5,413,991), glucoallosamidin A, glucoallosamidin B, methyl-N demethylallosamidin (Nishimoto et al., 1991, J. Antibiotics 44:716-722) demethylallosamidin (U.S. Pat. No. 5,070,191), and didemthylallosamidin (Zhou et al., 1993, J. Antibiotics 46:1582-1588). Further contemplated C/CLP antagonists include stylogaunidine and its derivatives (Kato et al., 1995, Tetrahedron. Lett. 36:2133 2136), dipeptide cyclo-(L-Arg-D-Pro) (Izumida et al., 1996, J. Antibiotics 49:76-80), divalent cations (e.g., Cu 2+, Zn2+′ and Hg2+) (Izumida et al., 1995, J. Mar. Biotechnol. 2:163 20 166; Funke and Spindler, 1989, Comp. Biochem Physiol. 94B: 691-695), and riboflavin and; flavin derivatives (International Publication No. WO 02/23991). Additionally, a C/CLP; antagonist encompasses a chemically modified compound, and derivatives thereof. Methods for modifying and generating derivatives of a chemical compound are well known to one of skill in the chemical arts.

Further, one of skill in the art would, when equipped with this disclosure and the methods exemplified herein, appreciate that a C/CLP antagonist includes such antagonists as discovered in the future, as can be identified by well-known criteria in the art of pharmacology, such as the physiological results of inhibition of a C/CLP as described in detail herein and/or as known in the art. Therefore, the present invention is not limited in any way to any particular C/CLP antagonist as exemplified or disclosed herein; rather, the invention encompasses those C/CLP antagonists that would be understood by the routineer to be useful as are known in the art and as are discovered in the future.

Further methods of identifying and producing a C/CLP antagonist are well known to those of ordinary skill in the art, including, but not limited, obtaining an inhibitor from a naturally occurring source (i.e., Streptomyces sp., Pseudomonas sp., Stylotella aurantium). Alternatively, a C/CLP antagonist can be synthesized chemically. Further, the routineer would appreciate, based upon the teachings provided herein, that a C/CLP antagonist can be obtained from a recombinant organism. Compositions and methods for chemically synthesizing C/CLP antagonist and for obtaining them from natural sources are well known in the art and are; described in, among others, Yamada et al., U.S. Pat. Nos. 5,413,991, and 5,070,191

In another embodiment, a C/CLP antagonist is a nucleic acid molecule. In a specific embodiment a C/CLP antagonist is a nucleic acid molecule that inhibits or reduces the level of messenger RNA encoding a C/CLP including, but not limited to, an aptamer, a ribozyme, an antisense molecule, an siRNA.

In one embodiment, the invention is directed to ribozymes as antagonists of C/CLPs. As known in the art ribozymes are single stranded RNA molecules retaining catalytic activities. Their structures are based on naturally occurring site-specific, self-cleaving RNA molecules. Once they have cleaved their target, ribozymes are released from their mRNA target and are free to cleave another mRNA molecule (Usman et al., 2000, J Clin Invest 106:1197-202). It has been shown that unmodified and modified (e.g., 2′-O-methyl and phosphorothioate) synthetic ribozymes can be used to inhibit specifically gene expression in vitro (Usman et al., supra). It has been also shown that inhibition of gene expression can be achieved by administration of synthetic modified ribozymes to living organisms (Czubayko et al., 1996, PNAS USA 93:14753-8; Lee et al., 2000, Hepatolo 32:640-6; Abounader et al., 2002, Faseb J 16:108-10; Aigner et al., 2002, Gene Ther 9:1700-7).

In one embodiment, the invention is directed towards RNAi to antagonize C/CLPs. siRNA and shRNA RNA interference (RNAi), quelling or post-transcriptional gene silencing (PTGS) designate a phenomenon by which dsRNA specifically suppresses expression of a target gene at post-translational level. This mechanism, which was originally discovered in nematode worm (Caenorhabditis elegans) (Fire et al., 1998, Nature 391:806-11), has now been found in a large number of organisms including fungal Neurospora crasa (Romano et al., 1992, Mol Microbiol 6:3343-53), parasites (e.g. Tryponosomia brucei (Ngo et al., 1998, PNAS USA 95:14687-92), Planaria (Sanchez Alvarado and Newmark, 1999, PNAS USA 96:5049-54), Hydra (Lohmann et al., 1999, Dev Biol 214:211-4), Drosophila (Misquitta and Paterson, 1999, PNAS USA 96:1451-6; Zamore et al., 2000, Cell 101:25-33), zebrafish (Wargelius et al., 1999, Biochem Biophys Res Commun 263:156-61), plants (Angell et al.,1997, Embo J 16:3675-84; Ruiz et al., 1998, Plant Cell 10:937-46), and mammals (Elbashir et al., 2001, Nature 411:494-8; McManus and Sharp, 2002, Nat Rev Genet 3:737-47).

In normal conditions, RNAi is initiated by double-stranded RNA molecules (dsRNA) of several thousands of base pair length. In vivo, dsRNA introduced into a cell is cleaved into a mixture of short dsRNA molecules called short interfering RNA (siRNA). The enzyme that catalyzes the cleavage, Dicer, is an endo-RNase that contains RNase III domains (Bernstein et al., 2001, Nature 409:363-6). In mammalian cells, the siRNAs produced by Dicer are 21 23 by in length, with a 19 or 20 nucleotides duplex sequence, two-nucleotide 3′ overhangs and 5′-triphosphate extremities (Zamore et al. supra; Elbashir et al. Genes Dev 15:188-200; Elbashir et al., 2001, Embo J 20:6877 88). siRNA are usually designed against a region 50-100 nucleotides downstream the translation initiator codon, whereas 5′UTR (untranslated I region) and 3′UTR are usually avoided. Reduction of gene expression usually occurs within the 24 hours after treatment, and may persist for an extended period of time (Li et al., 2003, Cancer Res 63:3593-7). Gene inhibition by siRNA is an efficient process which is at least tenfold more potent as silencing trigger than sense or antisense RNAs alone (Fire et al. supra).

Production and delivery of antisense nucleic acids and RNAi is known in the art (e.g., as taught in PCT Publication WO 2004/016229). In one embodiment, small double-stranded interfering RNA (RNA interference (RNAi)) can be used (e.g., RNAi that targets one or more C/CLP) in the methods of the invention. RNAi is a post-transcription process, in which double-stranded RNA is introduced, and sequence-specific gene silencing results, though catalytic degradation of the targeted mRNA (see, e.g., Elbashir, S. M. et al., Nature 411:494-498 (2001); Lee, N. S., Nature Biotech. 19:500-505 (2002); and Lee, S-K. et al., Nature Medicine 8(7):681-686 (2002).

RNAi is used routinely to investigate gene function in a high throughput fashion or to modulate gene expression in human diseases (Chi et al., Proc. Natl. Acad. Sci. U.S.A., 100(11):6343-6346 (2003)). Introduction of long double stranded RNA leads to sequence-specific degradation of homologous gene transcripts. The long double stranded RNA is metabolized to small 21-23 nucleotide siRNA (small interfering RNA). The siRNA then binds to protein complex RISC (RNA-induced silencing complex) with dual function helicase. The helicase has RNase activity and is able to unwind the RNA. The unwound siRNA allows an antisense strand to bind to a target. This results in sequence dependent degradation of cognate mRNA. Aside from endogenous RNAi, exogenous RNAi, chemically synthesized or recombinantly produced RNAi can also be used in the compositions and methods of the invention.

In one embodiment, the invention is directed to aptamers of C/CLPs. As is known in the art, aptamers are macromolecules composed of nucleic acid (e.g., RNA, DNA) that bind tightly to a specific molecular target (e.g., C/CLP proteins, C/CLP domains (e.g., CBP or catalytic domains), C/CLP polypeptides and/or C/CLP epitopes as described herein). A particular aptamer may be described by a linear nucleotide sequence and an aptamer is typically about 15-60 nucleotides in length. The chain of nucleotides in an aptamer form intramolecular interactions that fold the molecule into a complex three-dimensional shape, and this three-dimensional shape allows the aptamer to bind tightly to the surface of its target molecule. Given the extraordinary diversity of molecular shapes that exist within the universe of all possible nucleotide sequences, aptamers may be obtained for a wide array of molecular targets, including proteins and small molecules. In addition to high specificity, aptamers have very high affinities for their targets (e.g., affinities in the picomolar to low nanomolar range for proteins). Aptamers are chemically stable and can be boiled or frozen without loss of activity. Because they are synthetic molecules, they are amenable to a variety of modifications, which can optimize their function for particular applications. For in vivo applications, aptamers can be modified to dramatically reduce their sensitivity to degradation by enzymes in the blood. In addition, modification of aptamers can also be used to alter their biodistribution or plasma residence time.

Selection of aptamers that can bind C/CLPs or a fragment there of (e.g., the CBD domain or a fragment thereof) can be achieved through methods known in the art. For example, aptamers can be selected using the SELEX (Systematic Evolution of Ligands by Exponential Enrichment) method (Tuerk, C., and Gold, L., Science 249:505-510 (1990)). In the SELEX method, a large library of nucleic acid molecules (e.g., 10¹⁵ different molecules) is produced and/or screened with the target molecule (e.g., C/CLP proteins, C/CLP domain (e.g., CBP or catalytic domain), C/CLP polypeptides and/or C/CLP epitopes as described herein). The target molecule is allowed to incubate with the library of nucleotide sequences for a period of time. Several methods can then be used to physically isolate the aptamer target molecules from the unbound molecules in the mixture and the unbound molecules can be discarded. The aptamers with the highest affinity for the target molecule can then be purified away from the target molecule and amplified enzymatically to produce a new library of molecules that is substantially enriched for aptamers that can bind the target molecule. The enriched library can then be used to initiate a new cycle of selection, partitioning, and amplification. After 5-15 cycles of this selection, partitioning and amplification process, the library is reduced to a small number of aptamers that bind tightly to the target molecule. Individual molecules in the mixture can then be isolated, their nucleotide sequences determined, and their properties with respect to binding affinity and specificity measured and compared. Isolated aptamers can then be further refined to eliminate any nucleotides that do not contribute to target binding and/or aptamer structure (i.e., aptamers truncated to their core binding domain). See Jayasena, S. D. Clin. Chem. 45:1628-1650 (1999) for review of aptamer technology.

In particular embodiments, the C/CLP antagonists of the invention are aptamers that have the binding specificity and/or functional activity described herein for the antibodies of the invention (see infra). Thus, for example, in certain embodiments, the present invention is drawn to aptamers that have the same or similar binding specificity as described herein for the antibodies of the invention (see, infra) (e.g., binding specificity for a mammalian C/CLP polypeptide, fragments of mammalian C/CLP polypeptides (e.g., C/CLP special domains), epitopic regions of mammalian C/CLP polypeptides (e.g., epitopic regions of C/CLP that are bound by the antibodies of the invention). In particular embodiments, the aptamers of the invention can bind to a C/CLP polypeptide and inhibit one or more functions of the C/CLP polypeptide as described herein.

In one embodiment, a C/CLP antagonist of the invention is a polypeptide molecule (including, but not limited to, proteins, post-translationally modified proteins, antibodies etc.). In a specific embodiment, a C/CLP antagonist of the invention is a polypeptide comprising a C/CLP binding portion of a galectin. In certain embodiments, a polypeptide comprising a C/CLP binding portion of a galectin does not mediate galectin cellular responses (e.g., cell-cell adhesion, apoptosis, programmed cell death, cellular activation and mitosis). In one embodiment, the C/CLP binding portion of a galectin is derived from a galectin selected from the group consisting of galectin-1, galectin-2, galectin-3, galectin-4, galectin-5, galectin-6, galectin-7, galectin-8, galectin-9, galectin-10, galectin-11, galectin-12, galectin-13 and galectin-14. In a specific embodiment, the C/CLP binding portion of a galectin is derived from galectin-3 and/or galectin-4. In certain embodiments, the C/CLP binding portion of a galectin is fused to a heterologous polypeptide. For example, the C/CLP binding portion of a galectin may be fused to an antibody Fc domain, an albumin polypeptide. Other domains useful for the generation of fusion proteins are well known in the art.

In another specific embodiment, a C/CLP antagonist of the invention is an antibody. In another specific embodiment, antibody C/CLP antagonists specifically bind a C/CLP of the invention, for example, AMCase. Also contemplated are antibody C/CLP antagonists that bind more then one C/CLP. Antibody antagonists of C/CLPs are referred to herein as a “C/CLP antibodies of the invention,” “C/CLP antibodies,” and like terms. In one specific embodiment, antibody C/CLP antagonists specifically bind human AMCase. In another specific embodiment, antibody C/CLP antagonists specifically bind human YKL-40. In still another specific embodiment, antibody C/CLP antagonists specifically bind a human TSA1902 polypeptide. In still another specific embodiment, antibody C/CLP antagonists specifically bind human chitotrosidase (ChT). In certain embodiments, antibody C/CLP antagonists specifically bind at least one human C/CLP selected from the group including but not limited to, AMCase, ChT, TSA1902 and YLK-40.

C/CLP antibodies of the invention include, but are not limited to, monoclonal antibodies, multispecific antibodies, human antibodies, humanized antibodies, camelised antibodies, chimeric antibodies, single-chain Fvs (scFv), disulfide-linked Fvs (sdFv), Fab fragments, F (ab′) fragments, and anti-idiotypic (anti-Id) antibodies (including, e.g., anti-Id antibodies to antibodies of the invention), and epitope-binding fragments of any of the above. In particular, antibodies include immunoglobulin molecules and immunologically active fragments of immunoglobulin molecules, i.e., molecules that contain an antigen binding site, these fragments may or may not be fused to another immunoglobulin domain including but not limited to, an Fc region or fragment thereof As outlined herein, the terms “antibody” and “antibodies” specifically include the antibodies described herein, full length antibodies and Fc-Fusions comprising Fc regions, or fragments thereof, fused to an immunologically active fragment of an immunoglobulin (e.g., a fragment that specifically binds a C/CLP) or to other proteins as described herein. Such Fc fusions include but are not limited to, scFv-Fc fusions, variable region (e.g., VL and VH) -Fc fusions, scFv-scFv-Fc fusions. Immunoglobulin molecules can be of any type (e.g., IgG, IgE, IgM, IgD, IgA and IgY), class (e.g., IgG1, IgG2, IgG3, IgG4, IgA1 and IgA2) or subclass.

As used herein, the term “specifically binds to a C/CLP” and analogous terms refer to peptides, polypeptides, proteins, fusion proteins and antibodies or fragments thereof that specifically bind to a C/CLP of the invention or a fragment thereof C/CLPs to which a peptide, polypeptide, protein, or antibody may specifically bind to include, but are not limited to, AMCase, Ym1, Ym2, BRP-39, YKL-40, chitotriosidase, oviductin, YKL-39, TSA1902-L, TSA1902-S, ECF-L, and others described herein. A peptide, polypeptide, protein, or antibody that specifically binds to a C/CLP of the invention or a fragment thereof may bind to other peptides, polypeptides, or proteins with lower affinity as determined by, e.g., immunoassays, BIAcore, or other assays known in the art. Antibodies or fragments that specifically bind to a C/CLP or a fragment thereof may be cross-reactive with related antigens. For example, a peptide, polypeptide, protein, or antibody that specifically binds to the CLP,Ym1, may be cross-reactive to the highly related CLP, Ym2. It is contemplated that antibodies or fragments that specifically bind to a C/CLP or a fragment thereof preferentially bind said C/CLP over other antigens. However, the present invention specifically encompasses antibodies with multiple specificities (e.g., an antibody with specificity for two or more discrete antigens (reviewed in Cao et al., 2003, Adv Drug Deliv Rev 55:171-197; Hudson et al., 2003, Nat Med 1:129-134)). For example, bispecific antibodies contain two different binding specificities fused together. In the simplest case a bispecific antibody would bind to two adjacent epitopes on a single target antigen, such an antibody would not cross-react with other antigens (as described supra). Alternatively, bispecific antibodies can bind to two different antigens. Such an antibody specifically binds to two different molecules, but not to other unrelated molecules. In addition, an antibody that specifically binds a C/CLP may cross-react with related C/CLPs. Another class of multispecific antibodies may recognize a shared subunit of multi-subunit complexes in the context of one or more specific complexes. It is also contemplated that a C/CLP antibody of the invention may bind to a common epitope shared by more then one C/CLP.

Antibodies or fragments that specifically bind to a C/CLP or a fragment thereof can be identified, for example, by immunoassays, BIAcore, or other techniques known to those of skill in the art. An antibody or fragment thereof binds specifically to a C/CLP or a fragment thereof when it binds to a C/CLP or a fragment thereof with higher affinity than to any cross-reactive antigen as determined using experimental techniques, such as radioimmunoassays (RIA) and enzyme-linked immunosorbent assays (ELISAs). See, e.g., Paul, ed., 1989, Fundamental Immunology Second Edition, Raven Press, New York at pages 332-336 for a discussion regarding antibody specificity.

Other molecules specifically contemplated as C/CLP antagonists are small, engineered protein domains such as, for example, immuno-domains and/or monomer domains (see for example, U.S. Patent Publication Nos. 2003082630 and 2003157561. Immuno-domains contain at least one complementarity determining region (CDR) of an antibody while monomer domains are based upon known naturally-occurring, non-antibody domain families, specifically protein extracellular domains, which contain conserved scaffold and variable binding sites, an example is the LDL receptor A domain which is involved in ligand binding. Such protein domains can correctly fold independently or with limited assistance from, for example, a chaperonin or the presence of a metal ion. This ability avoids mis-folding of the domain when it is inserted into a new protein environment, thereby preserving the protein domain's binding affinity for a particular target. The variable binding sites of the protein domains are randomized using various diversity generation methods such as, for example, random mutagenesis, site-specific mutagenesis, as well as by directed evolution methods, such as , for example, recursive error-prone PCR, recursive recombination and the like. For details of various diversity generation methods see U.S. Pat. Nos.5,811,238; 5,830,721; 5,834,252; PCT Publication Nos. WO 95/22625; WO 96/33207; WO 97/20078; WO 97/35966; WO 99/41368; WO 99/23107; WO 00/00632; WO 00/42561; and WO 01/23401. The mutagenized protein domains are then expressed using a display system such as, for example, phage display, which can generate a library of at least 10¹⁰ variants and facilitate isolation of those protein domains with improved affinity and potency for an intended target by subsequent panning and screening. Such methods are described in PCT publication Nos. WO 91/17271; WO 91/18980; WO 91/19818; WO 93/08278. Examples of additional display systems are described in U.S. Pat. Nos. 6,281,344; 6,194,550; 6,207,446; 6,214,553 and 6,258,558. Utilizing these methods a high diversity of engineered protein domains having sub-nM binding affinity (Kd) and blocking function (IC50) can be rapidly generated.

Once identified two to ten such engineered protein domains can be linked together, using natural protein linkers of about 4-15 amino acids in length, to form a binding protein. The individual domains can target a single type of protein or several, depending upon the use/disease indication. The engineered protein domains have several advantages over other types of polypeptide molecules including small size (average 4.5 kD per domain) which allows for good tissue penetration, high stability due to the requirement for disulfide bonds and low immunogenicity. In addition, the engineered protein domains are non-glycosylated allowing for high yield production via bacterial expression systems and are protease resistant. Finally, the serum half-life can readily be extended by binding the designing one domain to bind human serum IgG, human serum albumin or another prevalent target while the remaining domains bind to one or more desired therapeutic target.

### 5.4 C/CLP Antibodies of the Invention

As discussed above, the invention provides antibodies or fragments thereof which specifically bind and antagonize a C/CLP activity. The present invention encompasses C/CLP antibodies or fragments thereof that specifically bind to a C/CLP selected from the group consisting of: ECF-L; acidic mammalian chitinase (AMCase); Ym1; Ym2; YKL-39; BRP-39; YKL-40; HC gp-39L; oviductal glycoprotein 1; cartilage glycoprotein 1; chitotriosidase; oviductal glycoprotein 1; cartilage glycoprotein-39; TSA1902-L, TSA1902-S and chondrocyte protein 39, wherein said antibody or fragment thereof antagonizes at least one activity of the C/CLP. In one embodiment, a C/CLP antibody inhibits the interaction of a C/CLP and a galectin. In another embodiment, a C/CLP antibody inhibits the chitinase activity of a C/CLP. In a specific embodiment, a C/CLP antibody inhibits the ability of a C/CLP to mediate inflammatory responses including, but not limited to, those mediated by IL-13, such as the production of MCP-1, MCP-2, MIP-1β, eotaxin, eotaxin-2 and ENA-78, and those mediated directly by a C/CLP, such as production of TNF-α, RANTES, IL-10, IL-6, MCP-1 and eotaxin. In another specific embodiment, a C/CLP antibody, when administered to a subject in need thereof, improves (e.g., reduces the severity and/or symptoms of) one or more clinical indicator, including but not limited to, cellular infiltration of the lung (e.g., increased eosinophils in bronchioalveolar ravage (BAL) fluid, increased lymphocytes in BAL fluid, increased total cells in BAL fluid), airway function, airway inflammation, airway remodeling (e.g., airway thickening, mucus metaplasia, epithelial hypertrophy and airway fibrosis), airway hyperresponsiveness (AHR), joint inflammation, connective tissue damage, bone damage, joint mobility. Additional, C/CLP activities which may be inhibited by a C/CLP antibody of the invention have been described above. It will be understood by one of skill in the art that a C/CLP antibody may antagonize at least one activity of a C/CLP in vivo and/or in vitro.

In certain embodiments, a C/CLP antibody specifically binds the signal sequence of a C/CLP. In yet another specific embodiment, a C/CLP antibody specifically binds the catalytic domain of a C/CLP. In still another specific embodiment, a C/CLP antibody specifically binds the chitin binding domain of a C/CLP.

In one embodiment, a C/CLP antibody of the invention specifically binds a chitinase (e.g., AMCase). In a specific embodiment, a C/CLP antibody specifically binds the signal sequence of a chitinase. In another specific embodiment, a C/CLP antibody specifically binds the catalytic domain of a chitinase. In still another specific embodiment, a C/CLP antibody specifically binds the chitin binding domain of a chitinase.

In another embodiment a C/CLP antibody of the invention specifically binds a chitinase-like protein. In a specific embodiment, a C/CLP antibody specifically binds the signal sequence of a chitinase like protein. In another specific embodiment, a C/CLP antibody specifically binds the catalytic domain of a chitinase like protein. It is also contemplated that a C/CLP antibody specifically binds the chitin binding domain of a chitinase-like molecule yet to be identified comprising a chitin binding domain.

The present invention further encompasses C/CLP antibodies of the invention that have a high binding affinity for a C/CLP of the invention. In a specific embodiment, a C/CLP antibody of the invention specifically binds to a C/CLP with an association rate constant or kₒₙ, rate of at least 1×10⁵ M⁻¹s⁻¹, at least 5×10⁵M⁻¹s⁻¹, at least 1×10⁶ M⁻¹s⁻¹, at least 5×10⁶ M⁻¹s⁻¹, at least 1×10⁷ M⁻¹s⁻¹, at least 5×10⁷ M⁻¹s⁻¹, or at least 1×10⁸ M⁻¹s⁻¹. In a further specific embodiment, a C/CLP antibody of the invention specifically binds to a C/CLP with an association rate constant or kₒₙ rate of at least about 1×10⁵ M⁻¹s⁻¹, at least about 5×10⁵ M⁻¹s⁻¹, at least about 1×10⁶ M⁻¹s⁻¹, at least about 5×10⁶ M⁻¹s⁻¹, at least about 1×10⁷ M⁻¹s⁻¹, at least about 5×10⁷ M⁻¹s⁻¹, or at least about 1×10⁸ M⁻¹s⁻¹. In another embodiment, a C/CLP antibody specifically binds to a C/CLP with a kₒₙ of at least 2×10⁵ M⁻¹s⁻¹, at least 5×10⁵ M⁻¹s⁻¹, at least 1×10⁶ M⁻¹s⁻¹, at least 5×10⁶ M⁻¹s⁻¹, at least 1×10⁷ M⁻¹s⁻¹, at least 5×10⁷ M⁻¹s⁻¹, or at least 1×10⁸ M⁻¹s⁻¹. In a further embodiment, a C/CLP antibody specifically binds to a C/CLP with a kₒₙ of at least about 2×10⁵ M⁻¹s⁻¹, at least about 5×10⁵ M⁻¹s⁻¹, at least about 1×10⁶ M⁻¹s⁻¹, at least about 5×10⁶ M⁻¹s⁻¹, at least about 1×10⁷ M⁻¹s⁻¹, at least about 5×10⁷ M⁻¹s⁻¹, or at least about 1×10⁸ M⁻¹s⁻¹.

In another embodiment, a C/CLP antibody of the invention specifically binds to a C/CLP of the invention with a k_{off} rate of less than 1×10⁻¹ s⁻¹, less than 5×10⁻¹ s⁻¹, less than 1×10⁻² s⁻¹, less than 5×10⁻² s⁻¹, less than 1×10⁻³ s⁻¹, less than 5×10⁻³ s⁻¹, less than 1×10⁴ s¹, less than 5×10⁴ s¹, less than 1×10⁵ s¹, less than 5×10⁵ s¹, less than 1×10⁶ s¹, less than 5×10⁻⁶ s⁻¹, less than 1×10⁻⁷ s⁻¹, less than 5×10⁻⁷ s⁻¹, less than 1×10⁻⁸ s⁻¹, less than 5×10⁻⁸ s⁻¹, less than 1×10⁻⁹ s⁻¹, less than 5×10⁻⁹ s⁻¹, or less than 1×10⁻¹⁰⁻¹ s⁻¹. In still another embodiment, a C/CLP antibody of the invention specifically binds to aC/CLP with a k_{off} rate of less than about 1×10⁻¹ s⁻¹, less than about 5×10⁻¹ s⁻¹, less than about 1×10⁻² s⁻¹, less than about 5×10⁻² s⁻¹, less than about 1×10⁻³ s⁻¹, less than about 5×10⁻³ s⁻¹, less than about 1×10⁻⁴ s⁻¹, less than about 5×10⁻⁴ s⁻¹, less than about 1×10⁻⁵ s⁻¹, less than about 5×10⁻⁵ s⁻¹, less than about 1×10⁻⁶ s⁻¹, less than about 5×10⁻⁶ s⁻¹, less than about 1×10⁻⁷ s⁻¹, less than about 5×10⁻⁷ s⁻¹, less than about 1×10⁻⁸ s⁻¹, less than about 5×10⁻⁸ s⁻¹, less than about 1×10⁻⁹ s⁻¹, less than about 5×10⁻⁹ s⁻¹, or less than about 1×10⁻¹⁰⁻¹ s⁻¹. In a further embodiment, a C/CLP antibody specifically binds to a C/CLP with a k_{off}, of less than 5×10⁻⁴ s⁻¹, less than 1×10⁻⁵ s⁻¹, less than 5×10⁻⁵ s⁻¹, less than 1×10⁻⁶ s⁻¹, less than 5×10⁻⁶ s⁻¹, less than 1×10⁻⁷ s⁻¹, less than 5×10⁻⁷ s⁻¹, less than 1×10⁻⁸ s⁻¹, less than 5×10⁻⁸ s⁻¹, less than 1×10⁻⁹ s⁻¹, less than 5×10⁻⁹ s⁻¹, or less than 1×10⁻¹⁰ s⁻¹. In another embodiment, a C/CLP antibody specifically binds to a C/CLP with a k_{off}, of less than about 5×10⁻⁴ s⁻¹, less than about 1×10⁻⁵ s⁻¹, less than about 5×10⁻⁵ s⁻¹, less than about 1×10⁻⁶ s⁻¹, less than about 5×10⁻⁶ s⁻¹, less than about 1×10⁻⁷ s⁻¹, less than about 5×10⁻⁷ s⁻¹, less than about 1×10⁻⁸ s⁻¹, less than about 5×10⁻⁸ s⁻¹, less than about 1×10⁹ s¹, less than about 5×10⁹ s¹, or less than about 1×10¹⁰ s¹.

In another embodiment, a C/CLP antibody of the invention specifically binds to a C/CLP of the invention with an affinity constant or Kₐ(kₒₙ/k_{off}) of at least 1×10²M⁻¹, at least 5×10²M⁻¹, at least 1×10³M⁻¹, at least 5×10³M^{31 1}, at least 1×10⁴M⁻¹, at least 5×10⁴M⁻¹, at least 1×10⁵M⁻¹, at least 5×10⁵M⁻¹, at least 1×10⁶M¹, at least 5×10⁶M⁻¹, at least 1×10⁷M⁻¹, at least 5×10⁷M⁻¹, at least 1×10⁸M⁻¹, at least 5×10⁸M⁻¹, at least 1×10⁹M⁻¹, at least 5×10⁹M⁻¹, at least 1×10¹⁰M⁻¹, at least 5×10¹M⁻¹, at least 1×10¹¹M⁻¹, at least 5×10¹¹M⁻¹, at least 1×10¹²M⁻¹, at least 5×10¹²M, at least 1×10¹³M⁻¹, at least 5×10¹³M⁻¹, at least 1×10¹⁴M⁻¹, at least 5×10¹⁴M⁻¹, at least 1×10¹⁵M⁻¹, or at least 5×10¹⁵M⁻¹. In a further embodiment, a C/CLP antibody of the invention specifically binds to a C/CLP with an affinity constant or Kₐ(kₒₙ/k_{off}) of at least about 1×10²M⁻¹, at least about 5×10²M⁻¹, at least about 1×10³M⁻¹, at least about 5×10³M⁻¹, at least about 1×10⁴M⁻¹, at least about 5×10⁴M⁻¹, at least about 1×10⁵M⁻¹, at least about 5×10⁵M⁻¹, at least about 1×10⁶M⁻¹, at least about 5×10⁶M⁻¹, at least about 1×10⁷M⁻¹, at least about 5×10⁷M¹, at least about 1×10⁸M¹, at least about 5×10⁸M¹, at least about 1×10⁹M⁻¹, at least about 5×10⁹M⁻¹, at least about 1×10¹⁰M⁻¹, at least about 5×10¹M⁻¹, at least about 1×10¹¹M⁻¹, at least about 5×10¹¹M⁻¹, at least about 1×10¹²M⁻¹, at least about 5×10¹²M, at least about 1×10¹³M⁻¹, at least about 5×10¹³M⁻¹, at least about 1×10¹⁴M⁻¹, at least about 5×10¹⁴M⁻¹, least about 1×10¹⁵M⁻¹, or at least about 5×10¹⁵M⁻¹.

In yet another embodiment, a C/CLP antibody specifically binds to a C/CLP of the invention with a dissociation constant or K_{d}(k_{off}/kₒₙ) of less than 1×10⁻²M, less than 5×10⁻²M, less than 1×10⁻³M, less than 5×10⁻³M, less than 1×10⁻⁴M, less than 5×10⁻⁴M, less than 1×10⁻⁵M, less than 5×10⁻⁵M, less than 1×10⁻⁶M, less than 5×10⁻⁶M, less than 1×10⁻⁷M, less than 5×10⁻⁷M, less than 1×10⁻⁸M, less than 5×10⁻⁸M, less than 1×10⁻⁹M, less than 5×10⁻⁹M, less than 1×10⁻¹⁰M, less than 5×10⁻¹⁰M, less than 1×10⁻¹¹M, less than 5×10⁻¹¹M, less than 1×10⁻¹²M, less than 5×10⁻¹²M, less than 1×10⁻¹³M, less than 5×10⁻¹³M, less than 1×10⁻¹⁴M, less than 5×10⁻¹⁴M, less than 1×10⁻¹⁵M, or less than 5×10⁻¹⁵M. In a further embodiment, a C/CLP antibody specifically binds to a C/CLP with a dissociation constant or K_{d}(k_{off}/kₒₙ) of less than about 1×10⁻²M, less than about 5×10⁻²M, less than about 1×10⁻³M, less than about 5×10⁻³M, less than about 1×10⁻⁴M, less than about 5×10⁻⁴M, less than about 1×10⁻⁵M, less than about 5×10⁻⁵M, less than about 1×10⁻⁶M, less than about 5×10⁻⁶M, less than about 1×10⁻⁷M, less than about 5×10⁻⁷M, less than about 1×10⁻⁸M, less than about 5×10⁻⁸M, less than about 1×10⁻⁹M, less than about 5×10⁻⁹M, less than about 1×10⁻¹⁰M, less than about 5×10⁻¹⁰M, less than about 1×10⁻¹¹M, less than about 5×10⁻¹¹M, less than about 1×10¹²M, less than about 5×10¹²M, less than about 1×10¹³M, less than about 5×10¹³M, less than about 1×10⁻¹⁴M, less than about 5×10⁻¹⁴M, less than about 1×10⁻¹⁵M, or less than about 5×10⁻¹⁵M.

In certain embodiments, a C/CLP antibody specifically binds to a C/CLP of the invention with a minimal titer of at least 100K, or at least 200K, or at least 300K, or at least 400 K, or at least 500 K, or at least 600 K, or at least 700 K, or at least 800 K, or at least 1 M, or at least 1.2 M, or at least 1.4 M, or at least 1.6 M, or at least 1.8 M, or at least 2.0 M, or at least 2.2 M, or at least 2.4 M, or at least 2.6 M, or at least 2.8 M, or at least 3.0 M. In further embodiments, a C/CLP antibody specifically binds to a C/CLP of the invention with a minimal titer of at least about 100K, or at least about 200K, or at least about 300K, or at least about 400 K, or at least about 500 K, or at least about 600 K, or at least about 700 K, or at least about 800 K, or at least about 1 M, or at least about 1.2 M, or at least about 1.4 M, or at least about 1.6 M, or at least about 1.8 M, or at least about 2.0 M, or at least about 2.2 M, or at least about 2.4 M, or at least about 2.6 M, or at least 2.8 about M, or at least 3.0 about M. As used herein the term “minimal titer” refers to the maximum dilution of an antibody in solution (e.g., polyclonal rabbit serum or hybridoma cell supernatant) for which a signal 2 times background is seen by ELISA. The abbreviations “K” and “M” refer to a dilution factor of 1,000 and 1,000,000, respectively (i.e., 400 K is equivalent to 400,000).

C/CLP antibodies of the present invention may also be described or specified in terms of their cross-reactivity. Antibodies that do not bind any other analog, ortholog, or homolog of the C/CLP to which they specifically bind are included. Antibodies specific for a particular C/CLP that also bind other polypeptides (and polypeptide fragments) with at least 95%, at least 90%, at least 85%, at least 80%, at least 75%, at least 70%, at least 65%, at least 60%, at least 55%, and at least 50% identity (as calculated using methods known in the art and described herein) to said C/CLP polypeptide (e.g., a human AMCase, a human YKL-40) are also included in the present invention. In specific embodiments, the C/CLP antibodies of the present invention bind at least two C/CLP proteins. In a specific embodiment, antibodies of the present invention bind to AMCase and ChT. In another specific embodiment, antibodies of the present invention bind to AMCase and YLK-40. In another specific embodiment, antibodies of the present invention bind to AMCase and YKL-39. In another specific embodiment, antibodies of the present invention bind to ChT and YKL-40. In another specific embodiment, antibodies of the present invention bind to ChT and YKL-39. In another specific embodiment, antibodies of the present invention bind to YKL-40 and YKL-39. In another specific embodiment, antibodies of the present invention bind to AMCase, ChT and YKL-40. In another specific embodiment, antibodies of the present invention bind to AMCase, ChT and YLK-39. In another specific embodiment, antibodies of the present invention bind to ChT, YKL-40 and YLK-39. In certain embodiments, antibodies of the present invention cross-react with murine, rat and/or rabbit homologs of human C/CLP proteins and the corresponding epitopes thereof.

Antibodies that bind a particular C/CLP polypeptide of the invention, but do not bind homologous polypeptides with less than 95%, less than 90%, less than 85%, less than 80%, less than 75%, less than 70%, less than 65%, less than 60%, less than 55%, and less than 50% identity (as calculated using methods known in the art and described herein) to said C/CLP polypeptide of the present invention are also included in the present invention.

In one embodiment, the C/CLP antibodies of the invention are chimeric antibodies. In a specific embodiment, the C/CLP antibodies of the invention or fragments thereof are human or humanized antibodies.

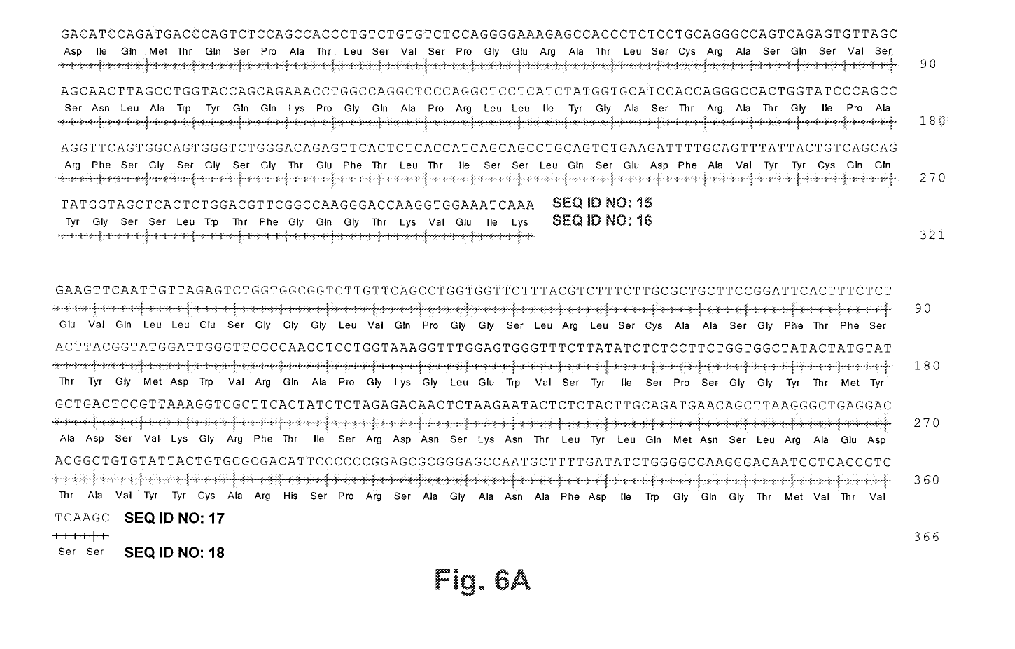

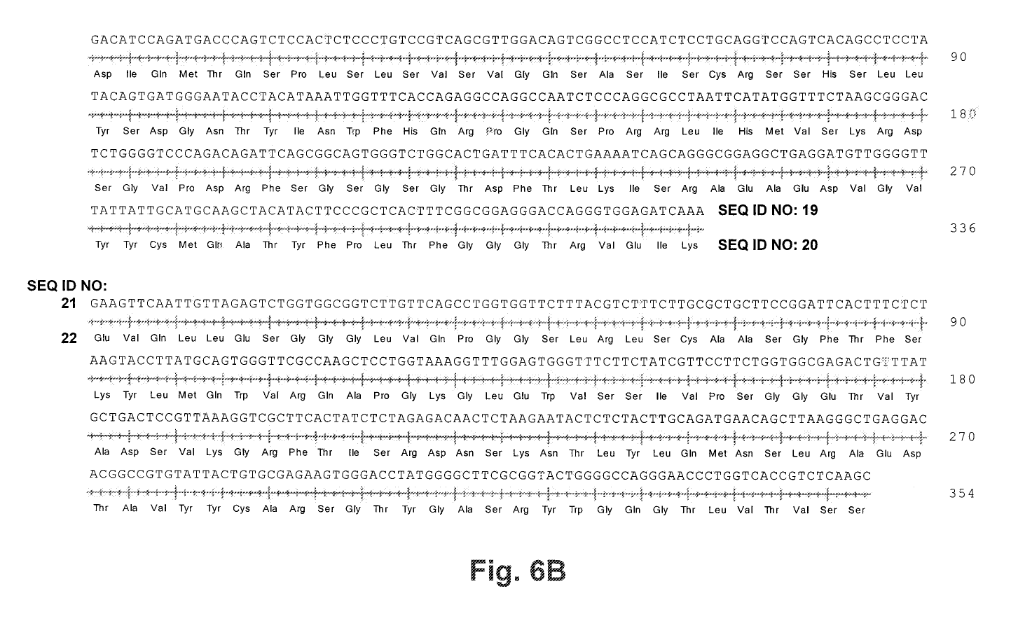

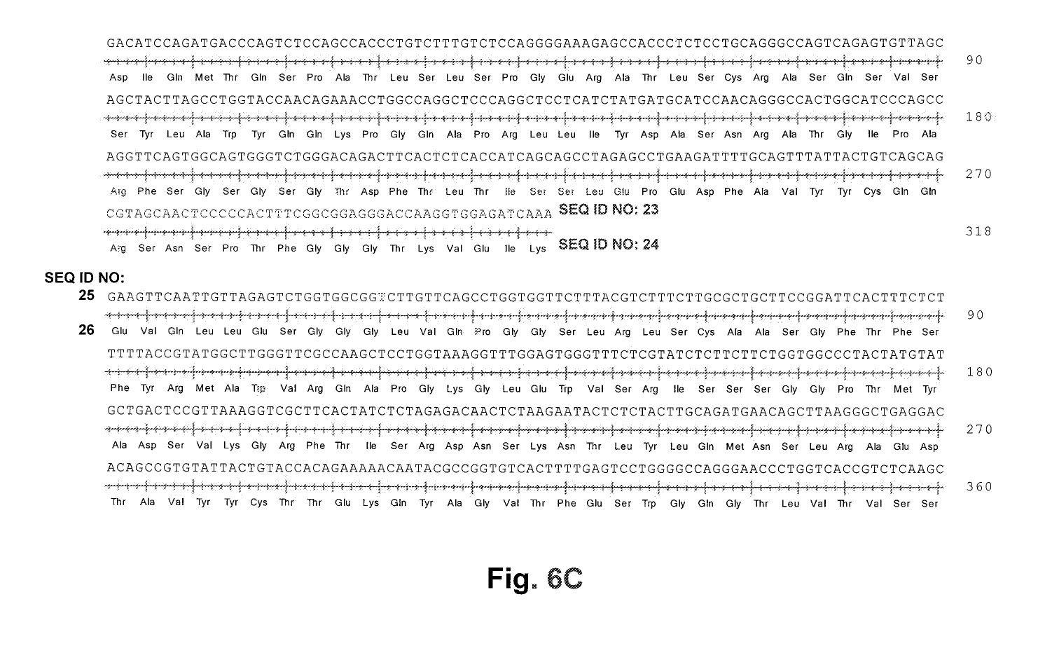

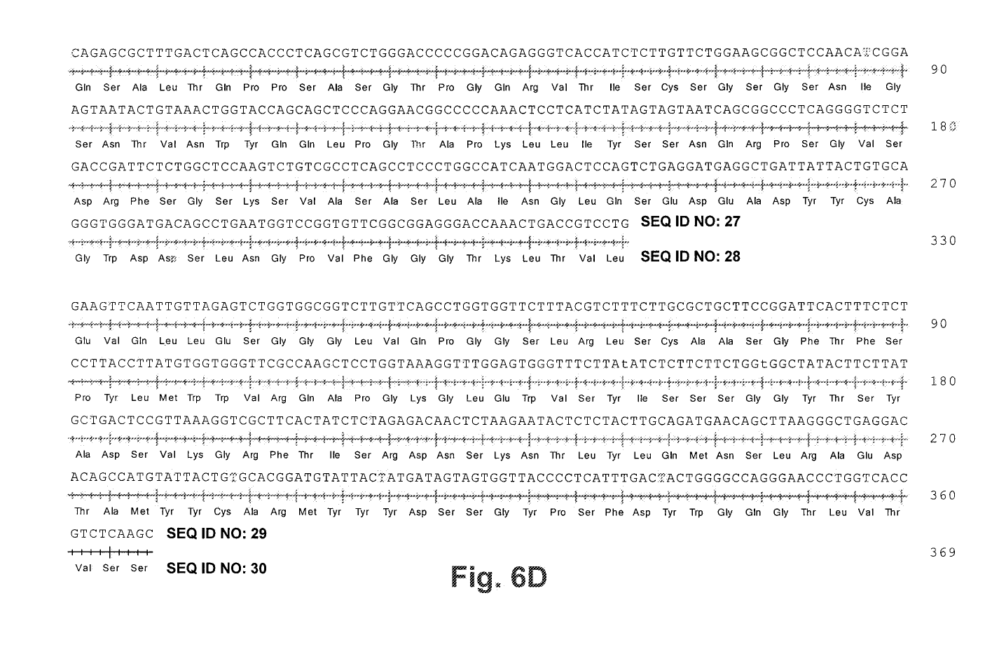

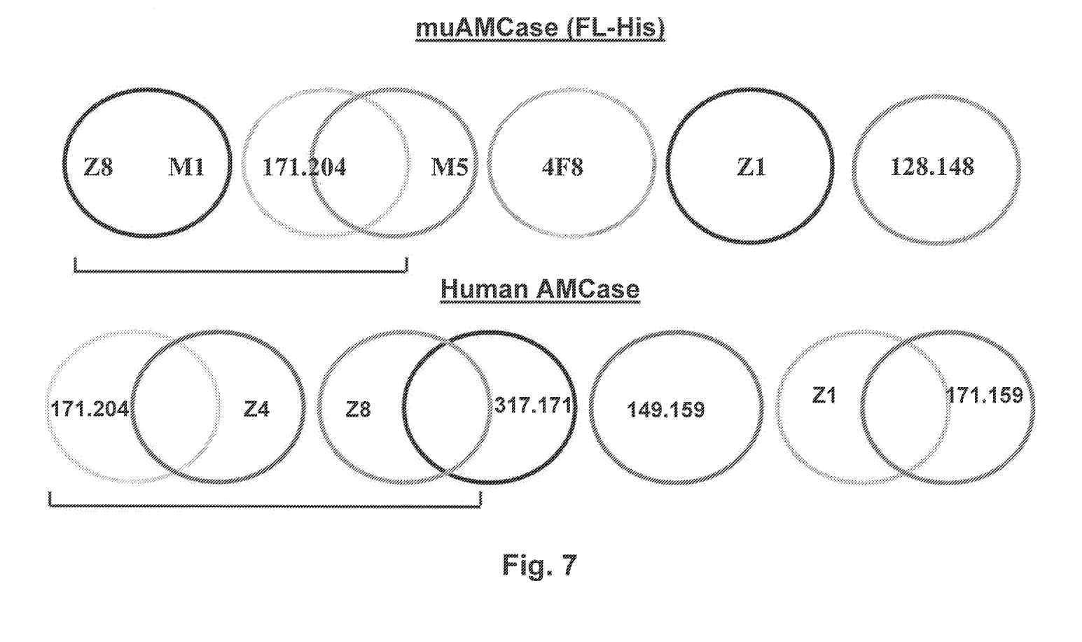

In one embodiment, the C/CLP antibodies of the invention include particular antibodies (and fragments and derivatives thereof) that specifically bind a C/CLP. In particular are the antibodies referred to herein as “AMC182-2S.171.204” which is abbreviated herein as “171.204”; “AMC182-2S.317.171” which is abbreviated herein as “317.171”; “AMC183-2.149.159” which is abbreviated herein as “149.159”; “AMC183-2.171.159” which is abbreviated herein as “171.159”; “4F8.C3.B6.G12” which is abbreviated herein as “4F8”; “AMC183-2.128.148” which is abbreviated herein as “128.148”; “Z1”; “Z4”; “Z8”; “M1”; “M5”; “1903”; “102”; and “204”. Antibodies having at least one, at least two, at least three, at least four, at least five, or all six of the CDRs of these antibodies are specific embodiments of the invention. Isolated polynucleotides that encode these antibodies (and fragments thereof) are also embodiments of the invention. Antibodies that bind to the same epitopes as these antibodies are also embodiments of the invention, as are antibodies that compete for binding with any of the above listed antibodies. The binding characteristics for “171.204,” “317.171,” “149.159,” “171.159,” “4F8,” “128.148,” “Z1,” “Z4,” “Z8,” “M1,” and “M5,” are listed in Table 2 and further detailed in FIG. 7. The nucleotide and amino acid sequences of the variable domains of the human anti-AMCase antibodies M1, M5, Z1 and Z8 are shown in FIG. 6, panels A to D, respectively.

The hybridoma cell line producing 171.240 has been deposited with the American Type Culture Collection (10801 University Boulevard, Manassas, Va. 20110-2209) and assigned ATCC Deposit No. PTA-7131 (Deposited Oct. 5, 2054). This deposit will be maintained under the terms of the Budapest Treaty on the International Recognition of the Deposit of Microorganisms for the Purposes of Patent Procedure. Since the strain referred to is being maintained under the terms of the Budapest Treaty, it will be made available to a patent office signatory to the Budapest Treaty.

The present invention also encompasses variants of the C/CLP antibodies of the invention (e.g., 171.204, 317.171, 149.159, 171.159, 4F8, 128.148, Z1, Z4, Z8, M1, M5, 1903, 102 and 204) comprising one or more amino acid residue substitutions in the variable light (V_{L}) domain and/or variable heavy (V_{H}) domain. The present invention also encompasses variants of the C/CLP antibodies of the invention (e.g., 171.204, 317.171, 149.159, 171.159, 4F8, 128.148, Z1, Z4, Z8, M1, M5, 1903, 102 and 204) with one or more additional amino acid residue substitutions in one or more V_{L} CDRs and/or one or more V_{H} CDRs. The antibody generated by introducing substitutions in the V_{H} domain, V_{H} CDRs, V_{L} domain and/or V_{L} CDRs of the C/CLP antibodies of the invention (e.g., 171.204, 317.171, 149.159, 171.159, 4F8, 128.148, Z1, Z4, Z8, M1, M5, 1903, 102 and 204) can be tested in vitro and in vivo, for example, for its ability to bind to a C/CLP (by, e.g., immunoassays including, but not limited to ELISA and BIAcore), or for its ability to antagonize one or more C/CLP.

It will be understood that the complementarity determining regions (CDRs) residue numbers referred to herein are those of Kabat et al. (1991, NIH Publication 91-3242, National Technical Information Service, Springfield, Va.). Specifically, residues 24-34 (CDR1), 50-56 (CDR2) and 89-97 (CDR3) in the light chain variable domain and 31-35 (CDR1), 50-65 (CDR2) and 95-102 (CDR3) in the heavy chain variable domain. Note that CDRs vary considerably from antibody to antibody (and by definition will not exhibit homology with the Kabat consensus sequences). Maximal alignment of framework residues frequently requires the insertion of “spacer” residues in the numbering system, to be used for the Fv region. It will be understood that the CDRs referred to herein are those of Kabat et al. supra. In addition, the identity of certain individual residues at any given Kabat site number may vary from antibody chain to antibody chain due to interspecies or allelic divergence.

Another embodiment of the present invention includes the introduction of conservative amino acid substitutions in any portion of a C/CLP antibody of interest, described supra (see Table 2). It is well known in the art that “conservative amino acid substitution” refers to amino acid substitutions that substitute functionally-equivalent amino acids. Conservative amino acid changes result in silent changes in the amino acid sequence of the resulting peptide. For example, one or more amino acids of a similar polarity act as functional equivalents and result in a silent alteration within the amino acid sequence of the peptide. Substitutions that are charge neutral and which replace a residue with a smaller residue may also be considered “conservative substitutions” even if the residues are in different groups (e.g., replacement of phenylalanine with the smaller isoleucine). Families of amino acid residues having similar side chains have been defined in the art. Several families of conservative amino acid substitutions are shown in Table 3.

Standard techniques known to those of skill in the art can be used to introduce mutations (e.g., additions, deletions, and/or substitutions) in the nucleotide sequence encoding a C/CLP antibody of the invention, including, for example, site-directed mutagenesis and PCR-mediated mutagenesis are routinely used to generate amino acid substitutions. In one embodiment, the derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions in the relative to the original C/CLP antibody. In another embodiment, the derivatives of a C/CLP antibody of the invention have conservative amino acid substitutions (e.g. supra) are made at one or more predicted non-essential amino acid residues (i.e., amino acid residues which are not critical for the antibody to specifically bind to a C/CLP). Alternatively, mutations can be introduced randomly along all or part of the coding sequence, such as by saturation mutagenesis, and the resultant mutants can be screened for biological activity to identify mutants that retain activity. Following mutagenesis, the encoded antibody can be expressed and the activity of the antibody can be determined.

The term “conservative amino acid substitution” also refers to the use of amino acid analogs or variants. Guidance concerning how to make phenotypically silent amino acid substitutions is provided in Bowie et al., “Deciphering the Message in Protein Sequences: Tolerance to Amino Acid Substitutions,” (1990, Science 247:1306-1310).

TABLE 2
Binding Characteristics of Some Specific C/CLP Antibodies

| | | | | | mAMCase | mAMCAse | mAMCase | |
|---|---|---|---|---|---|---|---|---|
| Antibody | Isotype | Origin | Antigen | mAMCase | TR | D136A | E140A | mChT |
| M5 | IgG2a | Human | N/A | +++ | Neg | +++ | +++ | Neg |
| M1^{E} | IgG2a | Human | N/A | ++++ | Neg | ++++ | ++++ | Neg |
| Z8^{A,B} | IgG2a | Human | N/A | + | Neg | ++ | + | Neg |
| Z4 | IgG2a | Human | N/A | Neg | Neg | Neg | Neg | Neg |
| Z1 | IgG2a | Human | N/A | ++ | ++ | +/− | + | ++++ |
| 171.159 | IgG2a | Rat | h/m | Neg | Neg | Neg | Neg | Neg |
| | | | AMCase | | | | | |
| 149.159 | IgG2a | Rat | h/m | Neg | Neg | Neg | Neg | Neg |
| | | | AMCase | | | | | |
| 128.148 | IgG2a | Rat | h/m | ++++ | ++++ | ++++ | ++++ | Neg |
| | | | AMCase | | | | | |
| 4F8^{E} | IgG1 | Hamster | mAMCase | ++++ | ++++ | ++++ | ++++ | Neg |
| 317.171 | IgG2b | Mouse | hAMCase | Neg | Neg | Neg | Neg | Neg |
| 171.204^{A, B, E} | IgG2b | Mouse | hAMCase | ++++ | Neg | ++++ | ++++ | + |

| | | | mYKL40/ | | hAMCase | | | |
|---|---|---|---|---|---|---|---|---|
| Antibody | Ym1 | Ym2 | BRP39 | hAMCase | TR | hChT | hYKL-39 | hYKL-40 |
| M5 | Neg | +/− | Neg | Neg | Neg | Neg | Neg | Neg |
| M1^{E} | Neg | Neg | Neg | Neg | Neg | Neg | Neg | Neg |
| Z8^{A,B} | Neg | Neg | Neg | Neg | Neg | Neg | Neg | Neg |
| Z4 | Neg | Neg | Neg | ++++ | Neg | Neg | Neg | Neg |
| Z1 | + | + | Neg | ++++ | ++++ | ++++ | + | Neg |
| 171.159 | Neg | Neg | Neg | +++ | ++++ | Neg | Neg | Neg |
| 149.159 | Neg | Neg | Neg | +++ | ++++ | Neg | +++ | + |
| 128.148 | Neg | Neg | Neg | Neg | Neg | Neg | Neg | Neg |
| 4F8^{E} | Neg | ++ | Neg | Neg | Neg | Neg | Neg | Neg |
| 317.171 | Neg | Neg | Neg | ++++ | Neg | Neg | Neg | Neg |
| 171.204^{A, B, E} | Neg | + | Neg | ++++ | Neg | +/− | Neg | Neg |

| | | | | | | mAMCase | mAMCase | |
|---|---|---|---|---|---|---|---|---|
| Antibody | Isotype | Origin | Antigen | mAMCase | mAMCase TR | D136A | E140A | mChT |
| 206^{B, C} | poly^{F} | Rabbit | Mouse | 20.48M | | | | 256K |
| | | | AMCase | | | | | |
| 208^{D} | poly | Rabbit | Mouse | | 102.4K | >204.8K | >204.8K | 3.2M |
| | | | ChT | | | | | |
| 212^{B} | poly | Rabbit | Mouse | | | | | |
| | | | YKL-40 | | | | | |
| 2870^{B} | poly | Rabbit | YM-1 | 64K | 204.8K | >204.8K | 204.8K | 16K |
| 655 | poly | Rabbit | Human | 320K | | | | 8K |
| | | | AMCase | | | | | |
| 657 | poly | Rabbit | Human | 3.2K | | | | 128K |
| | | | ChT | | | | | |
| 204 | poly | Rabbit | Human | 80K | | | | 32K |
| | | | YKL-40 | | | | | |

| Antibody | Ym1 | Ym2 | mYKL40/BRP39 | hAMCase | hChT | hYKL-39 | hYKL-40 |
|---|---|---|---|---|---|---|---|
| 206^{B, C} | 256K | 256K | 16K | 1.28M | 102K | 64K | 52K |
| 208^{D} | | 102.4 | 51.2 | 200K | >102K | >3.2K | >3.2K |
| 212^{B} | | | 314K | | | | |
| 2870^{B} | >512K | >512K | <1K | 512K | <1K | <1K | 1.6K |
| 655 | 64K | 64K | 2K | 4M | 4K | 16K | 1.6K |
| 657 | 2K | 1K | <1K | 4K | 1M | 2K | <1K |
| 204 | 32K | 32K | 128K | 26K | 16K | 52K | 400K |

++++ 4.00 OD at 62.5 ng/ml
+++ 2.50 to 3.99 OD at 62.5 ng/ml
++ 1.00 to 2.49 OD at 62.5 ng/ml
+ 0.20 to 0.99 OD at 62.5 ng/ml
+/− 0.30 to 1.0 OD at 1000 ng/ml
See FIG. 7 for binding competition maps
^{A}Inhibits cellular infiltration in allergen-induced asthma in vivo, See, Example 3
^{B}Inhibits IL-13 mediated AHR in vivo, See, Examples 3 & 6
Values indicate the minimal titer for which a signal is seen by ELISA; K = 1,000; M = 1,000,000
^{C}Inhibits AMCase chitinase activity by 4-Mu-chitotrioside assay in vitro, See, Example 2
^{D}Inhibits ChT chitinase activity by 4-Mu-chitotrioside assay in vitro, See, Example 2
^{E}Inhibits AMCase chitinase activity by chitin azure assay in vitro, See, Example 2
^{F}Poly = polyclonal

TABLE 3
Families of Conservative Amino Acid Substitutions

| Family | Amino Acids |
|---|---|
| non-polar | Trp, Phe, Met, Leu, Ile, Val, |
| | Ala, Pro |
| uncharged polar | Gly, Ser, Thr, Asn, Gln, Tyr, |
| | Cys |
| acidic/negatively charged | Asp, Glu |
| basic/positively charged | Arg, Lys, His |
| Beta-branched | Thr, Val, Ile |
| residues that influence chain orientation | Gly, Pro |
| aromatic | Trp, Tyr, Phe, His |

C/CLP antibodies of the invention may include, but are not limited to, synthetic antibodies, monoclonal antibodies, oligoclonal antibodies recombinantly produced antibodies, intrabodies, multispecific antibodies, bispecific antibodies, human antibodies, humanized antibodies, chimeric antibodies, synthetic antibodies, single-chain FvFcs (scFvFc), single-chain Fvs (scFv), and anti-idiotypic (anti-Id) antibodies. In particular, antibodies used in the methods of the present invention include immunoglobulin molecules and immunologically active portions of immunoglobulin molecules. The antibodies of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

C/CLP antibodies of the invention may be from any animal origin including birds and mammals (e.g., human, murine, donkey, sheep, rabbit, goat, guinea pig, camel, horse, or chicken). Preferably, the antibodies are human or humanized monoclonal antibodies. As used herein, “human” antibodies include antibodies having the amino acid sequence of a human immunoglobulin and include antibodies isolated from human immunoglobulin libraries or from mice that express antibodies from human genes.

Antibodies like all polypeptides have an Isoelectric Point (pI), which is generally defined as the pH at which a polypeptide carries no net charge. It is known in the art that protein solubility is typically lowest when the pH of the solution is equal to the isoelectric point (pI) of the protein. It is possible to optimize solubility by altering the number and location of ionizable residues in the antibody to adjust the pI. For example the pI of a polypeptide can be manipulated by making the appropriate amino acid substitutions (e.g., by substituting a charged amino acid such as a lysine, for an uncharged residue such as alanine). Without wishing to be bound by any particular theory, amino acid substitutions of an antibody that result in changes of the pI of said antibody may improve solubility and/or the stability of the antibody. One skilled in the art would understand which amino acid substitutions would be most appropriate for a particular antibody to achieve a desired pI. The pI of a protein may be determined by a variety of methods including but not limited to, isoelectric focusing and various computer algorithms (see for example Bjellqvist et al., 1993, Electrophoresis 14:1023). In one embodiment, the pI of the C/CLP antibodies of the invention is higher then about 6.5, about 7.0, about 7.5, about 8.0, about 8.5, or about 9.0. In another embodiment, the pI of the C/CLP antibodies of the invention is higher then 6.5, 7.0, 7.5, 8.0, 8.5, or 9.0. In one embodiment, substitutions resulting in alterations in the pI of the C/CLP antibody of the invention will not significantly diminish its binding affinity for a C/CLP. It is specifically contemplated that the substitution(s) of the Fc region that result in altered binding to FcγR (described supra) may also result in a change in the pI. In a preferred embodiment, substitution(s) of the Fc region are specifically chosen to effect both the desired alteration in FcγR binding and any desired change in pI. As used herein the pI value is defined as the pI of the predominant charge form. The pI of a protein may be determined by a variety of methods including but not limited to, isoelectric focusing and various computer algorithms (see, e.g., Bjellqvist et al., 1993, Electrophoresis 14:1023).

The Tm of the Fab domain of an antibody, can be a good indicator of the thermal stability of an antibody and may further provide an indication of the shelf-life. A lower Tm indicates more aggregation/less stability, whereas a higher Tm indicates less aggregation/more stability. Thus, antibodies having higher Tm are preferable. In one embodiment, the Fab domain of a C/CLP antibody has a Tm value higher than at least 50° C., 55° C., 60° C., 65° C., 70° C., 75° C., 80° C., 85° C., 90° C., 95° C., 100° C., 105° C., 110° C., 115° C. or 120° C. In another embodiment, the Fab domain of an C/CLP antibody has a Tm value higher than at least about 50° C., about 55° C., about 60° C., about 65° C., about 70° C., about 75° C., about 80° C., about 85° C., about 90° C., about 95° C., about 100° C., about 105° C., about 110° C., about 115° C. or about 120° C. Thermal melting temperatures (Tm) of a protein domain (e.g., a Fab domain) can be measured using any standard method known in the art, for example, by differential scanning calorimetry (see, e.g., Vermeer et al., 2000, Biophys. J. 78:394-404; Vermeer et al., 2000, Biophys. J. 79: 2150-2154).

C/CLP antibodies of the invention may be monospecific, bispecific, trispecific, or have greater multispecificity. Multispecific antibodies may specifically bind to different epitopes of desired target molecule or may specifically bind to both the target molecule as well as a heterologous epitope, such as a heterologous polypeptide or solid support material. See, e.g., International Publication Nos. WO 94/04690; WO 93/17715; WO 92/08802; WO 91/00360; and WO 92/05793; Tutt, et al., 1991, J. Immunol. 147:60-69; U.S. Pat. Nos. 4,474,893, 4,714,681, 4,925,648, 5,573,920, and 5,601,819; and Kostelny et al., 1992, J. Immunol. 148:1547). In one embodiment, one of the binding specificities is for a C/CLP, the other one is for any other antigen (i.e., another C/CLP, a signaling or effector molecule, etc.).

Multispecific antibodies have binding specificities for at least two different antigens. While such molecules normally will only bind two antigens (i.e. bispecific antibodies, BsAbs), antibodies with additional specificities such as trispecific antibodies are encompassed by the instant invention. Examples of BsAbs include without limitation those with one arm directed against a C/CLP and the other arm directed against any other antigen. Methods for making bispecific antibodies are known in the art. Traditional production of full-length bispecific antibodies is based on the coexpression of two immunoglobulin heavy chain-light chain pairs, where the two chains have different specificities (Millstein et al., 1983, Nature, 305:537-539). Because of the random assortment of immunoglobulin heavy and light chains, these hybridomas (quadromas) produce a potential mixture of different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., 1991, EMBO J., 10:3655-3659. A more directed approach is the generation of a Di-diabody a tetravalent bispecific antibody. Methods for producing a Di-diabody are known in the art (see e.g., Lu et al., 2003, J Immunol Methods 279:219-32; Marvin et al., 2005, Acta Pharmacolical Sinica 26:649).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. In one embodiment, the first heavy-chain constant region (CH1) containing the site necessary for light chain binding is present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when, the expression of at least two polypeptide chains in equal ratios results in high yields or when the ratios are of no particular significance.

In one embodiment of this approach, the bispecific antibodies are composed of a hybrid immunoglobulin heavy chain with a first binding specificity in one arm (e.g., a C/CLP), and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., 1986, Methods in Enzymology, 121:210. According to another approach described in W096/27011, a pair of antibody molecules can be engineered to maximize the percentage of heterodimers which are recovered from recombinant cell culture. The preferred interface comprises at least a part of the CH3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089. Heteroconjugate antibodies may be made using any convenient cross-linking methods. Suitable cross-linking agents are well known in the art, and are disclosed in U.S. Pat. No. 4,676,980, along with a number of cross-linking techniques.

Antibodies with more than two valencies incorporating at least one hinge modification of the invention are contemplated. For example, trispecific antibodies can be prepared. See, e.g., Tutt et al. J. Immunol. 147: 60 (1991).

The C/CLP antibodies of the invention encompass single domain antibodies, including camelized single domain antibodies (see e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079).

Other antibodies specifically contemplated are “oligoclonal” antibodies. As used herein, the term “oligoclonal” antibodies” refers to a predetermined mixture of distinct monoclonal antibodies. See, e.g., PCT publication WO 95/20401; U.S. Pat. Nos. 5,789,208 and 6,335,163. Preferably oligoclonal antibodies consist of a predetermined mixture of antibodies against one or more epitopes are generated in a single cell. More preferably oligoclonal antibodies comprise a plurality of heavy chains capable of pairing with a common light chain to generate antibodies with multiple specificities (e.g., PCT publication WO 04/009618). Oligoclonal antibodies are particularly useful when it is desired to target multiple epitopes on a single target molecule (e.g., a single C/CLP). Those skilled in the art will know or can determine what type of antibody or mixture of antibodies is applicable for an intended purpose and desired need.

It is specifically contemplated that antibody-like and antibody-domain fusion proteins may also be C/CLP antagonists of the present invention. An antibody-like molecule is any molecule that has been generated with a desired binding property, see, e.g., PCT Publication Nos. WO 04/044011; WO 04/058821; WO 04/003019 and WO 03/002609. Antibody-domain fusion proteins may incorporate one or more antibody domains such as the Fc domain or the variable domain. For example, the heterologous polypeptides may be fused or conjugated to a Fab fragment, Fd fragment, Fv fragment, F(ab)₂ fragment, a VH domain, a VL domain, a VH CDR, a VL CDR, or fragment thereof. A large number of antibody-domain molecules are known in the art including, but not limited to, diabodies (dsFv)₂ (Bera et al., 1998, J. Mol. Biol. 281:475-83); minibodies (homodimers of scFv-CH3 fusion proteins) (Pessi et al., 1993, Nature 362:367-9), tetravalent di-diabody (Lu et al., 2003 J. Immunol. Methods 279:219-32), tetravalent bi-specific antibodies called Bs(scFv)4-IgG (Zuo et al., 2000, Protein Eng. 13:361-367). Fc domain fusions combine the Fc region of an immunoglobulin with a fusion partner which in general can be an protein, including, but not limited to, a ligand, an enzyme, the ligand portion of a receptor, an adhesion protein, or some other protein or domain. See, e.g., Chamow et al., 1996, Trends Biotechnol 14:52-60; Ashkenazi et al., 1997, Curr Opin Immunol 9:195-200; Heidaran et al., 1995, FASEB J. 9:140-5. Methods for fusing or conjugating polypeptides to antibody portions are well known in the art. See, e.g., U.S. Pat. Nos. 5,336,603, 5,622,929, 5,359,046, 5,349,053, 5,447,851, and 5,112,946; European Patent Nos. EP 307,434 and EP 367,166; PCT Publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341.

In one embodiment, antibodies of the present invention also encompass C/CLP antibodies that have half-lives (e.g., serum half-lives) in a mammal, preferably a human, of greater than 5 days, greater than 10 days, greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. In another embodiment, antibodies of the present invention also encompass C/CLP antibodies that have half-lives (e.g., serum half-lives) in a mammal, preferably a human, of greater than about 5 days, greater than about 10 days, greater than about 15 days, preferably greater than about 20 days, greater than about 25 days, greater than about 30 days, greater than about 35 days, greater than about 40 days, greater than about 45 days, greater than about 2 months, greater than about 3 months, greater than about 4 months, or greater than about 5 months. The increased half-lives of the antibodies of the present invention in a mammal, preferably a human, results in a higher serum titer of said antibodies or antibody fragments in the mammal, and thus, reduces the frequency of the administration of said antibodies or antibody fragments and/or reduces the concentration of said antibodies or antibody fragments to be administered. Antibodies having increased in vivo half-lives can be generated by techniques known to those of skill in the art. For example, antibodies with increased in vivo half-lives can be generated by modifying (e.g., substituting, deleting or adding) amino acid residues identified as involved in the interaction between the Fc domain and the FcRn receptor (see, e.g., International Publication Nos. WO 97/34631; WO 04/029207; U.S. Pat. No. 6,737056 and U.S. Patent Publication No. 2003/0190311).

In one embodiment, the C/CLP antibodies of the invention may be chemically modified (e.g., one or more chemical moieties can be attached to the antibody) or be modified to alter its glycosylation, again to alter one or more functional properties of the antibody.

In still another embodiment, the glycosylation of the C/CLP antibodies of the invention is modified. For example, an aglycoslated antibody can be made (i.e., the antibody lacks glycosylation). Glycosylation can be altered to, for example, increase the affinity of the antibody for a target antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861.

Additionally or alternatively, a C/CLP antibody can be made that has an altered type of glycosylation, such as a hypofucosylated antibody having reduced amounts of fucosyl residues or an antibody having increased bisecting GlcNAc structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. See, for example, Shields, R. L. et al. (2002) J. Biol. Chem. 277:26733-26740; Umana et al. (1999) Nat. Biotech. 17:176-1, as well as, European Patent No: EP 1,176,195; PCT Publications WO 03/035835; WO 99/54342.

In still another embodiment, the glycosylation of a C/CLP antibody of the invention is modified. For example, an aglycoslated antibody can be made (i.e., the antibody lacks glycosylation). Glycosylation can be altered to, for example, increase the affinity of the antibody for a target antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861.

Additionally or alternatively, a C/CLP antibody can be made that has an altered type of glycosylation, such as a hypofucosylated C/CLP antibody having reduced amounts of fucosyl residues or a C/CLP antibody having increased bisecting GlcNAc structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. See, for example, Shields, R. L. et al. (2002) J. Biol. Chem. 277:26733-26740; Umana et al. (1999) Nat. Biotech. 17:176-1, as well as, European Patent No: EP 1,176,195; PCT Publications WO 03/035835; WO 99/54342.

In one embodiment, the C/CLP antibodies of the present invention may comprise modifications/substations and/or novel amino acids within their Fc domains such as, for example, those disclosed in Ghetie et al., 1997, Nat Biotech. 15:637-40; Duncan et al, 1988, Nature 332:563-564; Lund et al., 1991, J. Immunol 147:2657-2662; Lund et al, 1992, Mol Immunol 29:53-59; Alegre et al, 1994, Transplantation 57:1537-1543; Hutchins et al., 1995, Proc Natl. Acad Sci USA 92:11980-11984; Jefferis et al, 1995, Immunol Lett. 44:111-117; Lund et al., 1995, Faseb J 9:115-119; Jefferis et al, 1996, Immunol Lett 54:101-104; Lund et al, 1996, J Immunol 157:4963-4969; Armour et al., 1999, Eur J Immunol 29:2613-2624; Idusogie et al, 2000, J Immunol 164:4178-4184; Reddy et al, 2000, J Immunol 164:1925-1933; Xu et al., 2000, Cell Immunol 200:16-26; Idusogie et al, 2001, J Immunol 166:2571-2575; Shields et al., 2001, J Biol Chem 276:6591-6604; Jefferis et al, 2002, Immunol Lett 82:57-65; Presta et al., 2002, Biochem Soc Trans 30:487-490); U.S. Pat. Nos. 5,624,821; 5,885,573; 5,677,425; 6,165,745; 6,277,375; 5,869,046; 6,121,022; 5,624,821; 5,648,260; 6,194,551; 6,737,056; 6,821,505; 6,277,375; U.S. patent application Ser. No. 10/370,749 and PCT Publications WO 94/2935; WO 99/58572; WO 00/42072; WO 01/58957; WO 02/060919; WO 04/029207; WO 04/016750; WO 04/035752; WO 04/074455; WO 04/099249; WO 04/063351; WO 05/070963; WO 05/040217, WO 05/092925 and WO 06/020114. Other modifications/substitutions of the Fc domain will be readily apparent to one skilled in the art.

C/CLP antibodies of the invention comprising modifications/substitutions and/or novel amino acid residues in their Fc regions can be generated by numerous methods well known to one skilled in the art. Non-limiting examples include, isolating antibody coding regions (e.g., from hybridoma) and making one or more desired substitutions in the Fc region of the isolated antibody coding region. Alternatively, the variable regions of a C/CLP antibody may be subcloned into a vector encoding an Fc region comprising one or modifications/substations and/or novel amino acid residues.

### 5.5 Antibody Conjugates and Derivatives

C/CLP antibodies of the invention include derivatives that are modified (i.e., by the covalent attachment of any type of molecule to the antibody such that covalent attachment). For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

Antibodies or fragments thereof with increased in vivo half-lives can be generated by attaching to said antibodies or antibody fragments polymer molecules such as high molecular weight polyethyleneglycol (PEG). PEG can be attached to said antibodies or antibody fragments with or without a multifunctional linker either through site-specific conjugation of the PEG to the N- or C-terminus of said antibodies or antibody fragments or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography.

Further, antibodies can be conjugated to albumin in order to make the antibody or antibody fragment more stable in vivo or have a longer half life in vivo. The techniques are well known in the art, see e.g., International Publication Nos. WO 93/15199, WO 93/15200, and WO 01/77137; and European Patent No. EP 413622. The present invention encompasses the use of antibodies or fragments thereof conjugated or fused to one or more moieties, including but not limited to, peptides, polypeptides, proteins, fusion proteins, nucleic acid molecules, small molecules, mimetic agents, synthetic drugs, inorganic molecules, and organic molecules.

In one embodiment, the present invention encompasses the use of antibodies or fragments thereof recombinantly fused or chemically conjugated (including both covalent and non-covalent conjugations) to a heterologous protein or polypeptide (or fragment thereof, preferably to a polypeptide of at least 10, at least 20, at least 30, at least 40, at least 50, at least 60, at least 70, at least 80, at least 90 or at least 100 amino acids) to generate fusion proteins. In another embodiment, the present invention encompasses the use of antibodies or fragments thereof recombinantly fused or chemically conjugated (including both covalent and non-covalent conjugations) to a heterologous protein or polypeptide (or fragment thereof, preferably to a polypeptide of at least about 10, at least about 20, at least about 30, at least about 40, at least about 50, at least about 60, at least about 70, at least about 80, at least about 90 or at least about 100 amino acids) to generate fusion proteins. The fusion does not necessarily need to be direct, but may occur through linker sequences. For example, antibodies may be used to target heterologous polypeptides to particular cell types, either in vitro or in vivo, by fusing or conjugating the antibodies to antibodies specific for particular cell surface receptors. Antibodies fused or conjugated to heterologous polypeptides may also be used in in vitro immunoassays and purification methods using methods known in the art. See e.g., International publication No. WO 93/21232; European Patent No. EP 439,095; Naramura et al., 1994, Immunol. Lett. 39:91-99; U.S. Pat. No. 5,474,981; Gillies et al., 1992, PNAS 89:1428-1432; and Fell et al., 1991, J. Immunol. 146:2446-2452.

The present invention further includes formulations comprising heterologous proteins, peptides or polypeptides fused or conjugated to antibody fragments. For example, the heterologous polypeptides may be fused or conjugated to a Fab fragment, Fd fragment, Fv fragment, F(ab)2 fragment, a VH domain, a VL domain, a VH CDR, a VL CDR, or fragment thereof. Methods for fusing or conjugating polypeptides to antibody portions are well known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341.

Additional fusion proteins, e.g., of antibodies that specifically bind a C/CLP (e.g., supra), may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or fragments thereof with higher affinities and lower dissociation rates). See, generally, U.S. Pat. Nos. 5,605,793; 5,811,238; 5,830,721; 5,834,252; and 5,837,458, and Patten et al., 1997, Curr. Opinion Biotechnol. 8:724-33; Harayama, 1998, Trends Biotechnol. 16(2): 76-82; Hansson, et al., 1999, J. Mol. Biol. 287:265-76; and Lorenzo and Blasco, 1998, Biotechniques 24(2): 308-313. Antibodies or fragments thereof, or the encoded antibodies or fragments thereof, may be altered by being subjected to random mutagenesis by error-prone PCR, random nucleotide insertion or other methods prior to recombination. One or more portions of a polynucleotide encoding an antibody or antibody fragment, which portions specifically bind to a C/CLP may be recombined with one or more components, motifs, sections, parts, domains, fragments, etc. of one or more heterologous molecules.

Moreover, the antibodies or fragments thereof can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

In other embodiments, C/CLP antibodies of the present invention or analogs or derivatives thereof are conjugated to a diagnostic or detectable agent. Such antibodies can be useful for monitoring or prognosing the development or progression of a cancer as part of a clinical testing procedure, such as determining the efficacy of a particular therapy. Such diagnosis and detection can be accomplished by coupling the antibody to detectable substances including, but not limited to various enzymes, such as but not limited to horseradish peroxidase, alkaline phosphatase, beta-galactosidase, or acetylcholinesterase; prosthetic groups, such as but not limited to streptavidinlbiotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to iodine (131I, 125I, 123I, 121I,), carbon (14C), sulfur (35S), tritium (3H), indium (115In, 113In, 112In, 111In,), and technetium (99Tc), thallium (201Ti), gallium (68Ga, 67Ga), palladium (103Pd), molybdenum (99Mo), xenon (133Xe), fluorine (18F), 153Sm, 177Lu, 159Gd, 149Pm, 140La, 175Yb, 166Ho, 90Y, 47Sc, 186Re, 188Re,142 Pr, 105Rh, 97Ru, 68Ge, 57Co, 65Zn, 85Sr, 32P, 153Gd, 169Yb, 51Cr, 54Mn, 75Se, 113Sn, and 117Tin; positron emitting metals using various positron emission tomographies, noradioactive paramagnetic metal ions, and molecules that are radiolabelled or conjugated to specific radioisotopes.

The present invention further encompasses uses of C/CLP antibodies of the invention or fragments thereof conjugated to a therapeutic agent.

An antibody or fragment thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Examples include paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). A more extensive list of therapeutic moieties can be found in PCT publications WO 03/075957.

Further, a C/CLP antibody or fragment thereof may be conjugated to a therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, for example, a toxin such as abrin, ricin A, Onconase (or another cytotoxic RNase), pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Immunol., 6:1567), and VEGI (see, International Publication No. WO 99/23105), a thrombotic agent or an anti-angiogenic agent, e.g., angiostatin or endostatin; or, a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”)), or a growth factor (e.g., growth hormone (“GH”)).

Moreover, a C/CLP antibody can be conjugated to therapeutic moieties such as a radioactive materials or macrocyclic chelators useful for conjugating radiometal ions (see above for examples of radioactive materials). In certain embodiments, the macrocyclic chelator is 1,4,7,10-tetraazacyclododecane-N,N′,N″,N″-tetraacetic acid (DOTA) which can be attached to the antibody via a linker molecule. Such linker molecules are commonly known in the art and described in Denardo et al., 1998, Clin Cancer Res. 4:2483; Peterson et al., 1999, Bioconjug. Chem. 10:553; and Zimmerman et al., 1999, Nucl. Med. Biol. 26:943.

Techniques for conjugating therapeutic moieties to antibodies are well known. Moieties can be conjugated to antibodies by any method known in the art, including, but not limited to aldehyde/Schiff linkage, sulphydryl linkage, acid-labile linkage, cis-aconityl linkage, hydrazone linkage, enzymatically degradable linkage (see generally Garnett, 2002, Adv Drug Deliv Rev 53:171). Techniques for conjugating therapeutic moieties to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies ‘84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119.

Methods for fusing or conjugating antibodies to polypeptide moieties are known in the art. See, e.g., U.S. Pat. Nos. 5,336,603; 5,622,929; 5,359,046; 5,349,053; 5,447,851, and 5,112,946; EP 307,434; EP 367,166; PCT Publications WO 96/04388 and WO 91/06570; Ashkenazi et al., 1991, PNAS USA 88:10535; Zheng et al., 1995, J lmmunol 154:5590; and Vil et al., 1992, PNAS USA 89:11337. The fusion of an antibody to a moiety does not necessarily need to be direct, but may occur through linker sequences. Such linker molecules are commonly known in the art and described in Denardo et al., 1998, Clin Cancer Res 4:2483; Peterson et al., 1999, Bioconjug Chem 10:553; Zimmerman et al., 1999, Nucl Med Biol 26:943; Garnett, 2002, Adv Drug Deliv Rev 53:171.

Alternatively, an antibody can be conjugated to a second antibody to form an antibody heteroconjugate as described by Segal in U.S. Pat. No. 4,676,980.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

The therapeutic moiety or drug conjugated to an antibody or fragment thereof that specifically binds to a C/CLP of the invention should be chosen to achieve the desired prophylactic or therapeutic effect(s) for a particular disorder in a subject. A clinician or other medical personnel should consider the following when deciding on which therapeutic moiety or drug to conjugate to an antibody or fragment thereof that specifically binds to a C/CLP: the nature of the disease, the severity of the disease, and the condition of the subject.

### 5.6 Methods of Generating Antibodies

The C/CLP antibodies of the invention can be produced by any method known in the art for the synthesis of antibodies, in particular, by chemical synthesis or by recombinant expression techniques.

Polyclonal antibodies to a C/CLP can be produced by various procedures well known in the art. For example, a C/CLP or immunogenic fragments thereof can be administered to various host animals including, but not limited to, rabbits, mice, rats, etc. to induce the production of sera containing polyclonal antibodies specific for a C/CLP. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a C/CLP or a domain thereof (e.g., the extracellular domain) and once an immune response is detected, e.g., antibodies specific for a C/CLP are detected in the mouse serum, the mouse spleen is harvested and splenocytes isolated. The splenocytes are then fused by well known techniques to any suitable myeloma cells, for example cells from cell line SP20 available from the ATCC. Additionally, a RIMMS (repetitive immunization, multiple sites) technique can be used to immunize an animal (Kilpatrick et al., 1997, Hybridoma 16:381-9, incorporated herein by reference in its entirety). Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, monoclonal antibodies can be generated by culturing a hybridoma cell secreting an antibody, wherein the hybridoma is generated by fusing splenocytes isolated from a mouse immunized with a C/CLP or immunogenic fragments thereof, with myeloma cells and then screening the hybridomas resulting from the fusion for hybridoma clones that secrete an antibody able to bind a C/CLP.

Antibody fragments that recognize specific a C/CLP epitopes may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles that carry the polynucleotide sequences encoding them. In particular, DNA sequences encoding VH and VL domains are amplified from animal cDNA libraries (e.g., human or murine cDNA libraries of lymphoid tissues). The DNA encoding the VH and VL domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to the a C/CLP epitope of interest can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT Publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in International Publication No. WO 92/22324; Mullinax et al., 1992, BioTechniques 12(6): 864-869; Sawai et al., 1995, AJRI 34:26-34; and Better et al., 1988, Science 240:1041-1043.

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma constant, and the PCR amplified VL domains can be cloned into vectors expressing a VL constant region, e.g., human kappa or lamba constant regions. In one embodiment, the constant region is an Fc region containing at least one high effector function amino acid. In another embodiment, the vectors for expressing the VH or VL domains comprise a promoter, a secretion signal, a cloning site for both the variable and constant domains, as well as a selection marker such as neomycin. The VH and VL domains may also be cloned into one vector expressing the desired constant regions. The heavy chain conversion vectors and light chain conversion vectors are then co-transfected into cell lines to generate stable or transient cell lines that express full-length antibodies, e.g., IgG, using techniques known to those of skill in the art.

A chimeric antibody is a molecule in which different portions of the antibody are derived from different immunoglobulin molecules. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816397, and 6,311,415

For some uses, including in vivo use of antibodies in humans and in vitro detection assays, it may be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and PCT Publication Nos. WO 98/46645, WO 98/50433, WO 98/24893, WO98/16654, WO 96/34096, WO 96/33735, and WO 91/10741.

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immunoglobulin. A humanized antibody comprises substantially all of at least one, and typically two, variable domains (Fab, Fab′, F(ab′)2, Fabc, Fv) in which all or substantially all of the CDR regions correspond to those of a non-human immunoglobulin (i.e., donor antibody) and all or substantially all of the framework regions are those of a human immunoglobulin consensus sequence. In one embodiment, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG1, IgG2, IgG3 and lgG4. Usually the constant domain is a complement fixing constant domain where it is desired that the humanized antibody exhibit cytotoxic activity, and the class is typically IgG.sub.1. Where such cytotoxic activity is not desirable, the constant domain may be of the IgG.sub.2 class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. In one embodiment, at least 75% of the humanized antibody residues will correspond to those of the parental framework region (FR) and CDR sequences. In another embodiment at least 90% of the humanized antibody residues will correspond to those of the parental framework region (FR) and CDR sequences. In a further embodiment, greater than 95% of the humanized antibody residues will correspond to those of the parental framework region (FR) and CDR sequences. In yet another embodiment, at least about 75% of the humanized antibody residues will correspond to those of the parental framework region (FR) and CDR sequences. In a further embodiment at least about 90% of the humanized antibody residues will correspond to those of the parental framework region (FR) and CDR sequences. In yet a further embodiment, greater than about 95% of the humanized antibody residues will correspond to those of the parental framework region (FR) and CDR sequences. Humanized antibody can be produced using variety of techniques known in the art, including but not limited to, CDR-grafting (European Patent No. EP 239,400; International Publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5): 489-498; Studnicka et al., 1994, Protein Engineering 7(6): 805-814; and Roguska et al., 1994, PNAS 91:969-973), chain shuffling (U.S. Pat. No. 5,565,332), and techniques disclosed in, e.g., U.S. Pat. No. 6,407,213, U.S. Pat. No. 5,766,886, WO 9317105, Tan et al., J. Immunol. 169:1119-25 (2002), Caldas et al., Protein Eng. 13(5): 353-60 (2000), Morea et al., Methods 20(3): 267-79 (2000), Baca et al., J. Biol. Chem. 272(16): 10678-84 (1997), Roguska et al., Protein Eng. 9(10): 895-904 (1996), Couto et al., Cancer Res. 55 (23 Supp): 5973s-5977s (1995), Couto et al., Cancer Res. 55(8): 1717-22 (1995), Sandhu JS, Gene 150(2): 409-10 (1994), and Pedersen et al., J. Mol. Biol. 235(3): 959-73 (1994). Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323).

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring that express human antibodies. The transgenic mice are immunized in the normal fashion with a selected antigen, e.g., a C/CLP or immunogenic fragments thereof. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598. In addition, companies such as Abgenix, Inc. (Freemont, Calif.), Genpharm (San Jose, Calif.) and Medarex (Princeton, N.J.) can be engaged to provide human antibodies directed against a selected antigen using technology similar to that described above.

Further, the antibodies of the invention can, in turn, be utilized to generate anti-idiotype antibodies that “mimic” a C/CLP using techniques well known to those skilled in the art. (See, e.g., Greenspan & Bona, 1989, FASEB J. 7(5): 437-444; and Nissinoff, 1991, J. Immunol. 147(8): 2429-2438). For example, antibodies of the invention which bind to and competitively inhibit the binding of a C/CLP (as determined by assays well known in the art and disclosed infra) to its ligands can be used to generate anti-idiotypes that “mimic” a C/CLP binding domains and, as a consequence, bind to and neutralize a C/CLP and/or its ligands. Such neutralizing anti-idiotypes or Fab fragments of such anti-idiotypes can be used in therapeutic regimens to neutralize a C/CLP. The invention provides methods employing the use of polynucleotides comprising a nucleotide sequence encoding an antibody of the invention or a fragment thereof.

In a preferred embodiment, the nucleotide sequence encoding an antibody that specifically binds a C/CLP is obtained and used to generate the C/CLP antibodies of the invention. The nucleotide sequence can be obtained from sequencing hybridoma clone DNA. If a clone containing a nucleic acid encoding a particular antibody or an epitope-binding fragment thereof is not available, but the sequence of the antibody molecule or epitope-binding fragment thereof is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from any tissue or cells expressing the antibody, such as hybridoma cells selected to express an antibody) by PCR amplification using synthetic primers that hybridize to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, Or example, the techniques described in Current Protocols in Molecular Biology, F. M. Ausubel et al., ed., John Wiley & Sons (Chichester, England, 1998); Molecular Cloning: A Laboratory Manual, 3rd Edition, J. Sambrook et al., ed., Cold Spring Harbor Laboratory Press (Cold Spring Harbor, N.Y., 2001); Antibodies: A Laboratory Manual, E. Harlow and D. Lane, ed., Cold Spring Harbor Laboratory Press (Cold Spring Harbor, N.Y., 1988); and Using Antibodies: A Laboratory Manual, E. Harlow and D. Lane, ed., Cold Spring Harbor Laboratory (Cold Spring Harbor, N.Y., 1999)), to generate antibodies having a different amino acid sequence by, for example, introducing deletions, and/or insertions into desired regions of the antibodies.

In a preferred embodiment, one or more substitutions are made within the Fc region (e.g. supra) of an antibody able to specifically bind a C/CLP. In one embodiment, the amino acid substitutions modify binding to one or more Fc ligands (e.g., FcγRs, C1q) and alter ADCC and/or CDC activity.

In a specific embodiment, one or more of the CDRs is inserted within framework regions using routine recombinant DNA techniques. The framework regions may be naturally occurring or consensus framework regions, and preferably human framework regions (see, e.g., Chothia et al., 1998, J. Mol. Biol. 278: 457-479 for a listing of human framework regions). In one embodiment, the polynucleotide generated by the combination of the framework regions and CDRs encodes an antibody that specifically binds to a C/CLP. In another embodiment, as discussed supra, one or more amino acid substitutions may be made within the framework regions, and, in yet another embodiment, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

### 5.7 Polynucleotides Encoding Antibodies

The invention further provides polynucleotides comprising a nucleotide sequence encoding a C/CLP antibody of the invention and fragments thereof. The invention also encompasses polynucleotides that hybridize under stringent or lower stringency hybridization conditions, e.g., as defined herein, to polynucleotides that encode a C/CLP antibody, preferably, that specifically binds to a C/CLP of the invention, preferably, an antibody that binds to a polypeptide having the amino acid sequence of human or mouse AMCase (SEQ ID NO:1 and 2, respectively). In another preferred embodiment, the antibody binds specifically to a polypeptide having the amino acid sequence of human or mouse chitotriosidase (e.g., SEQ ID NO:3 and 4, respectively). In another preferred embodiment, the antibody binds specifically to a polypeptide having the amino acid sequence of human or mouse YKL-40 (e.g., SEQ ID NO:5 and 6, respectively). In another embodiment, the antibody binds a polypeptide having the amino acid sequence of human or mouse Oviductin (e.g., SEQ ID NO:7 and 8, respectively). In still another embodiment, the antibody binds a polypeptide having the amino acid sequence of TSA1902-L or TSA 1902-S (e.g., SEQ ID NO:9 and 10, respectively). In yet another embodiment, the antibody binds a polypeptide having the amino acid sequence of mouse YM1 or YM2 (e.g., SEQ ID NO: 11 and 12, respectively). In other embodiments, the antibody binds a polypeptide having the amino acid sequence of human YKL-39 (e.g., SEQ ID NO: 13). In still other embodiments, the antibody binds a polypeptide having the amino acid sequence of human SI-CLP (e.g., SEQ ID NO: 14)

By “stringent hybridization conditions” is intended overnight incubation at 42.degree. C. in a solution comprising: 50% formamide, 5.times.SSC (750 mM NaCl, 75 mM trisodium citrate), 50 mM sodium phosphate (pH 7.6), 5 times. Denhardt's solution, 10% dextran sulfate, and 20 μg/ml denatured, sheared salmon sperm DNA, followed by washing the filters in 0.1.times.SSC at about 65.degree. C.

The polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably polyA+RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence and corresponding amino acid sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY), to generate antibodies having a different amino acid sequence, for example to create amino acid substitutions, deletions, and/or insertions.

In a specific embodiment, the amino acid sequence of the heavy and/or light chain variable domains of the antibodies of the invention may be inspected to identify the sequences of the complementarity determining regions (CDRs) by methods that are well known in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody, as described supra. The framework regions may be naturally occurring or consensus framework regions, and preferably human framework regions (see, e.g., Chothia et al., J. Mol. Biol. 278: 457-479 (1998) for a listing of human framework regions). Preferably, the polynucleotide generated by the combination of the framework regions and CDRs encodes an antibody that specifically binds a polypeptide of the invention.

Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies.

Alternatively, techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038-1041 (1988)).

### 5.8 Methods of Producing Antibodies

The antibodies of the invention can be produced by any method known in the art for the synthesis of antibodies, in particular, by chemical synthesis or preferably, by recombinant expression techniques.

Recombinant expression of an antibody of the invention, or fragment, derivative or analog thereof, (e.g., a heavy or light chain of an antibody of the invention or a single chain antibody of the invention), requires construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an antibody of the invention. Thus, the invention includes host cells containing a polynucleotide encoding an antibody of the invention, or a heavy or light chain thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NSO, 3T3, PerC6 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)). Also see, e.g., U.S. Pat. Nos. 5,827,739, 5,879,936, 5,981,216, and 5,658,759.

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lacZ coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, Proc. Natl. Acad. Sci. USA 81:355-359 (1984)). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, NSO, Per.C6 and in particular, breast cancer cell lines such as, for example, BT483, Hs578T, HTB2, BT20 and T47D, and normal mammary gland cell lines such as, for example, CRL7030 and Hs578Bst.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compounds that interact directly or indirectly with the antibody molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 (1980)) genes can be employed in tk-, hgprt- or aprt-cells, respectively. Also, antimetabolite resistance can be used as the basis of selection for the following genes: dhfr, which confers resistance to methotrexate (Wigler et al., Proc Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, 1993, TIB TECH 11(5):155-215); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981).

The expression levels of an antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 3:257 (1983)).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, Nature 322:562 (1986); Kohler, Proc. Natl. Acad. Sci. USA 77:2197 (1980)). The coding sequences for the heavy and light chains may comprise cDNA or genomic DNA.

Once an antibody molecule of the invention has been produced by an animal, chemically synthesized, or recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In addition, the antibodies of the present invention or fragments thereof can be fused to heterologous polypeptide sequences described herein or otherwise known in the art, to facilitate purification.

Moreover, the antibodies or fragments thereof of the present invention can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

### 5.9 Methods of Treatment

As discussed above, C/CLP antagonists can be utilized for the prevention, management, treatment or amelioration of an inflammatory condition or one or more symptoms thereof and/or the inhibition of IL-13 mediated inflammation. Accordingly, the present invention provides methods (referred to herein as “method(s) of the invention”) for use of the C/CLP antagonists of the invention for the prevention, management, treatment or amelioration of an inflammatory condition or one or more symptoms thereof and/or the inhibition of IL-13 mediated inflammation. In one embodiment, the present invention encompasses a method of treating an inflammatory condition or one or more symptoms thereof and/or the inhibition of IL-13 mediated inflammation comprising administering to a subject a C/CLP antagonist. In another embodiment, the methods of the invention comprise the administration to a subject of an effective amount of one or more C/CLP antagonist alone or in combination with other prophylactic or therapeutic agents. In a specific embodiment, a subject is a mammal. In another embodiment, a subject is a human.

The methods of the present invention are useful for the prevention, management, treatment or amelioration of an inflammatory condition, disease or disorder including, but are not limited to, interstitial lung disease (ILD), pulmonary fibrosis, bronchitis, chronic obstructive pulmonary disease (COPD), pneumonia, pneumonitis, acute respiratory distress syndrome (ARDS), severe acute respiratory syndrome (SARS), sarcoidosis, cystic fibrosis (CF), emphysema, asthma, smoker's cough, allergy, allergic rhinitis, sinusitis, Paget's disease, abnormal bone remodeling, osteoporosis, Gorham-Stout syndrome, osteoarthritis, rheumatoid arthritis, psoriatic arthritis and brittle bone disease.

In a specific embodiment, the methods of the invention comprise the administration of an effective amount of one or more C/CLP antagonist, to a subject in need thereof, resulting in an improvement (e.g., a reduction in severity) in one or more of the changes associated with inflammatory lung disease including, but not limited to, tissue inflammation, increased lung volume, increased eosinophils in bronchioalveolar ravage (BAL) fluid, increased lymphocytes in BAL fluid, increased total cells in BAL fluid, increased alveolus size, increased deposition of crystals comprising chitinase-like molecules in lung tissue, increased airway resistance, increased mucus metaplasia, increased mucin expression, increased parenchymal fibrosis, increased airway remodeling (e.g., airway thickening, mucus metaplasia, epithelial hypertrophy and airway fibrosis), increased subepithelial fibrosis, increased collagen deposition in airway tissue, epithelial hypertrophy in the lung tissue, focal organization of crystalline material into Masson body-like fibrotic foci, airway hyperresponsiveness (AHR), and the like.

In another specific embodiment, the methods of the invention comprise the administration of an effective amount of one or more C/CLP antagonist, to a subject in need thereof, resulting in a decrease or inhibition of the ability of a C/CLP to mediated inflammatory responses including, but not limited to, those mediated by IL-13, such as the production of MCP-1, MCP-2, MIP-1β, eotaxin, eotaxin-2 and ENA-78, and those mediated directly by a C/CLP, such as production of MCP-1 and eotaxin. In other embodiments, the methods of the invention comprise the administration of an effective amount of one or more C/CLP antagonist resulting in a decrease or inhibition of one or more C/CLP activity including, but not limited to, chitinolytic activity, saccharide (e.g., GlcNAc) binding activity, chemotatic activity, receptor binding activity, signal transduction activity, receptor binding activity.

It is contemplated that the methods detailed herein are not limited to treatment of an inflammatory disease once the disease is established. Particularly, the symptoms of the disease need not have manifested to the point of detriment to the subject; indeed, the disease need not be detected in a subject before treatment is administered. That is, significant pathology from an inflammatory disease does not have to occur before the present invention may provide benefit. Therefore, the present invention, as described more fully herein, includes a method for preventing an inflammatory disease in a subject, in that a C/CLP antagonist, as detailed herein, can be administered to a subject prior to the onset of an inflammatory disease, thereby preventing the disease as demonstrated by the data disclosed herein. Thus in one embodiment, the prevention of inflammatory disease encompasses administering to a subject a C/CLP antagonist as a preventative measure against inflammatory disease. As detailed herein, the symptoms and etiologies of C/CLP associated and/or IL-13 mediated inflammatory disease include tissue inflammation, increased lung volume, increased eosinophils in bronchioalveolar ravage (BAL) fluid, increased lymphocytes in BAL fluid, increased total cells in BAL fluid, increased alveolus size, increased deposition of crystals comprised of C/CLPs in lung tissue, increased airway resistance, increased mucus metaplasia, increased mucin expression, increased parenchymal fibrosis, increased airway remodeling, increased subepithelial fibrosis, increased collagen deposition in airway tissue, epithelial hypertrophy in the lung tissue, focal organization of crystalline material into Masson body-like fibrotic foci, and the like. Given these etiologies and the methods disclosed elsewhere herein, the skilled artisan can recognize and prevent an inflammatory disease in a subject using one or more C/CLP antagonists before the disease pathology can be detected. This is because the data disclosed herein demonstrate that administration of a C/CLP antagonist, including, but not limited to, the C/CLP antibodies disclosed herein, prevented onset of an inflammatory disease in a subject (e.g., allergen (e.g., ovalbumin sensitization) induced asthma). Accordingly, the skilled artisan would appreciate, based on the disclosure provided elsewhere herein, that the present invention includes a method of preventing disease comprising administering a C/CLP antagonist. Further, as more fully discussed elsewhere herein, methods of antagonizing (e g., inhibiting) a C/CLP encompass a wide plethora of techniques for inhibiting not only a C/CLP mediated activity, but also for inhibiting expression of a nucleic acid encoding a chitinase-like molecule. Additionally, as disclosed elsewhere herein, one skilled in the art would understand, once armed with the teaching provided herein, that the present invention encompasses a method of preventing a wide variety of diseases where expression and/or activity of a C/CLP mediates disease. Methods for assessing whether a disease relates to over expression or increased activity of a C/CLP are disclosed elsewhere herein and/or are well-known in the art. Further, the invention encompasses treatment or prevention of such diseases discovered in the future.

The invention further encompasses methods for treating an IL-13 mediated inflammatory disease. This is because, as is known in the art, IL-13 overexpression in the lungs, among other tissues, whether inducible or constitutive, mediates or is associated with the increased expression of C/CLP in respiratory tissues, leading to, among other things, the pathologies described elsewhere herein (see e.g., Webb et al., 2001, JBC 276:41969-76; Zhu et al., 2004, Science 304:1678-82; U.S. Patent Publication 2003/0049261). Thereby, the present invention includes methods of treating an IL-13 mediated inflammatory disease using the methods of the present invention.

The skilled artisan will appreciate that the present invention is not limited to these chitinase-like molecules or to these C/CLP antagonists, and the data disclosed herein amply demonstrate that antagonizing a C/CLP can effectively treat and/or prevent an inflammatory disease.

### 5.10 Formulations and Administration

As described above, the invention encompasses administration of a C/CLP antagonist, for the prevention, management, treatment or amelioration of an inflammatory condition or one or more symptoms thereof and/or the inhibition of IL-13 mediated inflammation. Accordingly, the present invention provides formulations (e.g., a pharmaceutical composition) comprising one or more C/CLP antagonist (referred to herein as “formulation(s) of the invention,” “compositions of the invention” or simply “formulation(s),” or “compositions”).

In one embodiment, formulations (e.g., a pharmaceutical composition) comprise one or more C/CLP and a pharmaceutically-acceptable carrier. As used herein, the term “pharmaceutically-acceptable carrier” means a chemical composition with which an appropriate C/CLP antagonist may be combined and which, following the combination, can be used to administer the appropriate C/CLP antagonist to a subject. In a specific embodiment, the formulations of the invention comprise a C/CLP antibody of the invention.

In a specific embodiment, formulations (e.g., a pharmaceutical composition) comprising one or more C/CLP and a pharmaceutically-acceptable carrier are liquid formulations. (referred to herein as “liquid formulation(s)” which are specifically encompassed by the more generic terms “formulation(s) of the invention” and “formulation(s)”). In a specific embodiment, the liquid formulations are substantially free of surfactant and/or inorganic salts. In another specific embodiment, the liquid formulations have a pH ranging from about 5.0 to about 7.0, about 5.5 to about 6.5, or about 5.8 to about 6.2, or about 6.0. In another specific embodiment, the liquid formulations have a pH ranging from 5.0 to 7.0, 5.5 to 6.5, or 5.8 to 6.2, or 6.0. In yet another specific embodiment, the liquid formulations comprise histidine at a concentration ranging from about 1 mM to about 100 mM, or from about 5 mM to about 50 mM, or about 10 mM to about 25 mM. In still another specific embodiment, the liquid formulations comprise histidine at a concentration ranging from 1 mM to 100 mM, or from 5 mM to 50 mM, or 10 mM to 25 mM.

It is contemplated that the liquid formulations may further comprise one or more excipients such as a saccharide, an amino acid (e.g. arginine, lysine, and methionine) and a polyol. Additional descriptions and methods of preparing and analyzed liquid formulations can be found, for example, in PCT publications WO 03/106644; WO 04/066957; WO 04/091658.

In one embodiment the formulations (e.g., liquid formulations) of the invention are pyrogen-free formulations which are substantially free of endotoxins and/or related pyrogenic substances. Endotoxins include toxins that are confined inside a microorganism and are released when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, it is advantageous to remove even low amounts of endotoxins from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in amounts of several hundred or thousand milligrams per kilogram body weight, as can be the case with monoclonal antibodies, it is advantageous to remove even trace amounts of endotoxin. In one embodiment, endotoxin and pyrogen levels in the composition are less then 10 EU/mg, or less then 5 EU/mg, or less then 1 EU/mg, or less then 0.1 EU/mg, or less then 0.01 EU/mg, or less then 0.001 EU/mg.

When used for in vivo administration, the compositions described herein should be sterile. This is readily accomplished, for example, by filtration through sterile filtration membranes or by other means well known in the art. Sterile compositions for injection can be formulated according to conventional pharmaceutical practice as described in Remington's Pharmaceutical Sciences (180' ed, Mack Publishing Company, Easton, Pa., 1990). Compositions comprising antibodies, such as those disclosed herein, ordinarily will be stored in lyophilized form or in solution. It is contemplated that sterile compositions comprising antibodies of the invention are placed into a container having a sterile access port, for example, an intravenous solution bag or vial having an adapter that allows retrieval of the formulation, such as a stopper pierceable by a hypodermic injection needle.

In another embodiment, the liquid formulations have a concentration of one or more C/CLP antagonist of about 50 mg/ml, about 75 mg/ml, about 100 mg/ml, about 125 mg/ml, about 150 mg/ml, about 175 mg/ml, about 200 mg/ml, about 225 mg/ml, about 250 mg/ml, about 275 mg/ml, or about 300 mg/ml. In another embodiment, the liquid formulations have a concentration of one or more C/CLP antagonist of 50 mg/ml, 75 mg/ml, 100 mg/ml, 125 mg/ml, 150 mg/ml, 175 mg/ml, 200 mg/ml, 225 mg/ml, 250 mg/ml, 275 mg/ml, or 300 mg/ml.

In a specific embodiment, the liquid formulations of the invention comprise a C/CLP antibody of the invention. In another specific embodiment, the liquid formulations should exhibit one, or more of the following characteristics, stability, low to undetectable levels of antibody fragmentation and/or aggregation, very little to no loss of the biological activities of the antibodies or antibody fragments during manufacture, preparation, transportation, and storage. In certain embodiments the liquid formulations lose less than 50%, or less than 30%, or less than 20%, or less than 10% or even less than 5% or 1% of the antibody activity within 1 year storage under suitable conditions at about 4° C. The activity of an antibody can be determined by a suitable antigen-binding or effector function assay for the respective antibody. In yet another embodiment, the liquid formulations are of low viscosity and turbidity. In a particular embodiment, the liquid formulations have a viscosity of less than 10.00 cP at any temperature in the range of 1 to 26° C. Viscosity can be determined by numerous methods well known in the art. For example, the viscosity of a polypeptide solution can be measured using a ViscoLab 4000 Viscometer System (Cambridge Applied Systems) equipped with a ViscoLab Piston (SN:7497, 0.3055″, 1-20 cP) and S6S Reference Standard (Koehler Instrument Company, Inc.) and connected to a water bath to regulate the temperature of the samples being analyzed. The sample is loaded into the chamber at a desired starting temperature (e.g., 2° C.) and the piston lowered into the sample. After sample was equilibrated to the temperature of the chamber, measurement is initiated. The temperature is increased at a desired rate to the desired final temperature (e.g., ≧25° C.). And the viscosity over time is recorded.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats and dogs, and birds including commercially relevant birds such as chickens, ducks, geese, and turkeys.

It will be apparent to one skilled in the art that a formulation comprising one or more C/CLP antagonist to be administered to a subject (e.g., a human) in need thereof should be formulated in a pharmaceutically-acceptable excipient. Examples of formulations, pharmaceutical compositions in particular, of the invention include but are not limited to those disclosed in PCT publications WO 02/070007, WO 03/075957 and WO 04/066957. Briefly, the excipient that is included with the Fc variants and/or variant Fc fusion of the present invention in these formulations (e.g., liquid formulations) can be selected based on the expected route of administration of the formulations in therapeutic applications. The route of administration of the formulations depends on the condition to be treated. For example, intravenous injection may be preferred for treatment of a systemic disorder. The dosage of the formulations to be administered can be determined by the skilled artisan without undue experimentation in conjunction with standard dose-response studies. Relevant circumstances to be considered in making those determinations include the condition or conditions to be treated, the choice of formulations to be administered, the age, weight, and response of the individual patient, and the severity of the patient's symptoms. For example, the actual patient body weight may be used to calculate the dose of the C/CLP antagonist of the present invention in these formulations in milliliters (mL) to be administered. There may be no downward adjustment to “ideal” weight. In such a situation, an appropriate dose may be calculated by the following formula:

Dose (mL)=[patient weight (kg)×dose level (mg/kg)/drug concentration (mg/mL)]

Depending on the condition, the formulations can be administered orally, parenterally, intramuscularly, intranasally, vaginally, rectally, lingually, sublingually, buccally, intrabuccally, intravenously, cutaneously, subcutaneously and/or transdermally to the patient.

Accordingly, formulations designed for oral, parenteral, intramuscular, intranasal, vaginal, rectal, lingual, sublingual, buccal, intrabuccal, intravenous, cutaneous, subcutaneous and/or transdermal administration can be made without undue experimentation by means well known in the art, for example, with an inert diluent or with an edible carrier. The formulations may be enclosed in gelatin capsules or compressed into tablets. For the purpose of oral therapeutic administration, the formulations of the present invention may be incorporated with excipients and used in the form of tablets, troches, capsules, elixirs, suspensions, syrups, wafers, chewing gums, and the like.

Tablets, pills, capsules, troches and the like may also contain binders, recipients, disintegrating agent, lubricants, sweetening agents, and/or flavoring agents. Some examples of binders include microcrystalline cellulose, gum tragacanth and gelatin. Examples of excipients include starch and lactose. Some examples of disintegrating agents include alginic acid, cornstarch, and the like. Examples of lubricants include magnesium stearate and potassium stearate. An example of a glidant is colloidal silicon dioxide. Some examples of sweetening agents include sucrose, saccharin, and the like. Examples of flavoring agents include peppermint, methyl salicylate, orange flavoring, and the like. Materials used in preparing these various formulations should be pharmaceutically pure and non-toxic in the amounts used.

The formulations of the present invention can be administered parenterally, such as, for example, by intravenous, intramuscular, intrathecal and/or subcutaneous injection. Parenteral administration can be accomplished by incorporating the formulations of the present invention into a solution or suspension. Such solutions or suspensions may also include sterile diluents, such as water for injection, saline solution, fixed oils, polyethylene glycols, glycerine, propylene glycol and/or other synthetic solvents. Parenteral formulations may also include antibacterial agents, such as, for example, benzyl alcohol and/or methyl parabens, antioxidants, such as, for example, ascorbic acid and/or sodium bisulfite, and chelating agents, such as EDTA. Buffers, such as acetates, citrates and phosphates, and agents for the adjustment of tonicity, such as sodium chloride and dextrose, may also be added. The parenteral preparation can be enclosed in ampules, disposable syringes and/or multiple dose vials made of glass or plastic. Rectal administration includes administering the formulation into the rectum and/or large intestine. This can be accomplished using suppositories and/or enemas. Suppository formulations can be made by methods known in the art. Transdermal administration includes percutaneous absorption of the formulation through the skin. Transdermal formulations include patches, ointments, creams, gels, salves, and the like. The formulations of the present invention can be administered nasally to a patient. As used herein, nasally administering or nasal administration includes administering the formulations to the mucous membranes of the nasal passage and/or nasal cavity of the patient.

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and NO may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylkydroxybenzoate. The droplets provided by this route of administration preferably have an average diameter in the range from about 0.1 to about 200 nanometers. Pulmonary drug delivery may be achieved by inhalation, and administration by inhalation herein may be oral and/or nasal. Examples of pharmaceutical devices for pulmonary delivery include metered dose inhalers (MDIs) and dry powder inhalers (DPIs). Exemplary delivery systems by inhalation which can be adapted for delivery of the subject antibody and/or active agent are described in, for example, U.S. Pat. Nos. 5,756,353; 5,858,784; and PCT applications WO98/31346; WO98/10796; WO00/27359; WO01/54664; WO02/060412. Other aerosol formulations that may be used for delivering the antibody and/or active agent are described in U.S. Pat. Nos. 6,294,153; 6,344,194; 6,071,497, and PCT applications WO02/066078; WO02/053190; WO01/60420; WO00/66206.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 30 micrometers. Such a formulation is administered in the manner in which snuff is taken i.e. by rapid inhalation through the nasal passage from a container of the powder held close to the nares.

In certain embodiments, the formulations (e.g., liquid formulations) are administered to the mammal by subcutaneous (i.e., beneath the skin) administration. For such purposes, the formulations may be injected using a syringe. However, other devices for administration of the formulations are available such as injection devices (e.g. the Inject-ease_ and Genject_{—} devices), injector pens (such as the GenPen™); auto-injector devices, needleless devices (e.g., MediJector and BioJector); and subcutaneous patch delivery systems.

In another aspect of the invention there is provided a slow release formulations. In a specific embodiment, a slow release formulation comprises a liquid formulation. Slow release formulations may be formulated from a number of agents including, but not limited to, polymeric nano or microparticles and gels (e.g., a hyaluronic acid gel). Besides convenience, slow release formulations offer other advantages for delivery of protein drugs including protecting the protein (e.g., C/CLP antibodies of the invention) over an extended period from degradation or elimination, and the ability to deliver the protein locally to a particular site or body compartment thereby lowering overall systemic exposure.

The present invention, for example, also contemplates injectable depot formulations in which the protein (e.g., C/CLP antibodies of the invention) is embedded in a biodegradable polymeric matrix. Polymers that may be used include, but are not limited to, the homo- and co-polymers of lactic and glycolic acid (PLGA). PLGA degrades by hydrolysis to ultimately give the acid monomers and is chemically unreactive under the conditions used to prepare, for example, microspheres and thus does not modify the protein. After subcutaneous or intramuscular injection, the protein is released by a combination of diffusion and polymer degradation. By using polymers of different composition and molecular weight, the hydrolysis rate can be varied thereby allowing release to last from days to months. In a further aspect the present invention provides a nasal spray formulation. In a specific embodiment, a nasal spray formulation comprises the liquid formulation of the present invention.

The formulations of the invention may be used in accordance with the methods of the invention for the prevention, management, treatment or amelioration of an inflammatory condition or one or more symptoms thereof and/or the inhibition of IL-13 mediated inflammation. In one embodiment, the formulations of the invention are sterile and in suitable form for a particular method of administration to a subject with an inflammatory disease, in particular an IL-13-mediated and/or C/CLP-associated disease.

The invention provides methods for preventing, managing, treating or ameliorating an inflammatory disease (in particular an IL-13-mediated and/or C/CLP-associated disease) or one or more symptoms thereof, said method comprising: (a) administering to a subject in need thereof a dose of a prophylactically or therapeutically effective amount of a formulation comprising one or more C/CLP antagonist, and (b) administering one or more subsequent doses of said formulation, to maintain a plasma concentration of the C/CLP antagonist at a desirable level (e.g., about 0.1 to about 100 μg/ml), which continuously blocks a C/CLP activity. In a specific embodiment, the plasma concentration of the C/CLP antagonist is maintained at 10 μg/ml, 15 μg/ml, 20 μg/ml, 25 μg/ml, 30 μg/ml, 35 μg/ml, 40 μg/ml, 45 μg/ml or 50 μg/ml. In a specific embodiment, said effective amount of C/CLP antagonist to be administered is between at least 1 mg/kg and 100 mg/kg per dose. In another specific embodiment, said effective amount of C/CLP antagonist to be administered is between at least 1 mg/kg and 20 mg/kg per dose. In another specific embodiment, said effective amount of C/CLP antagonist to be administered is between at least 4 mg/kg and 10 mg/kg per dose. In yet another specific embodiment, said effective amount of C/CLP antagonist to be administered is between 50 mg and 250 mg per dose. In still another specific embodiment, said effective amount of C/CLP antagonist to be administered is between 100 mg and 200 mg per dose.

### 5.11 Methods of Identifying a Useful Compound

The invention encompasses a method for identifying a compound or intervention that treats an inflammatory disease. One skilled in the art would appreciate, based upon the disclosure provided herein, that assessing the expression and/or activity of a C/CLP can be performed by assessing, among other things, the levels of a C/CLP or the mRNA that encodes it in a cell or tissue, and the like, and then the level can be compared to the level in an otherwise identical cell or tissue to which the compound is not administered. Alternatively, the level of C/CLP or the mRNA that encode it in a cell or tissue contacted with a compound can be compared with the level of the C/CLP or its mRNA in the cell or tissue prior to administration of the compound. One skilled in the art would understand that such compound can be a useful potential therapeutic for treating and/or preventing an inflammatory disease, and for treating and preventing an IL-13 mediated inflammatory disease, and/or for treating a disease associated with and/or mediated by a Th2 inflammatory response.

The skilled artisan would further appreciate that the methods for identifying a compound useful for antagonizing at least one activity of a C/CLP include methods wherein a compound is administered to a cell, tissue, or animal. That is, the skilled artisan, when armed with the present disclosure, would recognize that the teachings herein can be used to identify a compound useful for antagonizing at least one activity of a C/CLP in a cell or tissue expressing a C/CLP. Such cells and tissues are well known in the art, and can include cells and tissues derived from a transgenic non-human animal having altered expression IL-13, IL-4, and/or a chitinase-like molecule, or a transgenic animal comprising an inflammatory disease, and/or a cell or tissue derived therefrom.

Additionally, a cell or tissue comprising expression of a C/CLP can be contacted with a compound and the level of the C/CLP can be assessed and compared to the level of the C/CLP in the cell and/or tissue prior to administration of the compound. Further, the level of the chitinase-like molecule can be compared to the level of the C/CLP in an otherwise identical cell or tissue not contacted with the compound.

The present invention also provides methods for identifying a compound useful for antagonizing at least one activity of a C/CLP wherein the compound is effective when directly utilized on a C/CLP. For example, a compound may be added directly to a solution comprising a C/CLP molecule and incubated for a period of time. The activity of the C/CLP co-incubated with said compound is then compared to the activity of a control C/CLP (e.g., a C/CLP sample which was not co-incubated). This method is particularly useful for the identification of compounds which inhibit enzymatic activity (e.g., chitinase activity) or inhibit C/CLP protein-protein interactions (e.g., galectin binding). These methods may also be used to identify a compound which inhibits an activity of a C/CLP on a cell and/or tissue when the co-incubated C/CLP and control C/CLP are subsequently administered to cells and/or tissues (e.g., YKL-40 mediated stimulation of cytokine release and/or lung inflammation). One skilled in the art will recognize that the C/CLP used in these methods may be purified from an endogenous source or may be from a recombinant source produced using methods well known in the art.

One skilled in the art would appreciate, based upon the disclosure provided herein, that the cell or tissue can express endogenous C/CLP, but the invention further encompasses a cell or tissue that has been modified to express a C/CLP not otherwise expressed in the tissue, e.g., a nucleic encoding a chitinase-like molecule of interest can be introduced and expressed in the cell or tissue where it is not typically expressed, or is expressed at a different level than after the nucleic acid is introduced into the cell or tissue. Thus, the invention includes a wide plethora of assays, comprising a cell, tissue, or an animal, wherein the level of a chitinase-like molecule can be assessed in the presence or absence of a compound. Accordingly, the skilled artisan would be able to identify a compound using the methods disclosed herein and cell culture and cell propagation techniques well known in the art to assess the ability of a compound to affect the level of a C/CLP. Therefore, the present invention further encompasses a method of identifying a compound useful for inhibiting a chitinase-like molecule in a cell or tissue, as well as in an animal.

One of skill in the art would understand, based upon the disclosure provided herein, that the invention includes a method of identifying a compound useful for treating an inflammatory disease in a mammal. As would be understood by one skilled in the art armed with the teachings provided herein, the method encompasses identifying a compound that treats an inflammatory disease in a cell, tissue or subject. The method comprises identifying a substance or compound that antagonizes the expression and/or activity of a C/CLP in a mammal (including in a cell or tissue thereof), preferably in the respiratory tract. This is because, as discussed elsewhere herein, the data demonstrate that inhibiting the expression or activity of a C/CLP provides a therapeutic benefit thereby treating or preventing an inflammatory disease mediated by or associated with increased expression or activity of a C/CLP. This is because the present invention discloses that increased level of a C/CLP is associated with, or mediates, such disease and antagonizing a C/CLP (e.g. AMCase, and the like), using a C/CLP antagonist, such as, but not limited to, an antibody that specifically binds with AMCase prevents and/or treats the disease.

Thus, the skilled artisan, once armed with the teachings of the invention, would appreciate that a compound antagonizing a C/CLP is a powerful potential therapeutic or prophylactic treatment of inflammatory disease, such that identification of such a compound identifies a potential therapeutic for such disease.

### 5.12 Kits

The invention encompasses various kits relating to antagonizing C/CLPs in a mammal which are useful, because, as disclosed elsewhere herein, antagonizing C/CLPs provides a method of treating or preventing inflammatory disease in a mammal. Thus, in one aspect, the invention includes a kit for treating an inflammatory disease in a mammal. The kit comprises an effective amount of a C/CLP antagonist. The kit further comprises an applicator and an instructional material for the use thereof to be used in accordance with the teachings provided herein.

The invention includes various kits which comprise a compound, such as a an antibody that specifically binds a C/CLP, as well as a nucleic acid encoding such an antibody, a nucleic acid complementary to a nucleic acid encoding a C/CLP but in an antisense orientation with respect to transcription, a ribozyme capable of cleaving a single-stranded chitinase-like molecule RNA, an applicator, and instructional materials which describe use of the compound to perform the methods of the invention.

Although exemplary kits are described below, the contents of other useful kits will be apparent to the skilled artisan in light of the present disclosure. Each of these kits is included within the invention.

In one aspect, the invention includes kits for treating or preventing an inflammatory disease and an inflammatory disease mediated by IL-13. The kit is used pursuant to the methods disclosed in the invention. Briefly, the kit may be used to contact a mammal with a chemical compound that inhibits C/CLP, or a nucleic acid complementary to a nucleic acid encoding a C/CLP where the nucleic acid is in an antisense orientation with respect to transcription to reduce expression of a C/CLP, or with an antibody that specifically binds with a C/CLP or a nucleic acid encoding the antibody, wherein the decreased expression, amount, or activity of a C/CLP mediates an beneficial effect in the mammal. Moreover, the kit comprises an applicator and an instructional material for the use of the kit. These instructions simply embody the examples provided herein.

The kit includes a pharmaceutically-acceptable carrier. The composition is provided in an appropriate amount as set forth elsewhere herein. Further, the route of administration and the frequency of administration are as previously set forth elsewhere herein.

### 6. EXAMPLES

The invention is now described with reference to the following examples. These examples are provided for the purpose of illustration only and the invention should in no way be construed as being limited to these examples but rather should be construed to encompass any and all variations which become evident as a result of the teachings provided herein.

### 6.1 Example 1

### Expression and Characterization of C/CLPs

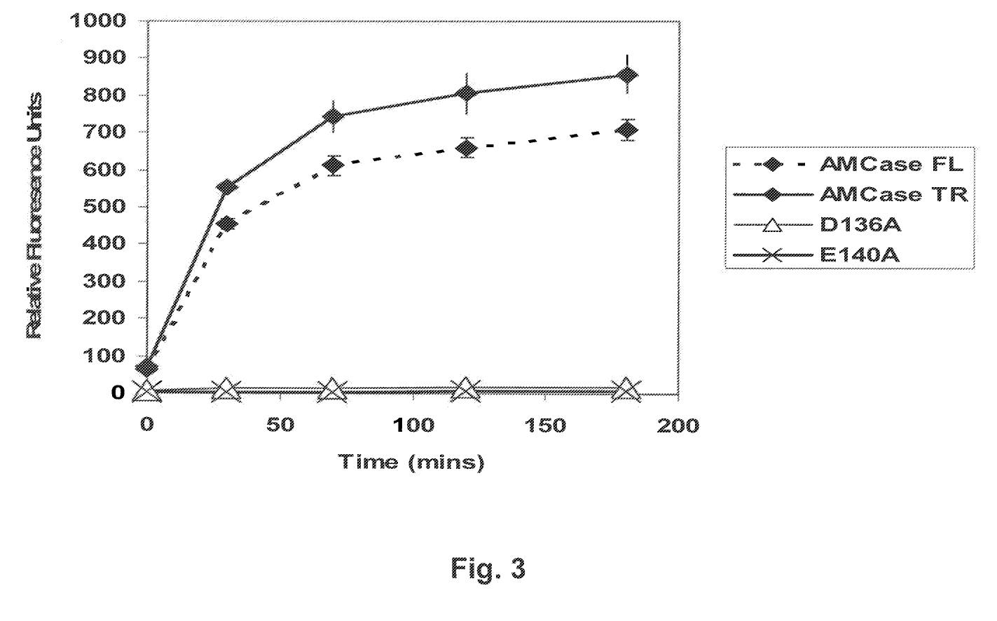

Multiple C/CLPs were expressed as recombinant proteins, including several point mutations and a truncation of AMCase. The activity of these proteins was assayed. It was found that full length AMCase and a truncated form lacking the C-terminal carbohydrate domain, were active in a 4-MU assay (soluble substrate). We found that the truncated form routinely exhibited higher activity (FIG. 3). Two single point mutants were generated, D136A and E140A. These amino acids are highly conserved among the “true” chitinases. Substitution of either position completely abolished activity (FIG. 3).

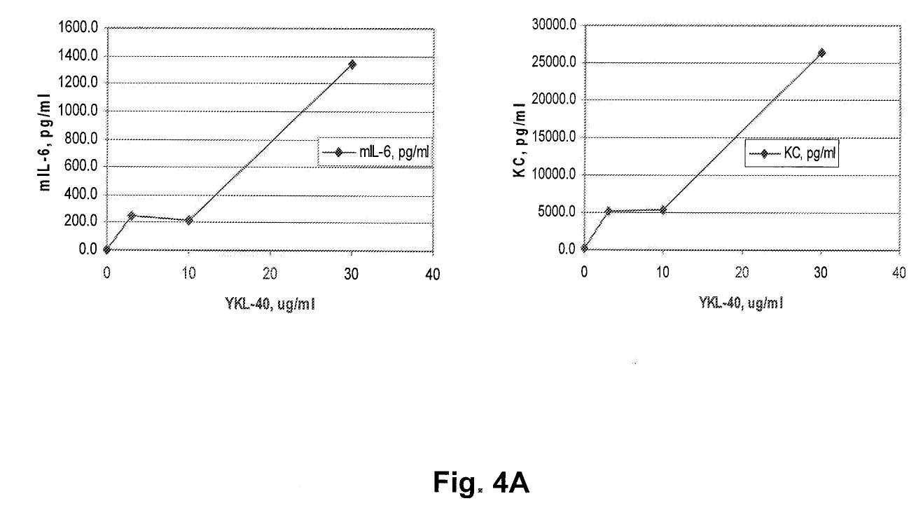

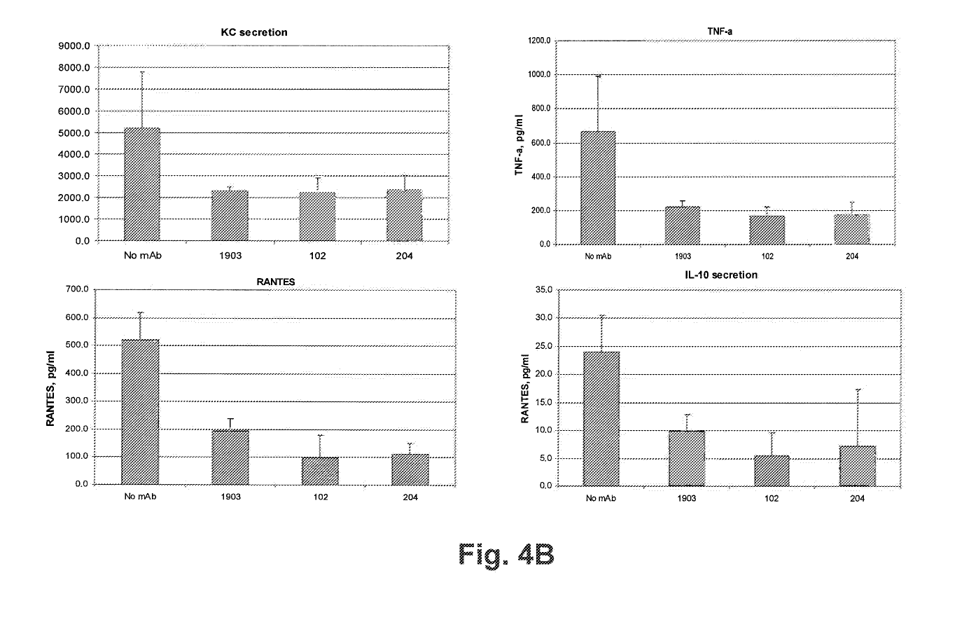

Recombinant mouse YKL-40/BRP-39 (also referred to simply as “mYKL-40”) was tested for its ability to modulate a variety of chemokines and cytokines from peritoneal macrophages. mYLK-40 treatment resulted in an increase in the levels of both IL-6 and KC in cell supernates. The levels of IL-6 and KC in response to treatment with increasing concentrations of mYKL-40 are plotted in FIG. 4 (left and right panels respectively. Additional experiments demonstrated that treatment of peritoneal macrophages with mYKL-40 also induced IL-10, RANTES and TNF-α (data not shown and FIG. 4B). FIG. 4B demonstrates that several monoclonal anti-mYKL-40 antibodies (1903, 102 and 204, described below) inhibited the mYLK-40 stimulated release of KC (top left); TNFα (top right); RANTES (bottom left); and IL-10 (bottom right). The antibodies reduced the levels of KC in the supernate by ˜56% while the levels of TNF-α, RANTES and IL-10 were reduced by ˜70-75%.

### Materials and Methods

Cloning of C/CLPs for expression in mammalian systems: The construction of histidine tagged versions of Ym1,Ym2, mouse and human AMCase, mouse and human chitotriosidase, mouse and human YKL40 (BRP39), human TSA1902L and human YKL39 was accomplished by PCR from cDNA libraries (mouse or human), a forward primer with a convenient restriction site, a Kozak consensus sequence (underlined) upstream of the initiation codon, and a reverse primer that inserted 6 histidine codons (underlined) between the last naturally occurring codon and the stop codon (italics), followed by a convenient restriction site (see Table 5 for full description of several primers). For non-his-tagged versions primers lacking the 6 histidine codons were utilized. After amplification, the PCR products were digested with restriction endonucleases and ligated into pcDNA3.1. The sequence of the clone was confirmed by DNA sequence analysis.

TABLE 5
Primers used for cloning and construction of polynucleotides encoding C/CLPs

| Name of gene | Forward primer | Reverse primer |
|---|---|---|
| Ym1 | 5′GGATCCCAACATGGCCAAGCTCATTC | 5′GAATT [symbol] TAATGGTGATGGTGATGGTGA |
| | TTGTC3′ | TAAGGGCCCTTGCAACT3′ |
| | (SEQ ID NO: 31) | (SEQ ID NO: 32) |
| Ym2 | 5′GGATCCCAACATGGCCAAGCTCATTC | 5′GAATT [symbol] TAGTGATGGTGATGGTGATGA |
| | TTGTC3′ | AGCTCCCCTCGATAAGAGGC3′ |
| | (SEQ ID NO: 33) | (SEQ ID NO: 34) |
| Mouse AMCase | 5′GGATCCCACCATGGCCAAGCTACTTC | 5′GAATT [symbol] TAGTGATGGTGATGGTGATGT |
| | TCGTC3′ | GGCCAGTTGCAGCAATT3′ |
| | (SEQ ID NO: 35) | (SEQ ID NO: 36) |
| Mouse | 5′AGATCTCACCATGGTGCAGTCCCTGG | 5′GAATT [symbol] TAGTGATGGTGATGGTGATGG |
| chitotriosidase | CCTGG3′ | ACTGGAGTTGGATGGGG3′ |
| | (SEQ ID NO: 37) | (SEQ ID NO: 38) |
| Mouse YKL-40 | 5′GGATCCACCATGGGCATGAGGGCGGC | 5′TCTAGACTAGTGATGGTGATGGTGATG |
| | ACTG3′ | AGCCAGGGCATCCTTGAT3′ |
| | (SEQ ID NO: 39) | (SEQ ID NO: 40) |
| Human AMCase | 5′GGATCCCACCATGACAAAGCTTATTC | 5′GAATTCTTAGTGATGGTGATGGTGATG |
| | TCCTCAC3′ | TGCCCAGTTGCAGCAATC3′ |
| | (SEQ ID NO: 41) | (SEQ ID NO: 42) |
| human | 5′AAGCTTCACCATGGTGCGGTCTGTGG | 5′GGATC [symbol] TAGTGATGGTGATGGTGATGA |
| chitotriosidase | CCTGGGC3′ | TTCCAGGTGCAGCATTTG3′ |
| | (SEQ ID NO: 43) | (SEQ ID NO: 44) |
| humanYKL-40 | 5′AAGCTTCACCATGGGTGTGAAGGCGT | 5′GGATC [symbol] TAATGGTGATGGTGATGGTGC |
| | CTCAA3′ | GTTGCAGCGAGTGCATC3′ |
| | (SEQ ID NO: 45) | (SEQ ID NO: 46) |
| human | 5′AAGGATCCACCATGGTTTCTACTCCT | 5′GAATTCTTAGTGATGGTGATGGTGATG |
| TSA1902L | GAGAACCG3′ | TGCCCAGTTGCAGCAATC3′ |
| | (SEQ ID NO: 47) | (SEQ ID NO: 48) |
| humanYKL39 | 5′AAGCTTCACCATGGGAGCAACCACCA | 5′GGATC [symbol] TAATGGTGATGGTGATGGTGC |
| | TGGAC3′ | GTTGCAGCGAGTGCATC3′ |
| | (SEQ ID NO: 49) | (SEQ ID NO: 50) |

Forward primer: restriction endonuclease site-bolded, Kozak consensus-underlined.
Reverse primer: restriction endonuclease site-bolded, stop codon-italics, 6 histidine tag-underlined.

Expression of C/CLPs in mammalian cells: HEK293 cells are transformed using the FreeSyle 293 Expression System for large scale transfections (InVitrogen, Carlsbad, Calif.). Briefly, 450 micrograms of plasmid is mixed with 200 microliters of 293fectin diluted in a final volume of 30 ml Optimem. This is added to a 450 ml culture of 293 cells growing in 293 FreeStyle Media. The culture supernatant is harvested 3 days after transfection, and the cells are fed. A second harvest is done after an additional 3 days. The His-tagged protein is then purified by applying the culture supernatant to a Nickel NTA sepharose column. The protein is eluted with an Imidazole gradient.

Activity Assays Used: Chitinase activity was assayed using two different but commonly used assays. One assay (4-Mu-Chitotrioside assay) utilized 4-methylumbelliferyl-β-D-N,N′,N″-triacetylchitotrise as a substrate (see, e.g., Hollak et al., 1994, J. Clin. Invest. 93: 1288-92). The other (chitin-azure assay) utilized chitin azure as a substrate (see, e.g., Thompson et al., (2001) Appl Environ Microbiol. 67:4001-4008).

Isolation of peritoneal macrophages: 4-6 week old Balb/c mice were injected i.p. with 1 ml of fluid thioglyclollate medium (Becton, Dickinson and Company, Sparks, Md.). 4 days later peritoneal macrophages were harvested by lavage (ice-cold PBS w/o Ca, Mg, 10 ml/mouse).Cells were washed with PBS twice and resuspended in 10% FBS/RPMI 1640 medium (Glutamax, 25 mM Hepes, 1× antibiotic-antimycotic (Invitrogen, Grand Island, N.Y.) at 2.5×10⁶ cells/ml. Cells were plated in 96 well TC plates (100 μl/well). After overnight incubation at 37° C. nonadherent cells were removed by washing with serum free RPMI 1640. 100 μl/well of 10% FBS/RPMI1640 were added and cells were cultured for 72 hr.

Cytokine Induction Assays: isolated peritoneal macrophages (described above) were washed with PBS, proteins and antibodies, concentrated supernatants, were added in 2% FBS/RPMI 1640, 100 μl/well. After 18 hr incubation supernatants were collected and cytokine concentrations were measured using mouse cytokine multiplex kit (Upstate, Lake Placid, N.Y.) on Luminex 100.

### 6.2 Example 2

### Generation and Characterization of Anti-C/CLP Antibodies

A number of antibodies including polyclonal, and both rodent and fully human monoclonal antibodies were generated which recognize mouse and/or human AMCase. In addition, polyclonal and/or rodent monoclonal antibodies were generated which recognize mouse and/or human ChT, YKL-40 and mouse YM-1 and YM-2. The rabbit polyclonal antibodies and rodent monoclonal antibodies were generated using standards methods. The fully human monoclonal antibodies were isolated from a commercial phage display by panning and converted into full length IgGs. The binding characteristics of several monoclonal and polyclonal antibodies are detailed in Table 2. The variable domains of several of the isolated fully human monoclonals are shown in FIG. 6A-D. The panel of antibodies was tested for both cross reactivity and competition. Several of the antibodies (e.g., 4F8 and 149.159) were found to cross react with other C/CLPs this is not unexpected given the high degree of homology between the molecules. Other antibodies were highly specific (e.g., 128.148 and Z4). Interestingly several antibodies (e.g., 171.204, Z8 and 317.171) did not bind to a truncated version of AMCase lacking the C-terminal chitin binding domain (CBD). These antibodies may indeed bind the CBD, or bind to an epitope that is destroyed by the absence of the CBD (Table 2).

Competition analysis revealed that the antibodies analyzed thus far bind to at least five distinct sites on mouse AMCase, and four distinct sites on human AMCase (FIG. 7).

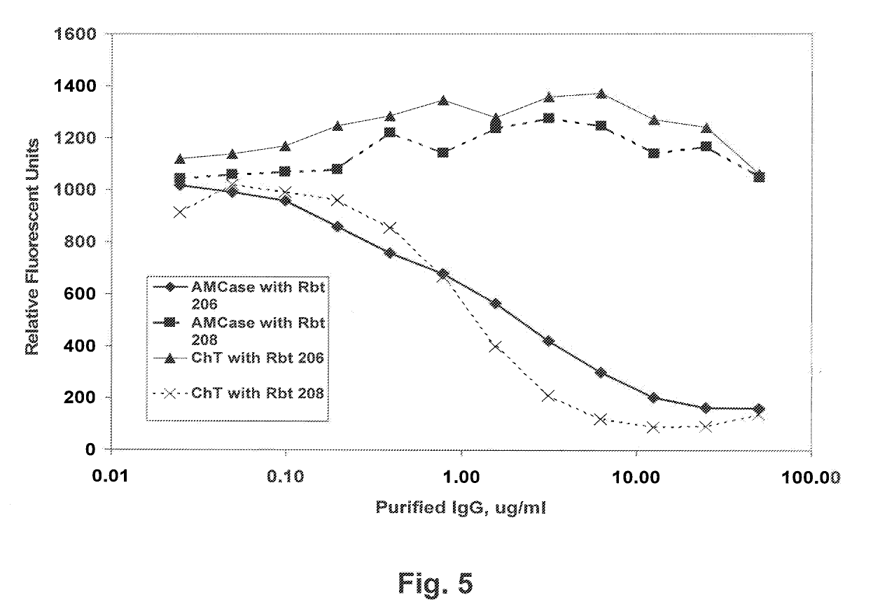

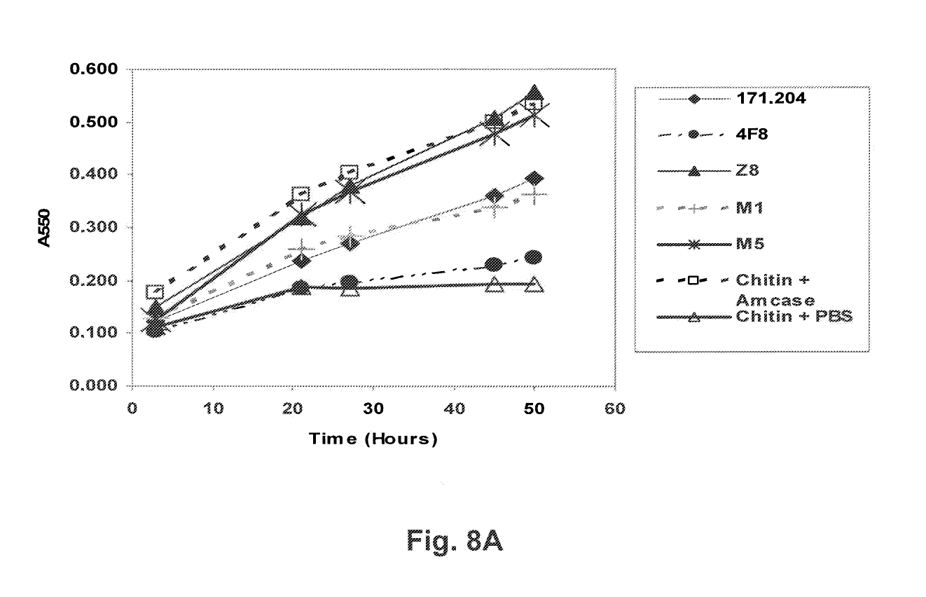

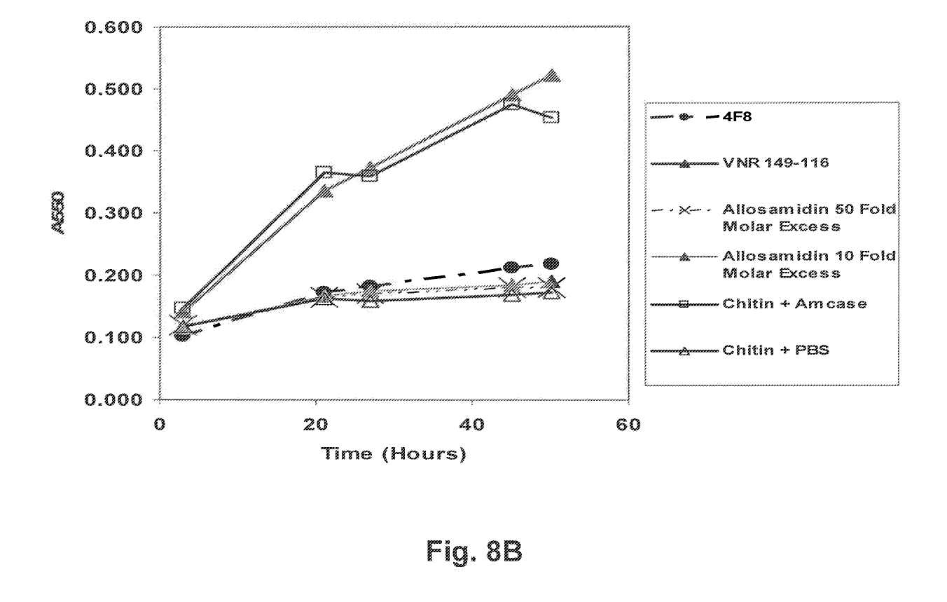

Several antibodies were assayed for their ability to in antagonize/inhibit the chitinase activity of either AMCase or ChT. FIG. 5 shows the results of inhibition studies performed on two rabbit polyclonal sera's using the 4-Mu-Chitotrioside assay. Rabbit 206 was immunized with AMCase while rabbit 208 was immunized with ChT. The results indicate that antisera from rabbit 206 specifically inhibits AMCase chitinase activity while antisera from rabbit 208 specifically inhibits ChT chitinase activity. FIG. 8 shows the results of inhibit studies performed on several monoclonal antibodies using the chitin-azure assay. Several antibodies (Z8 and M5) did not appear to significantly inhibit the chitinase activity of AMCase. Two other antibodies (171.204 and M1) appeared to modestly inhibit the chitinase activity of AMCase. However, one monoclonal antibody, 4F8 (AMCase specific) was seen to potently inhibit the chitinase activity of AMCase (FIG. 8A). Additional studies of this antibody show that it is as potent an inhibitor as allosamidin (FIG. 8B).

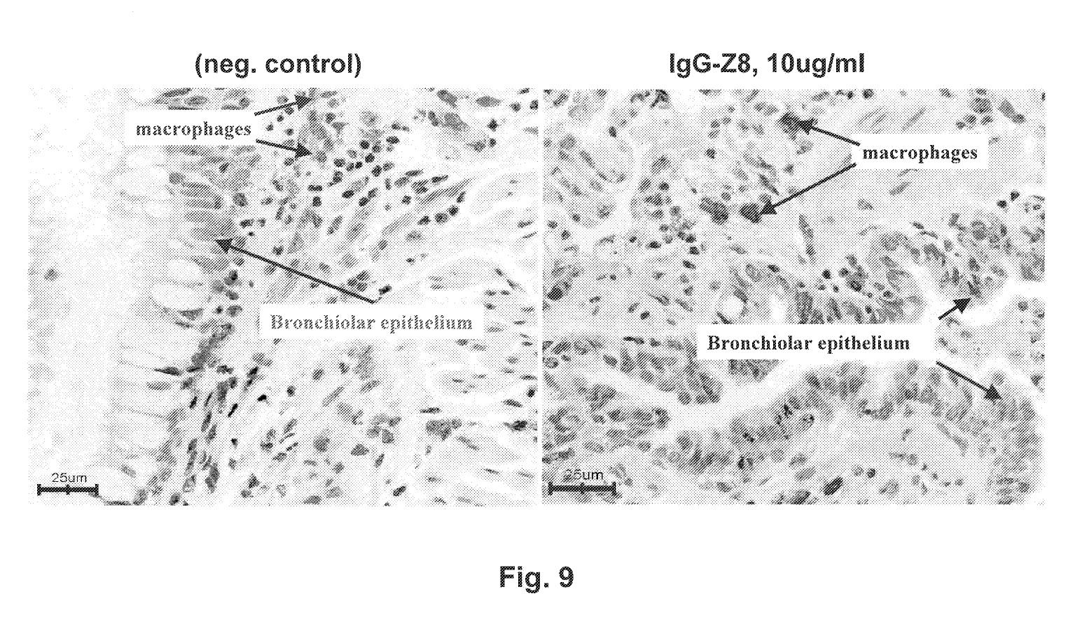

Several antibodies were tested for their ability to stain AMCase in tissue. FIG. 9 shows that the human monoclonal, Z8, stains AMCase present in the macrophages of a human asthmatic lung.

### Materials and Methods

Polyclonal Antibodies: Polyclonal antibodies were generated against a number of C/CLP molecules, in particular full length YM1 (including antibody 2870), mouse and human AMCase (including antibodies 206 and 655, respectively), mouse and human YKL-40 (including antibodies 212 and 204, respectively) and mouse and human ChT (including antibodies 208 and 657, respectively), as well as certain AMCase peptides from the chitin binding and catalytic domains, using standard protocols (see, e.g., Harlow, E., and Lane, D., 1988, Antibodies: A laboratory Manual, Cold Spring Harbor Laboratory, Cold Spring Harbor, N.Y.). The binding characteristics of several polyclonal sera are detailed in Table 2.

Rodent Monoclonal Antibodies: Monoclonal antibodies were generated against a number of C/CLP molecules, in particular mAMCase and hAMCase, using standard protocols (see, e.g., Harlow, E., and Lane, D., 1988, Antibodies: A laboratory Manual, Cold Spring Harbor Laboratory, Cold Spring Harbor, N.Y.). Specifically, antibody AMC182-2S.171.204 (abbreviated as “171.204”) was generated as follows: SJL mice (Jackson Laboratories) were immunized with 10 μg/mouse human AMCase in GERBU adjuvant in both metatarsals on Days 1, 7, 12 and 14. Mice were bled from the retro-orbital sinus on Day 10 to determine serum titers. On day 15, spleen and draining lymph nodes were removed for fusion and the tissues were fused separately with NS-0 Cells and plated in ClonaCell HY Medium (StemCell Technologies). Supernatants were screened by ELISA on plates coated with Human and Murine AMCase at 0.5 μg/ml in PBS. Clone AMC182-2S.171.204 cross-reacts equally well with human and murine AMCase. Limiting Dilution Cloning in 96 well plates was performed to isolate and stabilize the cell line. Antibody AMC183-2.128.148 (abbreviated as “128.148”) was generated as follows: Wistar Rats (Harlan) were immunized with 20 μg/rat ECF-L AF in TiterMax Gold, i.m. on Days 1 and boosted with 20 μg/rat murine AMCase on Day 28. Rats were bled from the retro-orbital sinus on Day 35 to determine serum titers. Spleens were harvest 3 days after an i.p. boost with 20 μg/rat human and murine AMCase in PBS and were fused with NS-0 cells. Fused cells were plated in ClonaCell HY Medium (StemCell Technologies). Supernatants were screened by ELISA on plates coated with Human and Murine AMCase at 0.5 μg/ml in PBS. Positive clones were categorized as wither Human Specific, Murine Specific or Human Murine cross-reactive. All positive clones were also tested by ELISA for inhibition by labeled AMC182-25.171.204. Clone AMC183-2.128.148 was not inhibited in the ELISA indicating that it reacted with a different epitope. Limiting dilution cloning in 96 well plates was performed to isolated and stabilize the cell line. Antibody 4F8.C3.B6.G12 (abbreviated as “4F8”) was generated as follows: Armenian Hamsters (Cytogen Research and Development, Inc.) were immunized with 20 μg murine AMCase in TiterMax Gold, i.m. on Days 1 and 28. Hamsters were bled from the retro-orbital sinus on Day 35 to determine serum titers. Spleens were harvest 3 days after an i.p. boost with 20 μg/hamster murine AMCase in PBS and were fused.

Hybridomas generated from mice/rats/hamsters immunized with recombinant C/CLP molecules were screened for C/CLP specific antibodies using an enzyme-linked immunosorbent assay (ELISA). Antibody preparations from positive clones were further tested for cross reactivity to other C/CLPs (see Table 2). In addition to the clones described in Table 2, several anti-mouseYKL-40 monoclonal antibodies were generated including 1903; 102 and 204.

Human Monoclonal Antibodies: Human monoclonal antibodies were isolated from a commercially available phage display library expressing the variable region of human antibodies, by panning against immobilized full length C/CLPs or immobilized C/CLP peptides. The heavy and light chain variable regions were sequenced (see FIG. 6A-D). The isolated clones were then used to generate full length IgG antibodies and the resulting antibodies were tested for cross reactivity as described above (see Table 2).

BIAcore Analysis: Competition studies were performed, using standard BIAcore methods, to determine which antibodies have shared, overlapping or distinct binding sites (FIG. 7).

Inhibition Studies: Several antibodies were assayed for their ability to in antagonize/inhibit the chitinase activity of either AMCase or ChT using either the 4-Mu-Chitotrioside assay or the Chitin-azure assay described above.

### 6.3 Example 3

### Asthma Studies

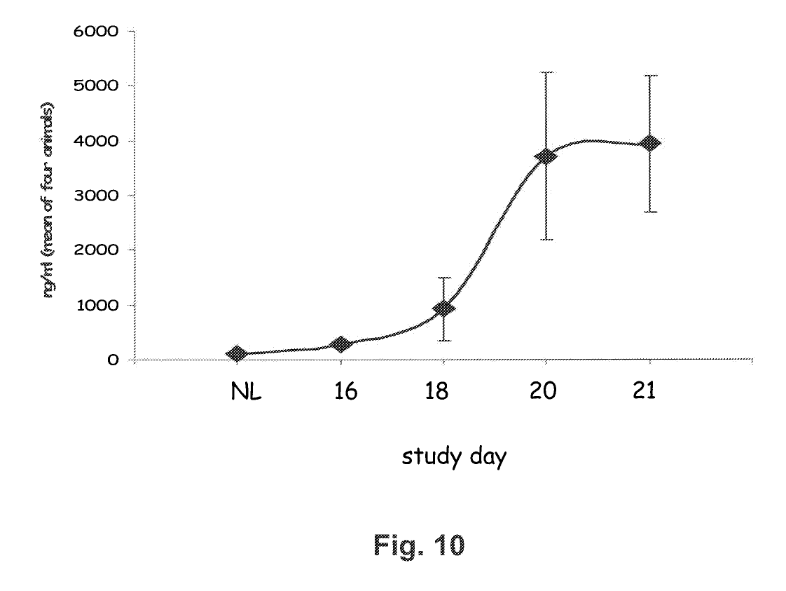

Previous studies (Zhu et al., 2004, Science 304:1678-82) indicated that AMCase was upregulated in the lungs of allergen induced asthmatic mice. To determine the contribution of each “true” chitinase to the increase in activity seen OVA sensitized mice were subsequently challenged on days 16, 18 and 20. Tissues were harvested 4 hours post challenge and assayed for chitinase activity using the 4-Mu-Chitotrioside assay. A significant increase in chitinase activity was detected after the second challenge at day 18 which continued to increase with subsequent challenges (FIG. 10). To determine which chitinase was responsible for the increase in activity the lung homogenates were also assayed in the presence of the inhibitory antibodies 206 or 208. As shown in FIG. 11, the anti-AMCase antibody (206) inhibited the chitinase activity present in the homogenates by an average of 85%. The anti-ChT antibody did not significantly inhibit the chitinase activity. These data confirm, for the first time, that the increase in chitinase activity seen in the lung of allergen-induced asthma in this model are due to the increase in the levels of AMCase.

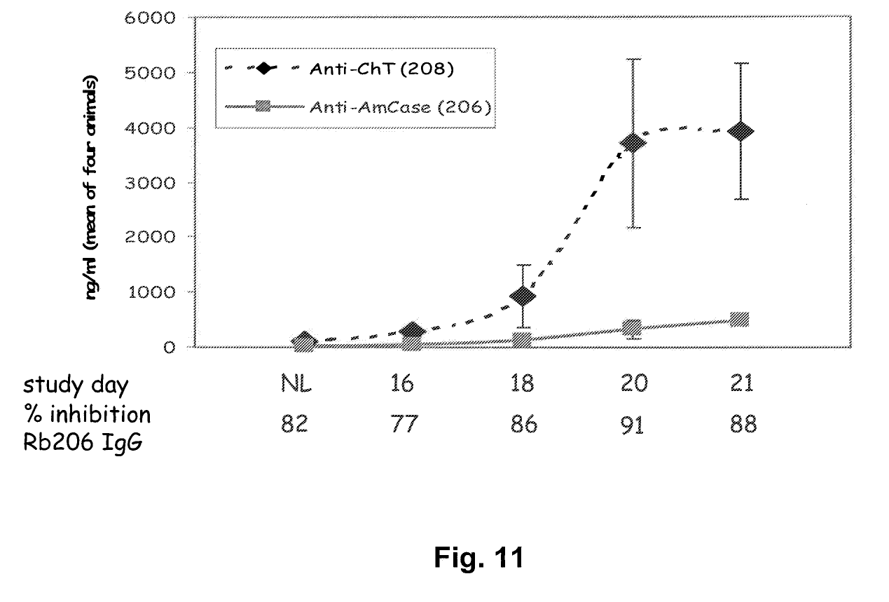

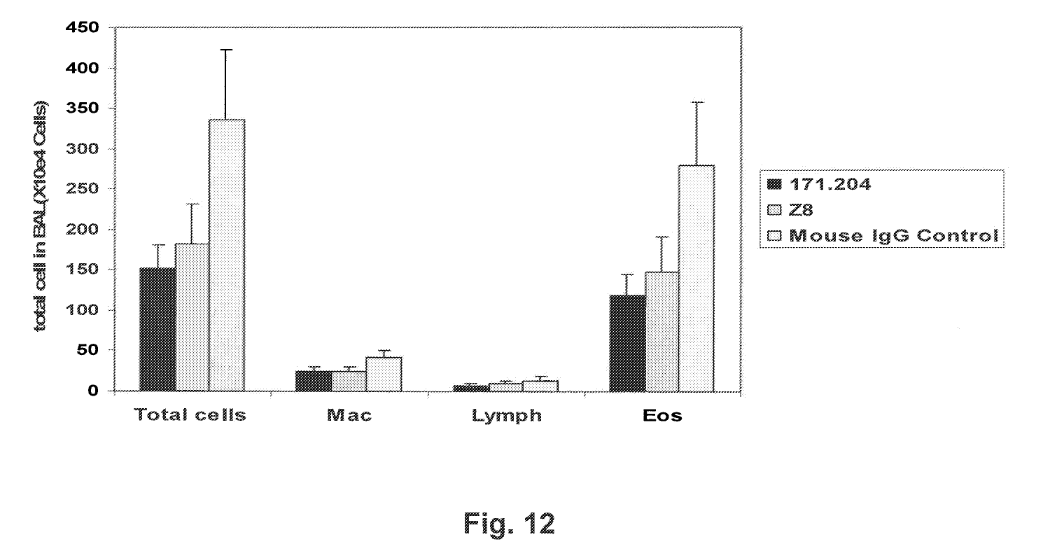

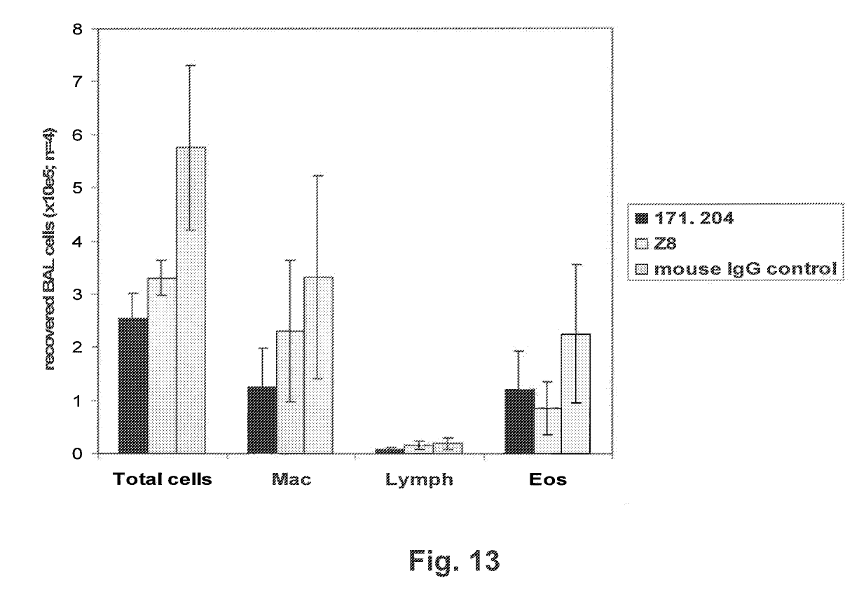

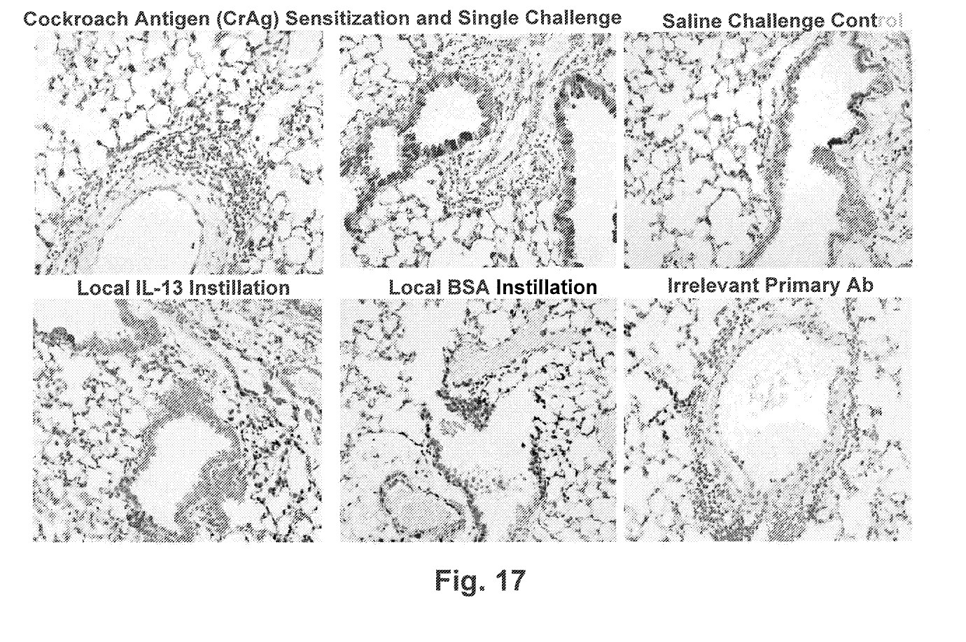

The ability of several antibodies to inhibit the cellular infiltration associated with allergen-induced asthma was tested using two different antigens. FIG. 12 shows the results using the same antigen (ovalbumin) used in previous studies (see, Zhu et al. supra). From this study, it is clear that two antibodies, 171.204 and Z8 inhibit cellular infiltration relative to an antibody isotype control (1A7). 171.204 and Z8 were further tested in another allergen-induced asthma model using the chitin containing cockroach antigen (CrAg). Both antibodies were again shown to inhibit cellular infiltration as compared to a control antibody (FIG. 13). In both studies 171.204 was seen to be slightly more efficacious then Z8. Interestingly, both 171.204 and Z8 do not bind to the truncated form of AMCase while 4F8 does. This may indicate that the CBD (missing from the truncated form of AMCase) plays a critical role in mediating cellular infiltration in allergen-induced asthma. Immunohistochemical detection of AMCase in lungs of CrAg sensitized mice did not show much AMCase immunoreactivity (FIG. 17 top left). However, staining of the alveolar macrophages and bronchiolar epithelium was apparent after challenge with CrAg but not after a saline challenge control (FIG. 17 top middle and right, respectively). RT-PCR of lung tissue from CrAg treated and mock treated mice show a dramatic increase in AMCase expression levels in the CrAg treated mice (data not shown).

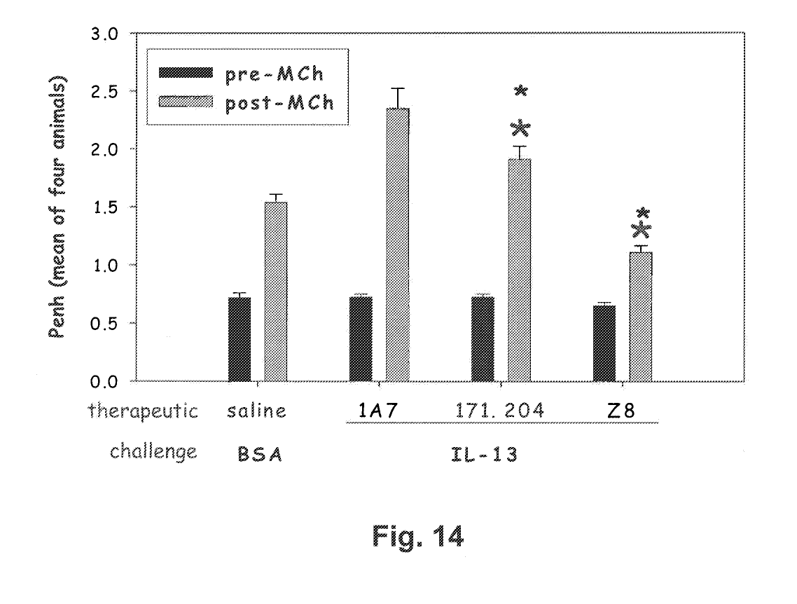

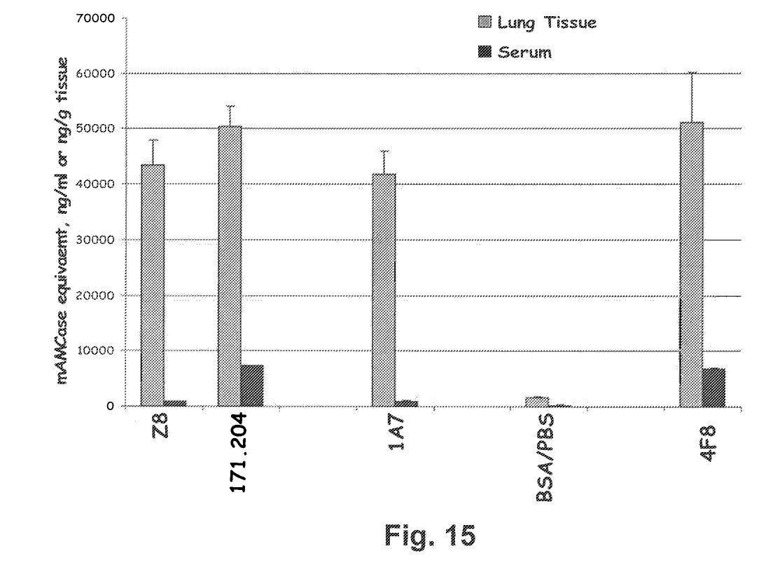

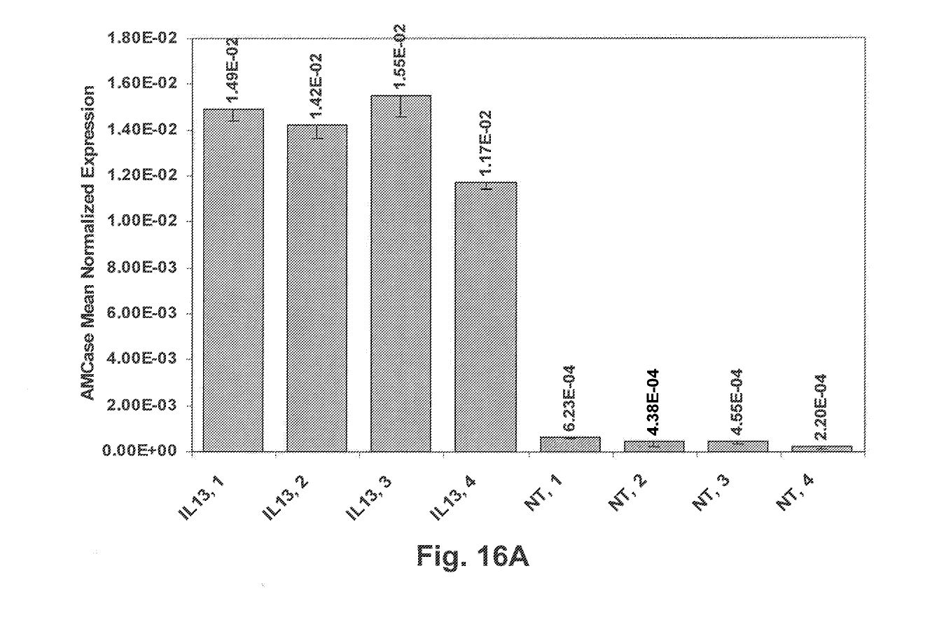

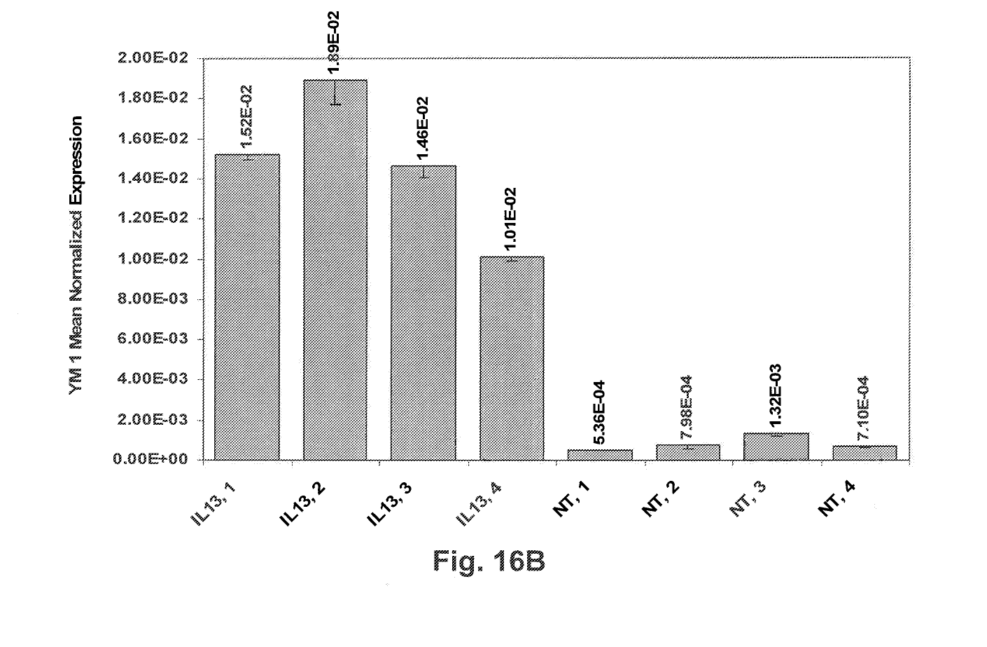

Direct instillation of IL-13 into the lung is seen to enhance methacholine induced airway hyperresponsiveness (AHR). Real-time PCR of lung tissue demonstrate that both AMCase and the related chitinase-like protein, YM1 are upregulated about one to two logs in mice treated with IL-13 (FIGS. 16A and B, respectively). In addition, immunohistochemical detection of AMCase in lungs of IL-13 treated mice revealed AMCase immunoreactivity in alveolar macrophages and bronchiolar epithelium but not in control mice treated with local instillation of BSA (FIG. 17 bottom left and middle panels, respectively). Penh scores were measured pre and post methacholine challenge in mice treated with saline alone or IL-13 in combination with either 171.204, Z8 or an isotype control antibody (1A7). Both 171.204 and Z8 antibodies also showed efficacy in reducing IL-13 enhanced AHR as compared to the antibody isotype control (FIG. 14). Neither 171.204 nor Z8 reduce the amount of chitinolytic activity recovered from either lung tissue or serum after IL-13 treatment (FIG. 15).

IL-13 treatment of primary mouse alveolar macrophages resulted in AMCase mRNA induction, measured by quantitative real-time PCR. In contrast no AMCase mRNA expression was observed in other mouse primary cells and immortalized cell lines in response to IL-13 or other stimuli causing alternative activation (parasite antigens)(data not shown). AMCase expression in various human primary cells and cell lines after IL-13 stimulation was also examined. No AMCase mRNA was observed in monolayer cultures of bronchiolar cell lines and primary tracheal epithelial cells grown at air-liquid interface. In contrast to AMCase, the chitinase-like protein YKL-40 was expressed in air-liquid interface cultures (data not shown).

### Materials and Methods

Allergen-induced Asthma in Mice: For the time course of chitinase expression mice (Balb/c) were sensitized on day 1 and again on day 8 followed by challenge with 2.5 μg of ovalbumin delivered intranasally on day 16, 18 and 20. Tissue was harvested 4 hrs post challenge and the study was terminated at day 21.

The antibody treatment studies performed using the ovalbumin model of allergen induced asthma were performed essentially as described in Zhou et al. (supra). Antibodies were administered at a dose of 0.5 ml i.p. every other day starting one day before the challenge. A total of four mice were in each group.

The antibody treatment studies performed using the cockroach model of allergen induced asthma were performed as follows, mice (Balb/c) were sensitized on day 1 and again on day 8 followed by challenge with 2.5 μg of cockroach antigen delivered intranasally on day 16. On day 15 (prior to challenge) and day 17.5 (post challenge) the animals were treated with 10 mg/kg of antibody i.p. Tissue was harvested after challenge, prior to administration of the second dose of antibody and post administration of the second dose of antibody. The study was terminated on day 19.

Airway Hyperresponsiveness (AHR) Measurements: AHR is measured in unrestrained conscious mice by using barometric plethysmography (Buxco Electronics, CT). AHR is expressed as the fold increase in Penh (enhanced pause). Mice are placed in whole-body plethysmographic chambers and exposed for ˜1 min to aerosolized saline and subsequently to increasing concentrations of aerosolized Mch (1-300 mg/ml). Recordings are taken for ˜5 min after each aerosolization. The Penh values measured during each 5 min cycle are averaged and expressed for each Mch concentration as a percentage of the baseline Penh value following saline exposure.

IL-13 enhanced AHR in Mice: 8 week old BALB/C mice were treated 3-4 times with 1-5 μg/animal IL-13 by endotracheal intubation at roughly 24 hour intervals. Mice were challenged with either BSA or methacholine. For the treatment groups the mice were treated with either saline, or ˜10 mg/kg of antibody (anti-AMCase antibodies or an isotype control antibody) i.p. prior to IL-13 treatment. Penh scores pre and post methacholine treatment were measured essentially as described above. For protein analysis, 24 hours after last treatment, mice are euthanized and bronchoalveolar lavage, lung tissue and serum are collected and analyzed for AMCase expression, AMCase protein concentration and chitinolytic activity.

Mouse Lung Homogenates: Lungs were removed from mice, flushed with PBS, blotted gently on paper towel. One half of each lung (˜150 mg) was placed in 2 ml cryotube and frozen at −80° C. 750 μl PBS and a steel bead was added to each tube with frozen tissue and samples were homogenized on TissueLyser (Qiagen). 2×2 minute pulses at 30 vibrations per second were used.

RT-PCR: Mouse lung tissue (stored in RNAlater) and cell cultures were homogenized in RLT+2-ME and RNA was purified using Qiagen's Rneasy Miniprep Kit. Real time PCR was performed using ABI Tagman gene expression primers/probe and the ABI Prism 7000 SDS instrument.

Immunohistochemistry: tissues are deparaffinized and rehydrated as needed. Endogenous peroxidase activity may be blocked.

For mouse tissues: AMCase staining—biotinylated purified anti-mAMCase Rabbit IgG (Medi Rabbit #206) at 1 μg/ml, preblocked with 5 m/ml of BRP-39, YM1, and YM2 to suppress crossreactivity. Gal-3 staining—biotinylated affinity purified goat anti-mGal-3 IgG (RnD Systems # BAF1197) at 1 μg/ml. Gal-4 staining—biotinylated affinity purified goat anti-mGal-4 IgG (RnD Systems #BAF2128) at 1 μg/ml. Detection with streptavidin HRP or other detection agent followed by Mayer's Hematoxylin counter staining.

For human tissues: AMCase staining—purified anti-hAMCase rabbit IgG (Medi, Rbt 655), at for 10 μg/ml, preblocked with 1 or 10 μg/ml of chitotriosidase (ChTRase) to suppress crossreactivity. ChTRase staining—purified anti-hChTRase Rbt IgG (Medi, Rbt 657), at for 10 μg/ml, preblocked with 1 or 10 μg/ml of AMCase to suppress crossreactivity. In all instances, the primary antibody was applied for 2 hrs. at room temperature. 0.5 μg/mL of anti-goat secondary antibody was used for the human tissues, and 0.5 μg/mL of Streptavidin-HRP for all the assays.

### 6.4 Example 4

AMCase Interacts with Galectins

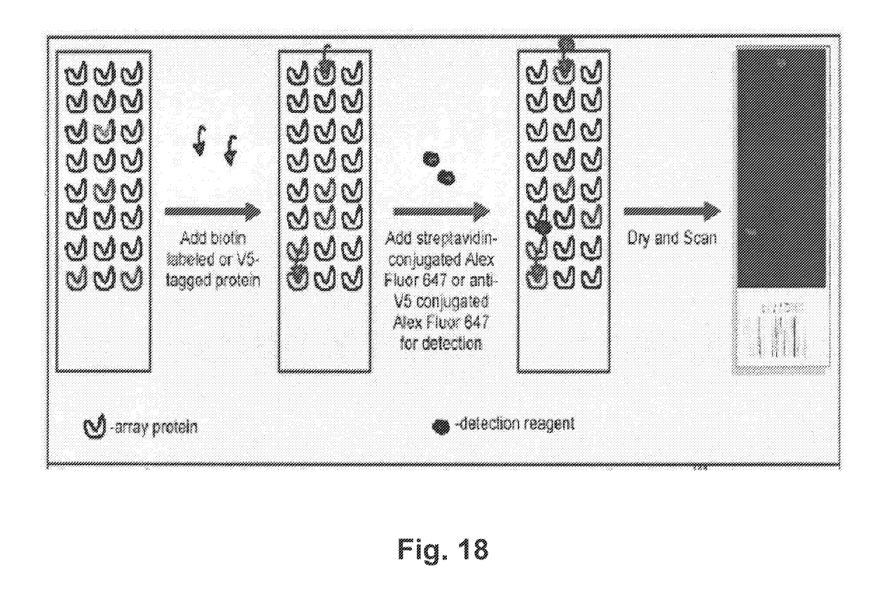

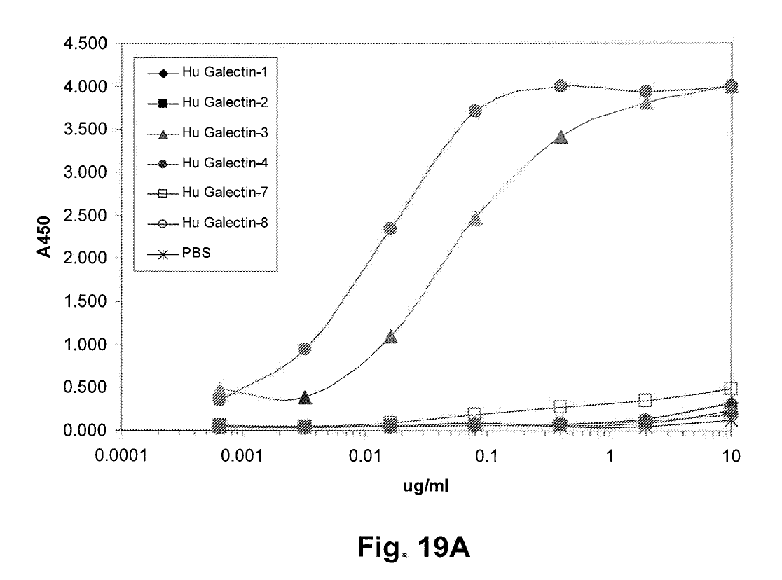

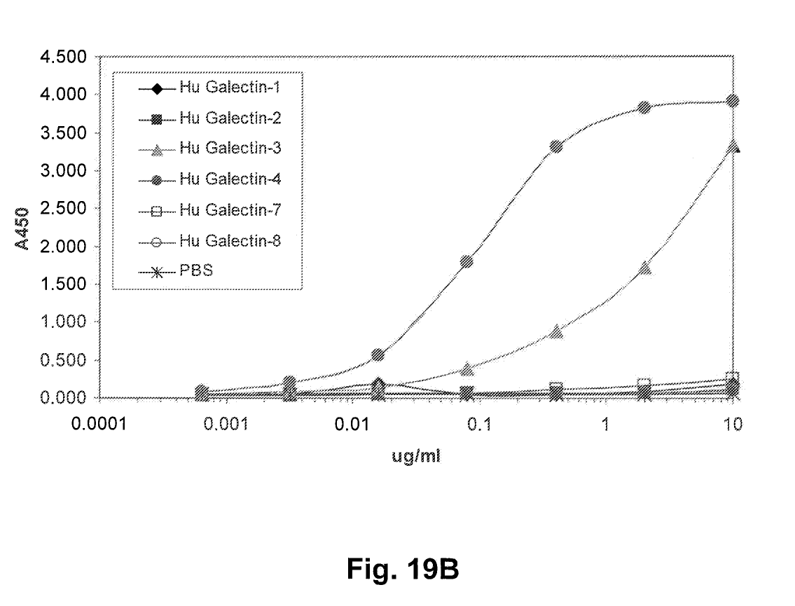

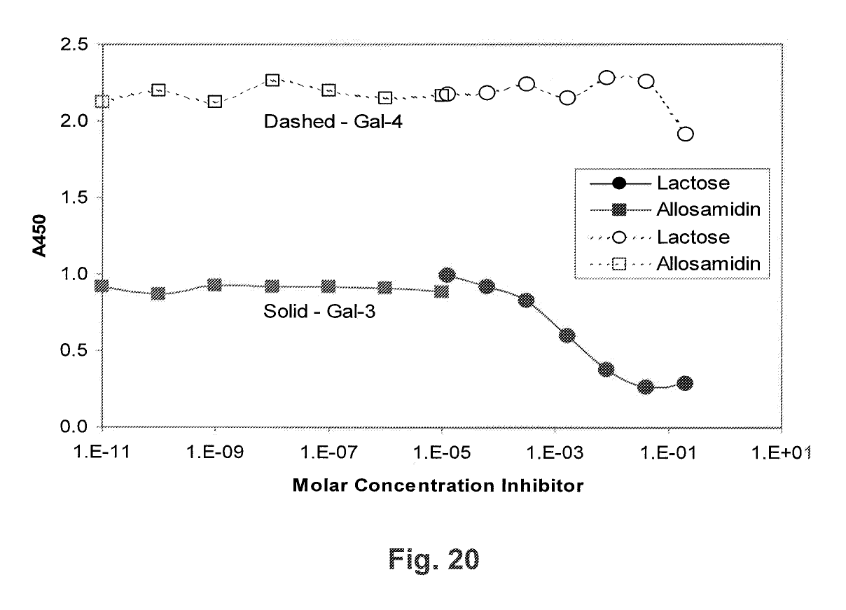

Protein-Protein interaction array profiling was performed by a commercial vendor (Invitrogen™) using labeled AMCase and a protein array. The basic scheme used is shown in FIG. 18. Several proteins were identified including galectin-3 (LGALS3) and galectin-8 (LGALS8). The interaction of both full length and C-terminally truncated human AMCase lacking the chitin binding domain with human galectin-3 and human galectin-4 was confirmed by ELISA (FIGS. 19A and 19B, respectively). Similarly, mouse AMCase was also found to bind mouse galectin-3 and mouse galectin-4 (data not shown). This interaction can be inhibited by the anti-mAMCase antibodies 171.204 and Z8 (data not shown). The binding of AMCase to galectin-3 and galectin-4 can be inhibited by lactose but not allosamidin (FIG. 20). This data suggests that the carbohydrate recognition domain (CRD) domain is involved in AMCase binding.

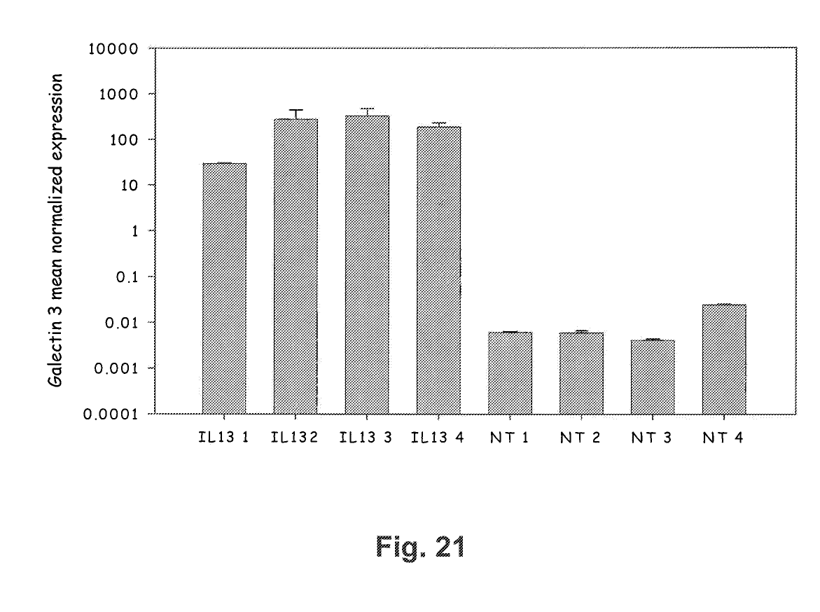

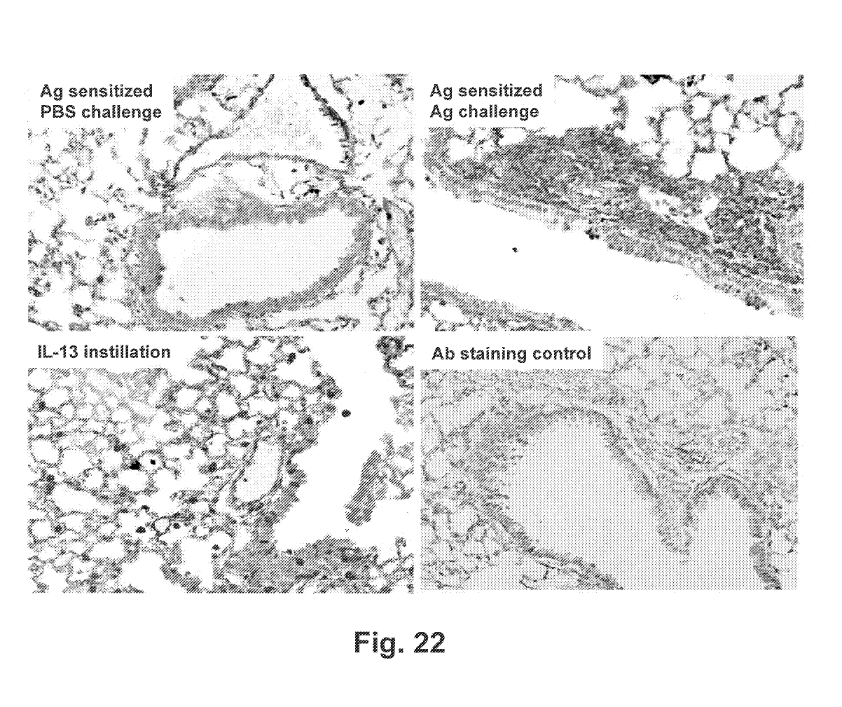

Galectin-3 expression levels were examined in several asthma models. RT-PCR analysis of lung tissue from IL-13 treated mice shows that galectin-3 levels increase by about 3 to 4 logs relative to PBS control treated mice (FIG. 21). By immunohistochemistry staining, galectin-3 is seen in lungs of mice sensitized with CrAg followed by a single challenge (FIG. 22, top right) but not in mice challenged with saline (FIG. 22, top left). Similarly, galectin-3 staining was seen in the lungs of IL-13 treated mice (FIG. 22, bottom left) but not in mice treated with local instillation of BSA (not shown).

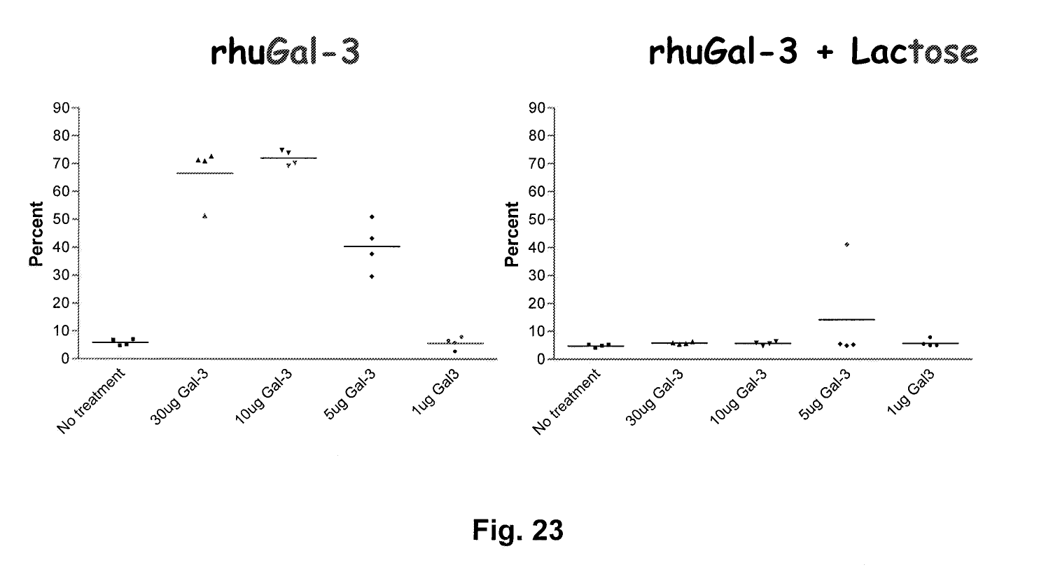

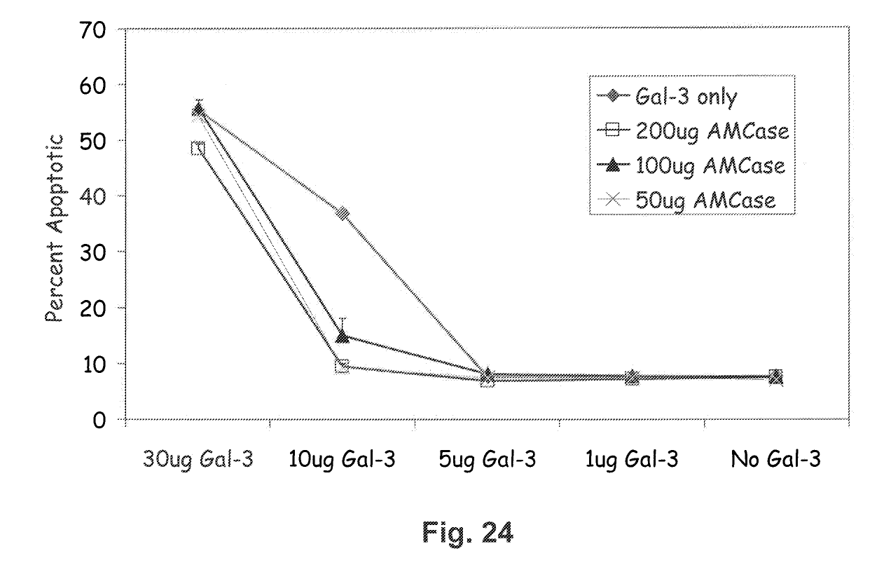

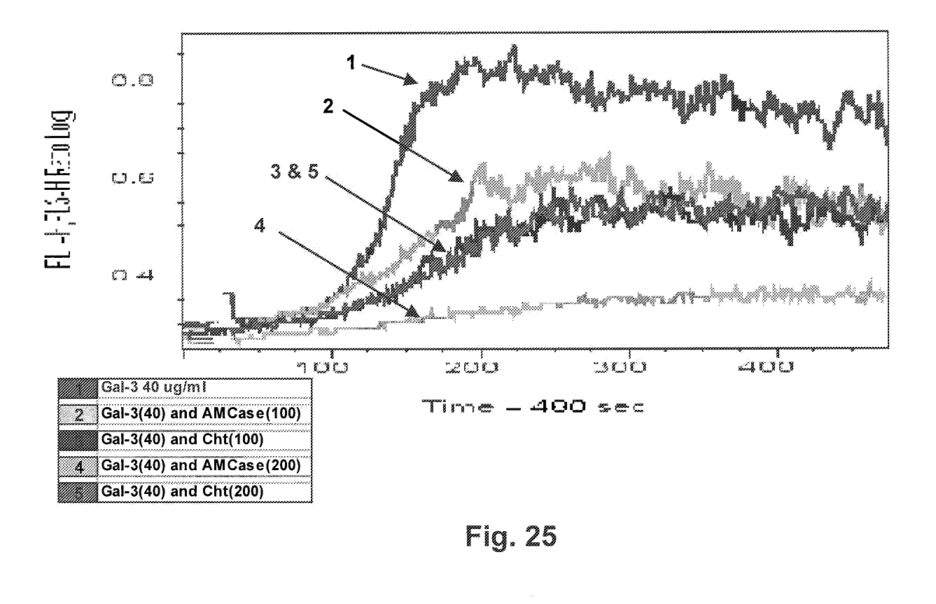

Galectin-3 was shown to induce apoptosis in Jurkat T cells in a dose dependent manor (FIG. 23, left). This activity was abolished by the addition of lactose (FIG. 23, right). Similarly, galectin-3 was shown to induced apoptosis in memory T cells isolated from human blood. This activity could also be inhibited by AMCase (FIG. 24). Galectin-3 was also shown to mediate calcium flux in Jurkat T cells (FIG. 25, line 1) and this flux could be inhibited by the addition of either AMCase (FIG. 25 lines 2 and 4) or Chitotriosidase (FIG. 25 lines 3 and 5). These data suggest that AMCase may block galectin-induced apoptosis and prolong inflammation in asthma. Accordingly, agents which interfere with the binding of AMCase to galectin may reduce inflammation.

### Materials and Methods

C/CLP-Galectin Interaction ELISA: Galectin is immobilized on a microtiter plate in DPBS over night at 2-8° C. The plates are then washed with DPBS, 0.1% Tween 20 and blocked for 1 hour at room temperature. Serial dilutions of biotin labeled AMCase are added to each well and incubated for ˜1 hour at room temperature followed by HRP labeled streptavidin. Horseradish peroxydase activity is detected with TMB substrate (KPL, MD) and the reaction is quenched with 1% H2SO4. The absorbance is read at 450 nm.

IL-13 enhanced AHR and allergen induced asthma in Mice; Immunohistochemistrv; Tissue homogenates and RT-PCR: were all performed essentially as described above.

Galectin Induced Calcium Flux: Jurkat cells were labeled with Fluo-4AM and Fura-Red. galectin-3 (40 m/ml) was added to the cells and Ca flux was analyzed as FL1/FL3. Analysis was performed for 5 minutes. For interaction analysis gal-3 (40 m/ml) is pre-incubated with huAMCase (100-200 μg/ml) or huChTrase (100-200 μg/ml) for 15 mins at RT prior to addition.

### 6.5 Example 5

### Chitinases are Present in Human Asthmatic Lung Samples

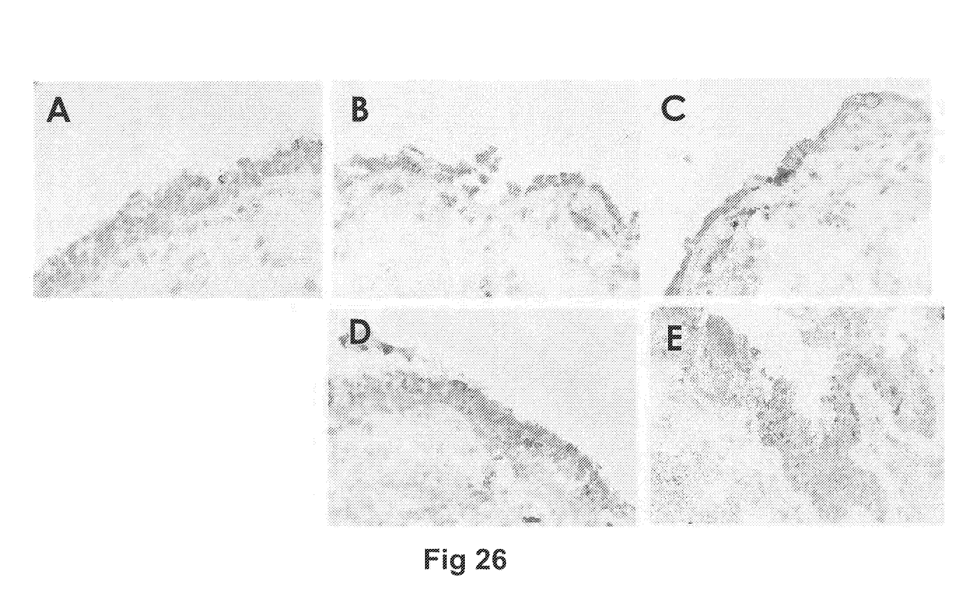

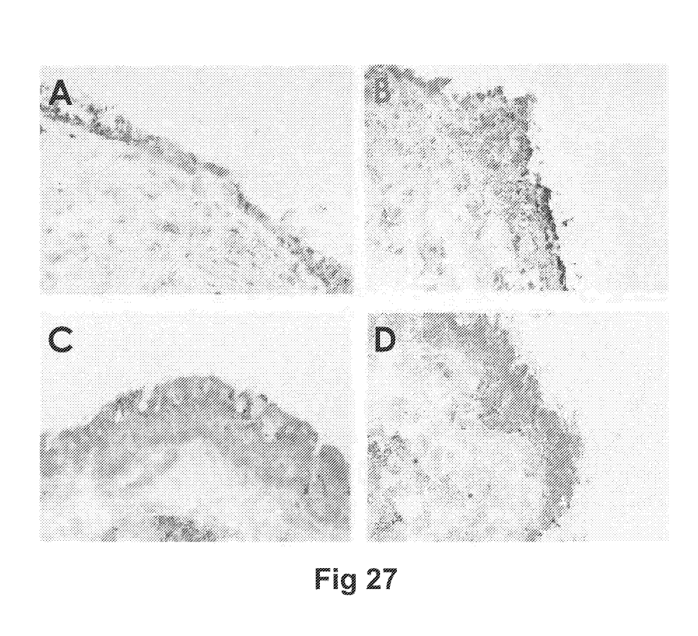

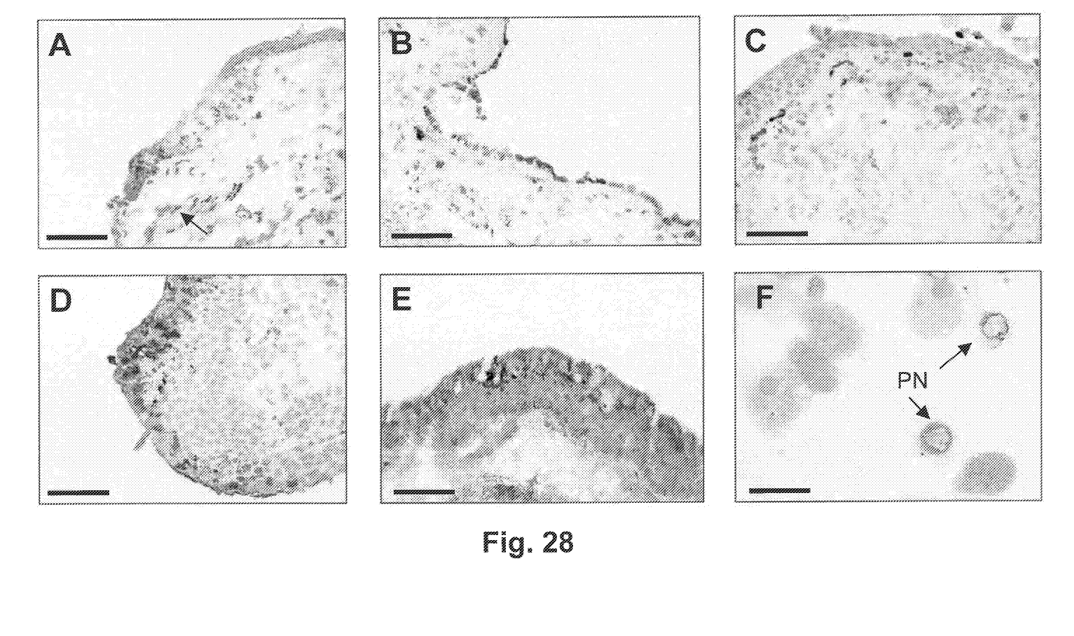

Previous studies have reported that AMCase is upregulated in the epithelial cells and macrophages in the lung tissue of samples taken from patients with asthma (Zhu, Z., et al., 2004, Science, 304: 1678-82). Here we confirm and expand those studies by immunostaining for AMCase and other C/CLPs in samples from control and asthmatic individuals. Immunostaining of bronchial biopsies from control and asthmatic individuals demonstrated that cells which stain positive for C/CLPs, specifically AMCase (FIG. 26), ChT (FIG. 27) and YKL-40 (FIG. 28), are scarce in normal individuals while numerous positively staining cells are seen in asthmatics. For each C/CLP examined positively staining monocyte/macrophages accumulated in subepithelial areas of mild, intermittent and moderate asthmatics (FIGS. 26B-D; 27B; and 28B-C). Bronchial tissue sections from severe asthmatics had numerous positively staining monocyte/macrophages infiltrating both the bronchial epithelium and submucosa (FIGS. 26E; 27C-D; and 28D). YKL-40 localized in the cytoplasm of macrophages and neutrophils, as confirmed on representative cytospin preparations of cells collected by bronchial lavage from severe asthmatics (FIG. 28F).

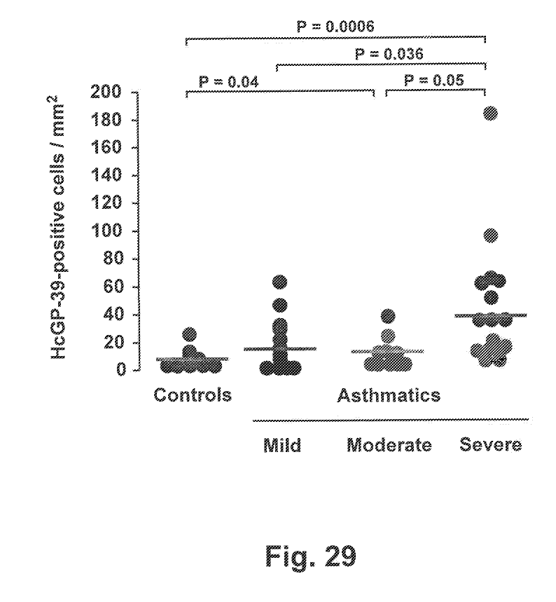

Cell count analysis showed a few but detectable YKL40-positive cells in bronchial biopsies from controls (median [interquartile range], 3.1 per mm² [2.1-7.4]). These numbers were higher in mild and moderate asthmatics (13.7 per mm² [3.0-28.3] and 11.3 [7.9-17.5], p=0.102 and p=0.04 versus controls, respectively). In severe asthmatics the number of YKL-40-positive cells was further augmented (23.1 per mm² [15.2-54.1]) and it was statistically different as compared to the other patient groups (FIG. 29). These data correlate well with an increase in the serum levels of YLK-40 seen in severe asthmatics (data not shown).

### Materials and Methods

Fiberoptic bronchoscopy and sample collection: The bronchoscopy was performed by the same operator, according to the guidelines outlined by the American Thoracic Society (1), as described (2). The subjects were premedicated with 200 μg inhaled salbutamol and the nose and larynx were anesthetized with 2 and 0.5% xylocain spray, respectively. A bronchial lavage was performed with 50 ml of sterile saline, and 4 biopsy specimens were taken from the subcarinae of the right middle lobe (B4-B5) and the lower lobe (troncus basalis bifurcations: B7-B8, B8-B9, B9-B10). A standard type biopsy forceps and an OLYMPUS bronchoscope (model BF P20, Olympus Optical, Tokyo, Japan) were used. Bronchial biopsies were immediately put on cork disks, covered by Tissue-Tek compound (Sakura Finetek, Zoeterwoude, The Netherlands) and snap-frozen in liquid nitrogen. The frozen blocks were kept at −80° C. until the assessment of immunohistochemistry, as described below. Bronchial lavages were immediately centrifuged (150 g, 10 min, 4° C.) and 0.5 ml-aliquots were stored at −80° C. Cytospin preparations from cell pellets were fixed in acetone during 10 min at room temperature and stored at −20° until use.

Immunohistochemistry: Serial 5 μm sections of bronchial biopsies collected from the same patients were cut in a cryostat (Leica CM3000, Leica, Rueil-Malmaison, France), collected on glass slides previously coated with γ-methacryloxypropyltrimethoxysilane (Sigma, St. Quentin Fallavier, France) and fixed in acetone for 10 min at room temperature. YKL-40 Immunohistochemistry was performed on bronchial tissue sections and on cytospin preparations using 1 μg/ml of a rabbit antibody directed against human YLK-40 (polyclonal antibody 204), as described (1985. Summary and recommendations of a workshop on the investigative use of fiberoptic bronchoscopy and bronchoalveolar lavage in asthmatics. Am Rev Respir Dis 132:180-182). An anti-rabbit antibody conjugated to biotin and the avidine-alkaline phosphatase complex from the Vectastain kit (Vector, Burlingame, Calif.) were next used, followed by Fast Red staining (DakoCytomation, Trappes, France) and light nuclear Mayer's hematoxylin counterstaining The specificity of the immunostaining was determined using the corresponding isotype, i.e., rabbit IgG (DakoCytomation), instead of the primary antibody. Although the anti-YKL-40 antibody shows minimal crossreactivity with other chitinases in Elisa-based assays, its specificity was further assessed by co-incubating it with 1 μg/ml recombinant human AMCase and ChT for 15 min before adding to slides. Staining for AMCase and ChT was performed essentially as described above. Frozen lung tissues from control healthy volunteers, mild (intermittent) asthma patients, moderate asthma patients, and severe asthma patients, were immunostained with polyclonal rabbit antisera directed against human AMCase (polyclonal antibody 655) or ChT (polyclonal antibody 657). To ensure high specificity of AMCase detection, the AMCase polyclonal antibody was preincubated with recombinant YKL-40 and ChTrase to bind up any antibodies that might be cross reactive; similarly, ChTrase directed polyclonal antibodies were preincubated with recombinant AMCase and YKL-40. Bound antibody was subsequently detected with species-specific, enzymatically labeled secondary antibody and substrate according to standard methods.

### 6.6 Example 6

### Anti-C/CLP Antibodies Inhibit IL-13 Induced Airway Hyperresponsiveness (AHR).

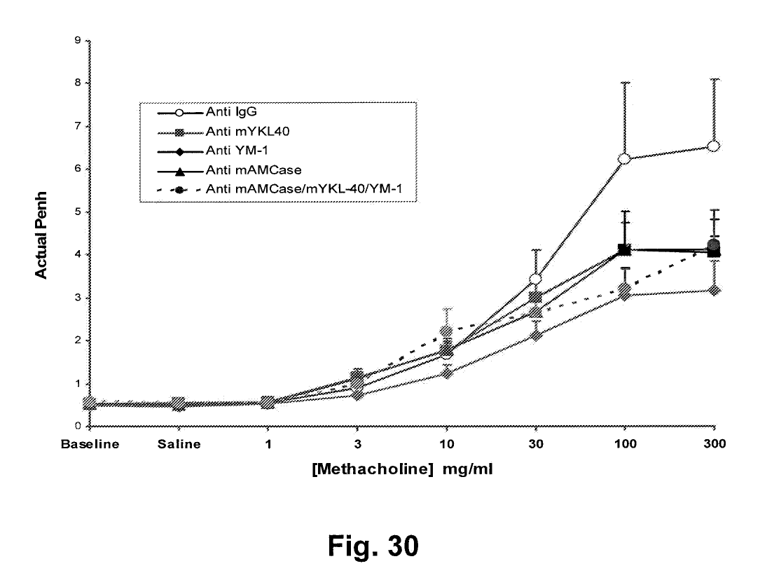

As demonstrated above and in previous studies (Zhu et al., 2004, Science 304:1678-82) administration of anti-mAMCase antibodies inhibits the cellular infiltration and airway hyperresponsiveness associated with allergen- and/or IL-13-induced asthma. In this study we determined that in addition to anti-mAMCase antibodies, anti-mYKL-40 and anti-YM-1 antibodies can inhibit IL-13-induced AHR. FIG. 30 shows the PenH scores for mice pre-treated with a control IgG, anti-mYKL-40, anti-YM1, anti-mAMCase or a combination of anti-mAMCase/mYLK-40/YM-1 antibodies. Each anti-C/CLP antibody was seen to reduce the penh scores by about 40% to 56% (from ˜6.5 to ˜3.2 to 4.2) relative to the control IgG at the highest methacholine challenge dose of 300 mg/ml (FIG. 30). Reductions in penh values were seen for methacholine challenge doses as low as 30 mg/ml. However, pretreatment with a cocktail of all three antibodies did not show any additional decrease in penh score in these studies (FIG. 30).

### Materials and Methods

Pre-treatment and IL-13 Induced Asthma: Day 1 BALB/c mice were injected intraperitoneally with 1.5 mg/mouse or 60 mg/Kg of the following polyclonal antibodies (control IgG , mYKL-40/BRP-39 (antibody 212), AMCase (antibody 206), YM1 (antibody 2870) individually or a mixture of all three antibodies (anti BRP-39/AMCase/YM1)). Two hours later, mice were anesthetized using Isoflurane and administered intratracheally with recombinant murine IL-13 or control low endotoxin BSA (2.5 m/mouse, in 50 μl volume). Mice were given mIL-13 again on Days 2 and 3. Twenty-four hours later, Day 4, mice were assessed for changes in airway hyperresponsiveness essentially as described above (see Example 3). Following AHR measurements, mice were euthanized via CO2 asphyxiation and bled for serum via cardiac puncture. Bronchoalveolar lavage was performed and lung tissue taken for histological and biochemical analysis.

### 6.7 Example 7

### Local Administration of C/CLPs By Adenovector Expression

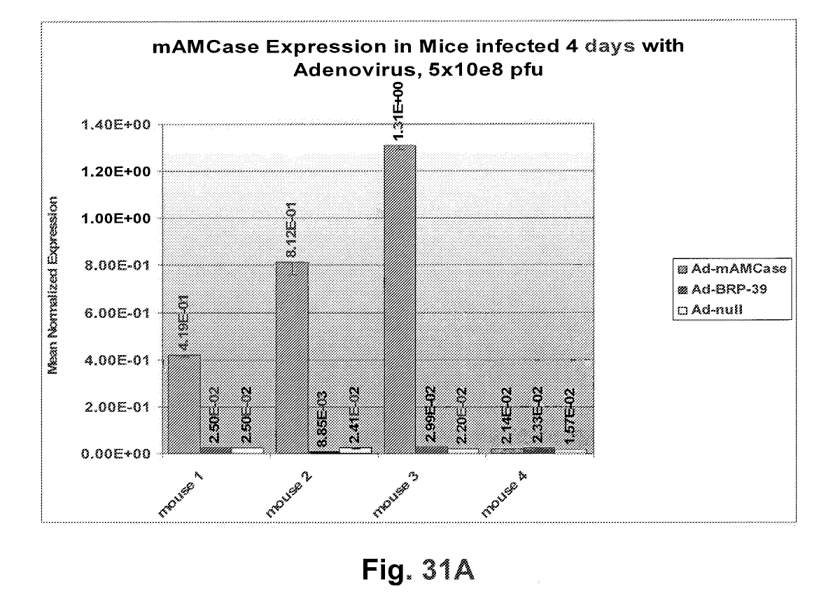

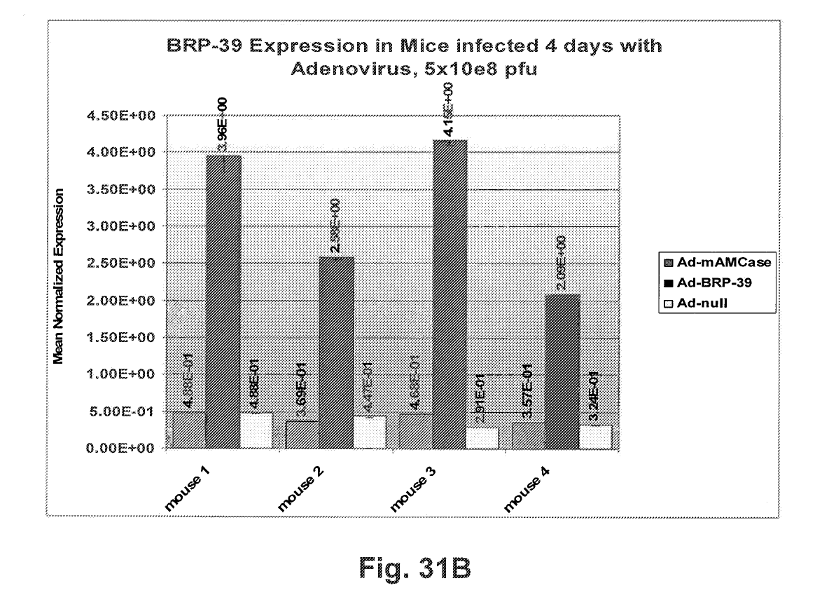

Recombinant adenovectors expressing either mYLK-40/BRP-39, AMCase or a null vector were administered directly into the lungs of 4 mice per treatment group. On day 5 the relative expression levels of mAMCase and mYKL-40/BRP-30 was determined by RT-PCR for each animal in each group (FIG. 31). As shown in FIG. 31A the expression level mAMCase increased by 4 to 13 fold in 3 out of 4 mice infected with mouse AMCase expressing adenovirus. Neither the Null nor mYLK-40/BRP-39 infected mice showed any increase in the relative expression of mAMCase. Similarly, the relative expression level mYKL-40/BRP-39 increased by 2 to 4 fold only in those mice infected with mYKL-40/BRP-39 expressing adenovirus (FIG. 31B).

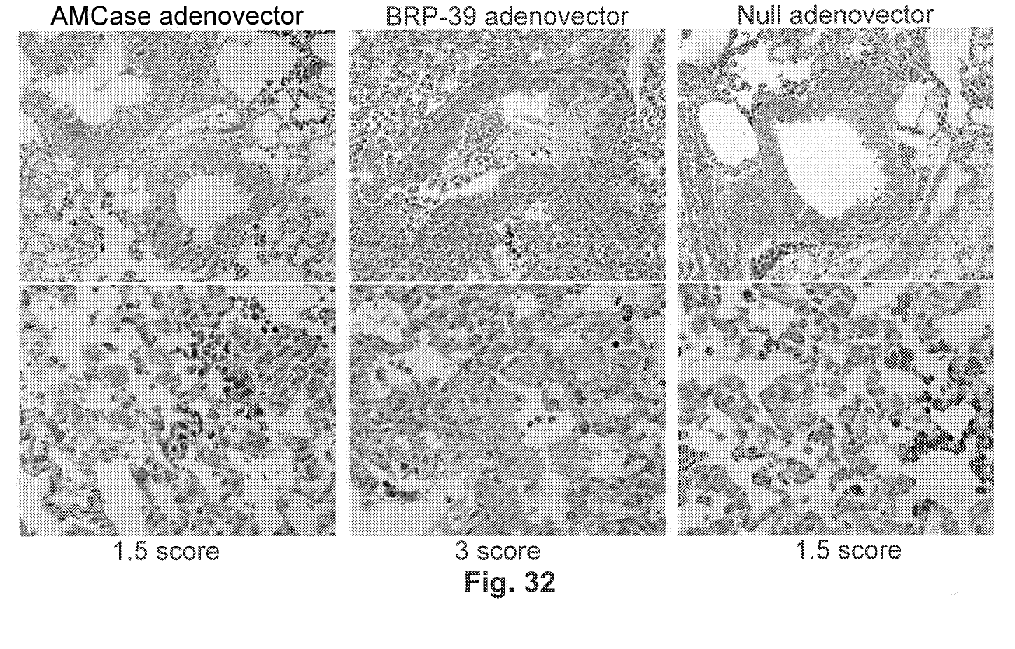

Histological analysis on lung tissue taken after 5 days of treatment shows that animals treated with either the Null adenovector (FIG. 32, right panels) or the AMCase expressing adenovector (FIG. 32, left panels) have mild subacute intra-alveolar mononuclear infiltrate accompanied by occasional neutrophils while tissue from 2 of the animals treated with mYKL-40/BRP-39 expressing adenovector (FIG. 32, middle panels) show diffuse alveolitis plus acute to chronic respiratory bronchiolitis with occlusion (serum plus coagulated protein) that extends to larger bronchioles. The remaining two animals demonstrate intra-alveolar infiltrate similar to but somewhat stronger than null and AMCase adenovector groups (data not shown).

### Materials and Methods

Generation of Ad-mYKL-40/BRP-39 and Ad-mAMCase Adenovectors: CLONING: Plasmid pCDNAmYKL40His was digested with BamHI/SacI and the 839 by fragment containing mYKL-40/BRP-39 was gel purified. The 3′ region of the gene was amplified using sense (5′-tgggagctccaatctcag-3′, SEQ ID NO.: 51) and antisense (5′-aaaagcttctaagccagggcatccttgat-3′, SEQ ID NO.: 52) primers, the PCR product digested with SacI/Hind III and gel purified. The two fragments were then cloned within the BglII/HindIII sites of the pDC316 plasmid. Plasmid pCImAMC was digested with EcoRI/Not I and the 1.5 Kb fragment containing murine AMCase was gel purified. The fragment was then cloned within the EcoRI/SacI sites of the pDC316 plasmid using a linker (5′-ggccagct-3′, SEQ ID NO.: 53). Clones were characterized by restriction enzyme analysis and sequencing. RESCUE AND AMPLIFICATION: pDC316 plasmids expressing murine YLK-40/BRP-39 or murine AMCase were co-transfected with Adenovirus genomic DNA into 293 cells by calcium phosphate coprecipitation method. After two rounds of plaque purification, plaques were picked, DNA isolated and characterized by restriction enzyme analysis. Plaque lysates were then amplified in 60 mm plates. Cells demonstrating CPE were harvested, subjected to freeze/thaw, and lysates used to infect cells in T-150 flasks. Cells were harvested after 2-3 days, subjected to freeze/thaw and used to infect spinner cultures. After 3-4 days of infection, cells were harvested and vectors were purified by performing cesium chloride density gradient centrifugation. Banded vectors were desalted by dialysis or column chromatography, aliquoted into vials and stored at −70° C.

Adenovector Administration: Day 1, BALB/c mice were anesthetized using Isoflurane. Recombinant adenovector (5×10⁷ or 5×10⁸ pfu, in 50 or 100 μl volumes respectively) expressing either AMCase or BRP-39 was then administered intratracheally. On day 5, mice were euthanized via CO2 asphyxiation and bled for serum via cardiac puncture. Bronchoalveolar lavage was performed and lung tissue taken for histological and biochemical analysis. RT-PCR analysis of C/CLP expression in the lung tissue was performed using standard methods.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

1-22. (canceled)

23. An isolated monoclonal antibody that specifically binds human acidic mammalian chitinase (AMCase), or antigenic fragment thereof, wherein the antibody comprises:
a) a light chain variable region and a heavy chain variable region, each having the 3 CDRs of the light and heavy chains of the antibody 171.204 produced by the hybridoma deposited as ATCC Accession Number PTA-7131, respectively; or
b) a light chain variable region having the 3 CDRs of the light chain of the antibody Z8, SEQ ID NO: 28 and a heavy chain variable region having the 3 CDRs of the heavy chain of the antibody Z8, SEQ ID NO:30;
and wherein the antibody reduces IL-13 enhanced methacholine airway hyperresponsiveness in a mammal.

24. The antibody of claim 23, wherein the antibody inhibits or reduces human AMCase-mediated inhibition of galectin-mediated apoptosis of an immune cell.

25. The antibody of claim 23, wherein the antibody reduces cellular infiltration of the lung associated with asthma.

26. The antibody of claim 23, wherein the antibody inhibits Th2-mediated inflammation.

27. The antibody of claim 23, wherein the antibody inhibits the expression or secretion of a cytokine induced by human AMCase, wherein the cytokine is selected from the group consisting of: eotaxin, eotaxin-2, monocyte chemotatic protein (MCP-1), MCP-2, macrophage inflammatory protein (MIP)-1β, C10, TNF-α, RANTES, IL-10, IL-6 and ENA-78.

28. The antibody of claim 23, wherein the antibody when administered to a mouse in the mouse OVA-induced asthma model reduces airway hyperresponsiveness (AHR) by at least 20% compared to an untreated control.

29. The antibody of claim 23, wherein the antibody when administered to a mouse in the mouse OVA-induced asthma model provides at least a 20% reduction in the accumulation of eosinophils in BAL compared to an untreated control.

30. The antibody of claim 23, wherein the antibody comprises a light chain variable region and a heavy chain variable region, each having the 3 CDRs of the light and heavy chains of the antibody 171.204 produced by the hybridoma deposited as ATCC Accession Number PTA-7131.

31. The antibody of claim 30, wherein the antibody is humanized.

32. The antibody of claim 23, wherein the antibody comprises a light chain variable region having the 3 CDRs of the light chain of the antibody Z8, SEQ ID NO: 28 and a heavy chain variable region having the 3 CDRs of the heavy chain of the antibody Z8, SEQ ID NO:30.

33. An isolated monoclonal antibody that specifically binds human AMCase, or antigenic fragment thereof, wherein the antibody comprises:
a) a heavy chain variable region from the antibody 171.204 produced by the hybridoma deposited as ATCC Accession Number PTA-7131 and a light chain variable region; or
b) a heavy chain variable region from the antibody Z8, SEQ ID NO: 30 and a light chain variable region; or
c) a light chain variable region from the antibody 171.204 produced by the hybridoma deposited as ATCC Accession Number PTA-7131 and a heavy chain variable region; or
d) a light chain variable region from the antibody Z8, SEQ ID NO:28 and a heavy chain variable region; or
e) a light chain variable region from the antibody Z8, SEQ ID NO:28 and a heavy chain variable region from the antibody Z8, SEQ ID NO: 30; or
f) a light chain variable region and a heavy chain variable region of the antibody 171.204 produced by the hybridoma deposited as ATCC Accession Number PTA-7131;
and wherein the antibody reduces IL-13 enhanced methacholine airway hyperresponsiveness in a mammal.

34. The antibody of claim 33, wherein the antibody comprises a heavy chain variable region from the antibody Z8, SEQ ID NO: 30 and a light chain variable region.

35. The antibody of claim 33, wherein the antibody comprises a light chain variable region from the antibody Z8, SEQ ID NO:28 and a heavy chain variable region.

36. The antibody of claim 33, wherein the antibody comprises a light chain variable region from the antibody Z8, SEQ ID NO:28 and a heavy chain variable region from the antibody Z8, SEQ ID NO: 30.

37. A method for treating a condition characterized by increased levels of AMCase in bronchioalveolar lavage (BAL) fluid or in synovial fluid, comprising administering a therapeutically effective amount of the antibody of claim 23.

38. A method for treating a condition characterized by increased levels of AMCase in bronchioalveolar lavage (BAL) fluid or in synovial fluid, comprising administering a therapeutically effective amount of the antibody of claim 33.

39. The method of claim 37, wherein the condition characterized by increased levels of AMCase is selected from the group consisting of: asthma, COPD, airway hypersensitivity and arthritis.

40. The method of claim 38, wherein the condition characterized by increased levels of AMCase is selected from the group consisting of: asthma, COPD, airway hypersensitivity and arthritis.

41. The method of claim 37, wherein the condition is associated with IL-13-mediated pulmonary inflammation.

42. The method of claim 38, wherein the condition is associated with IL-13-mediated pulmonary inflammation.
